(19) United States
(12) Patent Application Publication
(10) Pub. No.: US 2009/0221600 A1
(43) Pub. Date: Sep. 3, 2009

(54) PYRIDO-PYRIDIMIDINE DERIVATIVES USEFUL AS ANTIINFLAMMATORY AGENTS

(76) Inventors: Ashwani Kumar Verma, Delhi (IN); Yogesh Bhaskarrao Waman, Maharashtra (IN); Venkata P. Palle, Maharashtra (IN); Atul Kondaskar, Maharashtra (IN); Abhijit Ray, Delhi (IN); Malini Bajpai, Utter Pradesh (IN); Geeta Sharma, Delhi (IN)

Correspondence Address: RANBAXY INC., 600 COLLEGE ROAD EAST, SUITE 2100, PRINCETON, NJ, 08540, US

(21) Appl. No.: 12/088,538

(22) PCT Filed: Sep. 28, 2006
(86) PCT No.: PCT/IB06/02694

§ 371 Date: Oct. 30, 2008

(30) Foreign Application Priority Data
Sep. 28, 2005 (IN) 2602/DEL/2005

(51) Int. Cl. A61K 31/519; C07D 471/04; A61P 29/00

(52) U.S. Cl. 514/252.16; 544/279; 514/264.11

(57) ABSTRACT

Provided are novel pyrido-pyrimidine derivatives, having the structure of Formula (I): which can be used as anti-inflammatory agents. Also provided are pharmaceutical compositions comprising one or more pyrido-pyrimidine derivatives, as well as methods of treating autoimmune diseases, inflammation or associated pathologies, including for example, sepsis, rheumatoid arthritis, inflammatory bowel disease, type-1 diabetes, asthma, chronic obstructive pulmonary disorder, organ transplant rejection and psoriasis, comprising administering such compounds or pharmaceutical compositions comprising them.

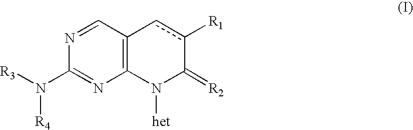

### FIELD OF THE INVENTION

Provided are novel pyrido-pyrimidine derivatives, which can be used as anti-inflammatory agents. Also provided are pharmaceutical compositions comprising one or more pyrido-pyrimidine derivatives, as well as methods of treating autoimmune diseases, inflammation or associated pathologies, including for example, sepsis, rheumatoid arthritis, inflammatory bowel disease, type-1 diabetes, asthma, chronic obstructive pulmonary disorder, organ transplant rejection and psoriasis, comprising administering such compounds or pharmaceutical compositions comprising them.

### BACKGROUND OF THE INVENTION

During the last decade, numerous studies have focused on the roles played by cytokines, a unique class of intercellular regulatory proteins, in the pathogenesis of many diseases. Cytokines play a crucial role in initiating, maintaining, and regulating immunological and inflammatory processes. Advances in our understanding of their role in immune and inflammatory disorders have led to the development of cytokine-based therapies, i.e., therapies that aim to inhibit or restore the activity of specific cytokines. Today, drugs that block inflammatory cytokines, such as tumor necrosis factor-alpha (TNF-α), are among the most successful agents being introduced to the market.

Elevated levels of proinflammatory cytokines viz TNF-α and IL-1α are associated with the pathogenesis of many immune mediated inflammatory disorders, for example, sepsis, rheumatoid arthritis, inflammatory bowel disease, type-1 diabetes, asthma, chronic obstructive pulmonary disorder, organ transplant rejection or psoriasis. Inflammation is regulated by a large number of pro- and anti-inflammatory mediators, which include cytokines, eicosanoids, nitric oxide, and reactive oxygen species. The central role of these inflammatory mediators in the pathogenesis of both chronic and acute inflammatory diseases is well documented. Until a few years ago, inflammatory disorders were treated primarily with relatively non-selective anti-inflammatory agents, such as corticosteroids and various non-steroidal anti-inflammatory drugs. In recent years, novel therapies have been developed that specifically interfere with the action of selected pro-inflammatory mediators, such as TNFα and PGE2. These specific anti-inflammatory therapies have already proven to be very successful in treating rheumatoid arthritis, inflammatory bowel disease, and several other inflammatory diseases.

The development of protein-based therapies that inhibit the activities of tumour-necrosis factor-α(TNF-α), including etanercept (Enbrel; Amgen/Wyeth), infliximab (Remicade; Centocor), and adalimumab (Humira; Abbott), has been an important advancement in treating autoimmune diseases such as rheumatoid arthritis. The approval of KINERET, an interleukin-1 (IL-1) receptor antagonist, further indicates the clinical activity of protein-based therapies that regulate cytokine activities. However, current injectable therapies have associated limitations and risks, including the potential for increased malignancies and infections and increased congestive heart failure. Studies in rodent models have provided evidence that targeting specific pathways involved in TNF-α activities are effective approaches to interrupting the pro-inflammatory process. Oral small molecules that regulate these pathways should be the next significant advancement in treating chronic inflammatory diseases when used either as a monotherapy or in combination with the current injectables.

Numerous studies have now established that the pathogenesis of inflammatory diseases require cytokine-mediated communication between endothelial cells, infiltrating leukocytes, resident macrophages, mast cells, epithelial cells and osteoclasts. The p38 mitogen activated protein kinase (p38MAPK) regulates cytokine levels and therefore plays a central role in both the cellular infiltration and activation responses associated with inflammatory diseases.

The p38 MAPK is a member of a large family of MAPK's whose signaling pathways also include the extracellular regulated kinases (ERK) & the c-jun N terminal kinases (JNK). MAP kinases are serine threonine kinases that transduce environmental stimuli to the nucleus and they themselves are activated by upstream MAPK kinases by phosphorylation on both tyrosine and threonine residues. The MAPK pathways are involved in alterations in cell physiology resulting from a variety of stimuli and control cell death, cell cycle machinery, gene transcription and protein translation. p38α MAPK was first identified as a tyrosine phosphorylated protein in LPS (Lipopolysaccharide) stimulated macrophages. The human p38α MAPK was identified as the target of pyridinyl imidazole compounds (cytokine suppressive anti-inflammatory drugs) that were known to block TNF-α and IL-1 release from LPS stimulated monocytes. After the cloning of first p38MAPK (p38α), additional members of the p38MAPK family were cloned by homology, including the p38α, p38β and p38γ.

The p38 pathway controls the activity of multiple transcription factors and the expression of many genes. There is ample evidence implicating a pivotal role for p38 in inflammatory processes mediated by IL-1 and TNF-α. p38 inhibitors have been shown to effectively block both TNFα and IL-1 biosynthesis by LPS stimulated human monocytes.

In addition, p38MAPk also plays a role in the production of IL-4, IL-6, IL-8 and IL-12. p38MAPk is also critical for cell response to certain cytokines. Treatment of human neutrophils with GM-CSF, TNF-α or TGF-α results in p38 activation. GM-CSF and TNF-α are potent enhancers of neutrophil respiratory activity suggesting a role for p38MAPk in respiratory burst.

p38 has also been implicated in the induction of cyclooxygenase-2 (COX-2) in LPS induced monocytes. COX-2 enzyme is the key enzyme in the production of prostaglandins from arachidonic acid. Inhibitors of p38MAP kinase are also expected to inhibit COX-2 expression. Accordingly inhibitors of cytokine synthesis would be expected to be effective in disorders currently treated with NSAID's. These disorders include acute and chronic pain as well as symptoms of inflammation and cardiovascular disease.

Compounds, which modulate release of one or more of the aforementioned inflammatory cytokines, can be useful in treating diseases associated with the release of these cytokines.

PCT Publication No. WO 03/057165 discloses a composition and method for preventing and treating amyloid-beta-peptide related disorders. PCT Publication No. WO 03/093290 discloses nucleoside derivatives for treating hepatitis C virus infection. PCT Publication Nos. WO 02/18380 and WO 02/18379 disclose 7-oxopyridopyrimidines as inhibitors of cellular proliferation. EP 1364950 discloses pyridopyrimidine or naphthyridine derivatives. PCT Publication No. WO 02/064594 discloses 6-substituted pyrido-pyrimidines as p-38 Kinase inhibitors. U.S. Pat. No. 5,945,422 discloses N-oxide of amino containing pyrido[2,3-d]pyrimidines. PCT Publication No. WO 98/33798 discloses pyrido[2,3-d]pyrimidines and 4-aminopyridines as inhibitors of cellular proliferation. U.S. Pat. No. 5,733,914 and PCT Publication No. WO 96/34867 disclose pyrido[2,3-d]pyrimidines for inhibiting protein tyrosine kinase mediated cellular proliferation. U.S. Patent Publication No. 2004/0019210 discloses cyclin dependent kinase and tyrosine kinase inhibitors. PCT Publication No. WO 02/03997 discloses nucleoside analogs as cell growth inhibitors. PCT Publication No. WO 03/062236 discloses 2-(pyridin-2-ylamino)-pyrido[2,3-d]pyrimidin-7-ones. PCT Publication No. WO 2004/014907 discloses-6-alkoxy-pyrido-pyrimidines as p38-MAP kinase inhibitors.

In view of the above, however, there remains a need for novel anti-inflammatory agents and in particular, novel pyridopyrimidine derivatives that can act as anti-inflammatory agents.

### SUMMARY OF THE INVENTION

Generally provided are novel pyridopyrimidine derivatives, which can be used for inhibiting or preventing autoimmune diseases, inflammation or associated pathologies, for example, sepsis, rheumatoid arthritis, inflammatory bowel disease, type-1 diabetes, asthma, chronic obstructive pulmonary disorder, organ transplant rejection or psoriasis.

Pharmaceutically acceptable salts, pharmaceutically acceptable solvates, enantiomers, diastereomers or N-oxides of these compounds having the same type of activity are also provided.

Also provided are pharmaceutical compositions comprising one or more compounds described herein and optionally one or more pharmaceutically acceptable carriers, excipients or diluents.

Compounds or pharmaceutical compositions described herein can be used for treating or preventing inflammatory, autoimmune diseases or associated pathologies, for example, sepsis, rheumatoid arthritis, inflammatory bowel disease, type-1 diabetes, asthma, chronic obstructive pulmonary disorder, organ transplant rejection or psoriasis.

### DETAILED DESCRIPTION OF THE INVENTION

In one aspect, provided are compounds having the structure of Formula I:

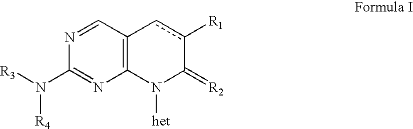

and pharmaceutically acceptable salts, pharmaceutically acceptable solvates, esters, enantiomers, diastereomers, N-oxides, polymorphs or metabolites thereof, wherein
R₁ can be alkyl, alkenyl, alkynyl, cycloalkyl, aryl, aralkyl, heteroaryl, heterocyclyl, heteroarylalkyl or heterocyclylalkyl;
R₂ can be oxygen, sulphur —NH, —N-acyl, —N(CN), —N(NO₂), —C(R₃)₂ or —CH(NO₂); [symbol]
represents a single bond or a double bond;
R₃ can be hydrogen, alkyl, cycloalkyl, aryl, heterocyclyl, heteroaryl, aralkyl, heteroarylalkyl or heterocyclylalkyl;
R₄ can be cycloalkyl or heterocyclyl; and
Het can be heterocyclyl.

In other aspects, provided are methods for treating autoimmune diseases, inflammation or associated pathologies, including for example, sepsis, rheumatoid arthritis, inflammatory bowel disease, type-1 diabetes, asthma, chronic obstructive pulmonary disorder, organ transplant rejection or psoriasis.

In another aspect, provided are pharmaceutical compositions comprising one or more compounds described herein and optionally one or more pharmaceutically acceptable carriers, excipients or diluents. Such pharmaceutical compositions can be used for treating autoimmune diseases, inflammation or associated pathologies, for example, sepsis, rheumatoid arthritis, inflammatory bowel disease, type-1 diabetes, asthma, chronic obstructive pulmonary disorder, organ transplant rejection or psoriasis.

In other aspects, provided are processes for preparing compounds disclosed herein.

The following definitions apply to terms as used herein.

The term “alkyl,” unless specified otherwise, refers to a monoradical branched or unbranched saturated hydrocarbon chain having from 1 to 20 carbon atoms. This term is exemplified by groups, such as methyl, ethyl, n-propyl, iso-propyl, n-butyl, iso-butyl, t-butyl, n-hexyl, n-decyl or tetradecyl, and the like. Alkyl groups may further be substituted with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, —COOR₆ (wherein R₆ is alkyl, cycloalkyl, aryl, aralkyl, heteroarylalkyl or heterocyclylalkyl), arylthio, thiol, alkylthio, aryloxy, alkoxyamino, —NRₓR_{y} {wherein Rₓ and R_{y} are independently selected from hydrogen, alkyl, cycloalkyl, aryl, aralkyl, —SO₂R₅ (wherein R₅ is alkyl, cycloalkyl, —NRₚR_{q} (wherein Rₚ and R_{q} are independently selected from hydrogen, alkyl, cycloalkyl, aryl, aralkyl, heterocyclyl, heteroaryl, heterocyclylalkyl or heteroarylalkyl; Rₚ and R_{q} may also together join to form a heterocyclyl ring), aryl, aralkyl, heteroaryl, heterocyclyl, heterocyclylalkyl or heteroarylalkyl), heteroaryl, heterocyclyl, heteroarylalkyl or heterocyclylalkyl}, —C(═O)NRₓR_{y}, —OC(═O)NRₓR_{y}, —NHC(═K)NRₓR_{y} (wherein K is oxygen, sulphur or nitrogen; Rₓ and R_{y} are the same as defined earlier), nitro, —S(O)ₙR₅ (wherein R₅ is the same as defined earlier and n is 0, 1 or 2). Unless otherwise constrained by the definition, all substituents may be further substituted by 1-3 substituents selected from alkyl, carboxy, —COOR₆ (wherein R₆ is the same as defined earlier), —NRₓR_{y}, —C(═O)NRₓR_{y}, —OC(═O)NRₓR_{y}, —NHC(═K)NRₓR_{y} (wherein K is the same as defined earlier), —NHC(═O)OR₆, (wherein Rₓ and R_{y} are the same as defined earlier), hydroxy, alkoxy, halogen, CF₃, cyano, and —S(O)ₙR₅ (wherein n and R₅ are the same as defined earlier). Alkyl groups may also be interrupted by 1-5 atoms of groups independently selected from oxygen, sulfur and —NRₐ (wherein Rₐ is selected from hydrogen, alkyl, cycloalkyl, alkenyl, alkynyl, aryl).

The term “alkenyl,” unless specified otherwise, refers to a monoradical of a branched or unbranched unsaturated hydrocarbon group preferably having from 2 to 20 carbon atoms with cis or trans geometry. Preferred alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom. Alkenyl groups may further be substituted with one or more substituents selected from alkyl, alkynyl, alkoxy, cycloalkyl, acyl, acylamino, acyloxy, —NRₓR_{y}, —C(═O)NRₓR_{y}, —OC(═O)NRₓR_{y}, —NHC(═K)NRₓR_{y} (wherein K, Rₓ and R_{y} are the same as defined earlier), alkoxycarbonylamino, azido, cyano, halogen, hydroxy, oxo, thiocarbonyl, carboxy, —COOR₆ (wherein R₆ is the same as defined earlier), arylthio, thiol, alkylthio, aryl, alkaryl, aryloxy, heterocyclyl, heteroaryl, heterocyclylalkyl, heteroarylalkyl, alkoxyamino, nitro, —S(O)ₙR₅ (wherein n and R₅ are the same as defined earlier). Unless otherwise constrained by the definition, all substituents may optionally be further substituted by 1-3 substituents selected from alkyl, carboxy, —COOR₆ (wherein R₆ is the same as defined earlier), hydroxy, alkoxy, halogen, —CF₃, cyano, —NRₓR_{y}, —C(═O)NRₓR_{y}, —OC(═O)NRₓR_{y} (wherein Rₓ and R_{y} are the same as defined earlier) and —S(O)ₙR₅ (wherein R₅ and n are the same as defined earlier).

The term “alkynyl,” unless specified otherwise, refers to a monoradical of an unsaturated hydrocarbon, preferably having from 2 to 20 carbon atoms. Preferred alkynyl groups include ethynyl, propargyl or propynyl, and the like. In the event that alkynyl is attached to the heteroatom, the triple bond cannot be alpha to the heteroatom. It may further be substituted with one or more substituents selected from alkyl, alkenyl, alkoxy, cycloalkyl, acyl, acylamino, alkoxyamino, acyloxy, alkoxycarbonylamino, azido, cyano, halogen, hydroxy, oxo, thiocarbonyl, carboxy, —COOR₆ (wherein R₆ is the same as defined earlier), arylthio, thiol, alkylthio, aryl, aralkyl, aryloxy, nitro, heterocyclyl, heteroaryl, heterocyclylalkyl, heteroarylalkyl, —NRₓR_{y}, —C(═O)NRₓR_{y}, —OC(═O)NRₓR_{y}, —NHC(═K)NRₓR_{y} (wherein K, Rₓ, and R_{y} are the same as defined earlier), —S(O)ₙR₅ (wherein n and R₅ are the same as defined earlier). Unless otherwise constrained by the definition, all substituents may optionally be further substituted by 1-3 substituents selected from allyl, carboxy, —COOR₆ (wherein R₆ is the same as defined earlier), hydroxy, alkoxy, halogen, CF₃, —NRₓR_{y}, —C(═O)NRₓR_{y}, —OC(═O)NRₓR_{y} (wherein Rₓ and R_{y} are the same as defined earlier), cyano and —S(O)ₙR₅ (wherein R₅ and n are the same as defined earlier).

The term “cycloalkyl,” unless specified otherwise, refers to cyclic alkyl groups of from 3 to 20 carbon atoms having a single cyclic ring or multiple condensed rings, which may optionally contain one or more olefinic bonds, unless or otherwise constrained by the definition. Cycloalkyl groups may optionally contain 1-3 heteroatoms selected from O, N and S and include, for example, oxazoline, isoxazoline, thiazoline, and the like. Such cycloalkyl groups include, by way of example, single ring structures such as cyclopropyl, cyclobutyl, cyclooctyl, cyclopentenyl, and the like, or multiple ring structures such as adamantanyl, and bicyclo [2.2.1] heptane, or cyclic alkyl groups to which is fused with an aryl group, for example indane or tetrahydro-naphthalene and the like. Cycloalkyl groups may further be substituted with one or more substituents selected from alkyl, alkenyl, alkynyl, alkoxy, cycloalkyl, acyl, acylamino, alkoxyamino, acyloxy, alkoxycarbonylamino, azido, cyano, halogen, hydroxy, oxo, thiocarbonyl, carboxy, —COOR₆ (wherein R₆ is the same as defined earlier), arylthio, thiol, alkylthio, aryl, aralkyl, aryloxy, —NRₓR_{y}, —NHC(═K)NRₓR_{y}, —C(═O)NRₓR_{y}, —OC(═O)NRₓR_{y} (wherein K, Rₓ and R_{y} are the same as defined earlier), nitro, heterocyclyl, heteroaryl, heterocyclylalkyl, heteroarylalkyl, —S(O)ₙR₅ (wherein R₅ and n are the same as defined earlier). Unless otherwise constrained by the definition, all substituents may optionally be further substituted by 1-3 substituents selected from alkyl, carboxy, hydroxy, alkoxy, halogen, —CF₃, —NRₓR_{y}, —C(═O)NRₓR_{y}, —NHC(═K)NRₓR_{y}, —OC(═O)NRₓR_{y} (wherein K, Rₓ and R_{y} are the same as defined earlier), cyano, and —S(O)ₙR₅ (wherein R₅ and n are the same as defined earlier).

The term “alkoxy,” unless specified otherwise, refers to the group O-alkyl wherein alkyl is the same as defined above.

The term “aralkyl,” unless specified otherwise, refers to aryl linked through alkyl (wherein alkyl is the same as defined above) portion and the said alkyl portion contains carbon atoms from 1-6 and aryl is as defined below.

The term “aryl,” unless specified otherwise, refers to a carbocyclic aromatic group, (for example, phenyl, biphenyl or naphthyl ring and the like optionally substituted with 1 to 3 substituents selected from halogen (P, Cl, Br, I), hydroxy, alkyl, alkenyl, acylamino, alkoxyamino, alkynyl, alkoxycarbonylamino, cycloalkyl, alkoxy, acyl, aryloxy, cyano, nitro, —NRₓR_{y}, —C(═O)NRₓR_{y}, —NHC(═K)NRₓR_{y}, —OC(═O)NRₓR_{y} (wherein K, Rₓ and R_{y} are the same as defined earlier), carboxy, —S(O)ₙR₅ (wherein R₅ and n are the same as defined earlier), —COOR₆ (wherein R₆ is the same as defined earlier), heterocyclyl, heteroaryl, heterocyclylalkyl or heteroarylalkyl.

The term “carboxy,” unless specified otherwise, herein refers to —C(═O)OH.

The term “heteroaryl,” unless specified otherwise, refers to monocyclic aromatic ring structure containing 5 or 6 carbon atoms, a bicyclic or a tricyclic aromatic group having 8 to 10 carbon atoms, with one or more heteroatom(s) independently selected from N, O and S optionally substituted with 1 to 3 substituent(s) selected from halogen (F, Cl, Br, I), hydroxy, alkyl, alkenyl, alkynyl, acylamino, alkoxyamino, alkoxycarbonylamino, cycloalkyl, acyl, carboxy, —S(O)ₙR₅ (wherein R₅ and n are the same as defined earlier), —COOR₆ (wherein R₆ is the same as defined earlier), aryl, alkoxy, aralkyl, cyano, nitro, aminocarbonylamino, —NRₓR_{y}, —C(═O)NRₓR_{y} and —OC(═O)NRₓR_{y} (wherein Rₓ and R_{y} are the same as defined earlier). Examples of heteroaryl groups are pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, xanthene, benzoxazolyl, and the like.

The term “heterocyclyl,” unless specified otherwise, refers to a non aromatic monocyclic, bicyclic (fused, bridged, or spiro) or tricyclic cycloalkyl group having 5 to 10 atoms in which 1 to 3 carbon atoms in a ring are replaced by heteroatoms selected from the group comprising of O, S and N, and are optionally benzofused or fused heteroaryl of 5-6 ring members and the said heterocyclyl group is optionally substituted wherein the substituents are selected from halogen (F, Cl, Br, I), hydroxy, alkyl, alkenyl, alkynyl, cycloalkyl, acyl, aryl, alkoxy, aralkyl, cyano, alkoxyamino, acylamino, alkoxycarbonylamino, nitro, oxo, carboxy, —S(O)ₙR₅ (wherein R₅ and n are the same as defined earlier), —COOR₆ (wherein R₆ is the same as defined earlier), —NHC(═K)NRₓR_{y}, —NHC(═K)R₆, —C(═O)NRₓR_{y}, —OC(═O)NRₓR_{y} (wherein K, Rₓ and R_{y} are the same as defined earlier). Examples of heterocyclyl groups are tetrahydrofuranyl, dihydrofuranyl, tetrahydropyranyl, dihydropyridinyl, piperidinyl, morpholine, piperazinyl, dihydrobenzofuryl, azabicyclohexyl, dihydroindolyl, and the like.

The term “heteroarylalkyl,” unless specified otherwise, refers to heteroaryl (wherein heteroaryl is same as defined earlier) linked through alkyl (wherein alkyl is the same as defined above) portion and the said alkyl portion contains carbon atoms from 1-6.

The term “heterocyclylalkyl,” unless specified otherwise, refers to heterocyclyl (wherein heterocyclyl is same as defined earlier) linked through alkyl (wherein alkyl is the same as defined above) portion and the said alkyl portion contains carbon atoms from 1-6.

The term “acyl,” unless specified otherwise, refers to —C(═O)R″ wherein R″ is selected from the group hydrogen, alkyl, cycloalkyl, aryl, aralkyl, heteroaryl, heterocyclyl, heteroarylalkyl or heterocyclylalkyl.

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “protecting groups” refers to known moieties which have the desirable property of preventing specific chemical reaction at a site on the molecule undergoing chemical modification intended to be left unaffected by the particular chemical modification. Also the term protecting group, unless or other specified may be used with groups such as hydroxy, amino, carboxy and example of such groups are found in T. W. Greene and P. G. M. Wuts, “Protective Groups in Organic Synthesis”, 2^{nd} Edn. John Wiley and Sons, New York, N.Y., which is incorporated herein by reference. The species of the carboxylic protecting groups, amino protecting groups or hydroxy protecting group employed is not critical so long as the derivatised moiety/moieties is/are stable to conditions of subsequent reactions and can be removed at the appropriate point without disrupting the remainder of the molecule.

The term “pharmaceutically acceptable salts” refers to salts of the free acids, which possess the desired pharmacological activity of the free acid and which are neither biologically nor otherwise undesirable. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). Suitable inorganic base addition salts include, but are not limited to aluminium, calcium, lithium, magnesium, potassium, sodium and zinc salts. Suitable organic base addition salts include, but are not limited to primary, secondary and tertiary amines, cyclic amines, N,N′-dibenzylethylenediamine, chloroprocaine, choline, diethanolamine, ethylenediamine and procaine salts. The pharmaceutically acceptable salts may be prepared by the conventional methods known in the prior art.

Compounds described herein may be prepared by techniques well known in the art and familiar to a practitioner skilled in art of this invention. In addition, compounds described herein may be prepared by processes described herein, such processes are not the only means by which the compounds described may be synthesized. Further, various synthetic steps described herein may be performed in any alternate sequence to form the targeted compounds.

Compounds of Formulae XI4a₁ XI5a₁ XI6a, VIII7b, VIII8b, VIII9b and XIIa may be prepared by following the reaction sequence as depicted in Scheme I. Thus a compound of Formula II (wherein hal is halogen (Cl, Br or I)) can be reacted with a compound of Formula III (wherein Q is hydrogen or het (wherein het is heterocyclyl)) to form a compound of Formula IV. The compound of Formula IV can be reduced to form a compound of Formula V. The compound of Formula V can be oxidized to form a compound of Formula VI. The compound of Formula VI can be reacted with an ester of Formula VII (wherein R′ is alkyl and R₁ is the same as defined earlier) to form a compound of Formula VIII. The compound of Formula VIII can be oxidized to form a compound of Formula IX (wherein [symbol] is a bond connected to a chiral or achiral center). The compound of Formula VIII can be reacted with a compound of Formula VIIIa (wherein the said compound of Formula VIIIa is racemic or optically active isomer and het is the same as defined earlier) to form a compound of Formula VIIIb. The compound of Formula VIIIb can be oxidized to form a compound of Formula IX. The compound of Formula IX can be reacted with an amine of Formula X (wherein the said compound of Formula X is racemic or optically active isomer, T is cycloalkyl, heterocyclyl or

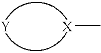

(wherein X is carbon or nitrogen; Y is carbon or Yw (wherein Yw is —NC(═O)OC(CH₃)₃ or —NC(═O)OC(CH₃)₂CHBr₂, —NC(═O)OC(CH₃)₂CCl₃, —N-aralkyl, —N-methylsulphonyl, —N-tolylsulphonyl or —N-alkyl)) to form a compound of Formula XI. The compound of Formula XI can be deprotected (wherein T is

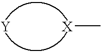

and Y is Yw) to form a compound of Formula XII. The compound of Formula XI and XII can be deprotected (wherein het is

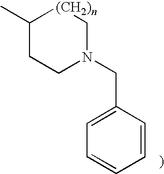

to form a compound of Formula XIa and XIIa respectively. The compound of Formula XII can be reacted with a compound of Formula Ym (wherein Ym is R₆-hal, acetic anhydride or R₆—COhal (wherein R₆ and hal are the same as defined earlier) to form a compound of Formula XIIb. The compound of Formula XIa can be reacted with a compound of Formula Rⱼⱼ-hal (wherein Rⱼⱼ is —S(O)ₙR₅ (wherein R₅ and n are the same as defined earlier), —CONRₓR_{y}, acyl and hal is the same as defined earlier) or with a compound of Formula XV (wherein K and NRₓ are the same as define earlier) to form a compound of Formula XIaa (wherein Rj is —S(O)ₙR₅, —C(═K)NHRₓ, —CONRₓR_{y} or acyl). The compound of Formula XIaa can be deprotected (wherein T is

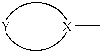

and Y is Yw) to form a compound of Formula XIaaa. The compound of Formula XIaaa can be reacted with a compound of Formula Ym (wherein Ym is the same as defined above) to form a compound of Formula XI4a (wherein K₂ is —CO or a bond and K₁ is R₆. The compound of Formula XIaaa can be reacted with a compound of Formula R₆—SO₂hal (wherein hal and R₆ are the same as defined earlier) to form a compound of Formula XI5a. The compound of Formula XIaaa can be reacted with a compound of Formula XV (wherein K and Rₓ are the same as defines earlier) to form a compound of Formula XI6a. The compound of Formula VIIIb can be deprotected (wherein het is

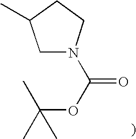

to form a compound of Formula VIII2b. The compound of Formula VIII2b can be reacted with a compound of Formula Ym (wherein Ym is the same as defined earlier) to form a compound of Formula VIII3b (wherein K₂ and K₁ are the same as defined earlier). The compound of Formula VIII3b can be oxidized to form a compound of Formula VIII4b. The compound of Formula VIII4b can be reacted with a compound of Formula X (wherein T is the same as defined earlier) to form a compound of Formula VIII5b. The compound of Formula VIII5b can be deprotected (wherein T is

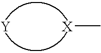

and Y is Yw) to form a compound of Formula VIII6b. The compound of Formula VIII6b can be reacted with a compound of Formula XV, R₆—SO₂hal or Ym to form a compound of Formula VIII7b, Formula VIII8b and Formula VIII9b, respectively.

The reaction of a compound of Formula II with a compound of Formula III to form a compound of Formula IV can be carried out in one or more organic solvents, for example tetrahydrofuran, dimethylformamide, dioxane or diethyl ether in the presence of one or more bases, for example, triethylamine, N-ethyldiisopropylamine, N-methylmorpholine, pyridine or mixtures thereof.

The compound of Formula IV can be reduced to form a compound of Formula V in one or more organic solvents, for example, tetrahydrofuran, dimethylformamide, dioxane, diethyl ether or mixtures thereof. The reaction can also be carried out in the presence of one or more reducing agents, for example, lithium aluminium hydride, lithium borohydride, sodium cyanoborohydride, sodium borohydride or mixtures thereof.

The oxidation of a compound of Formula V to form a compound of Formula VI can be carried out in one or more organic solvents, for example, dichloromethane, dichloroethane, carbon tetrachloride, chloroform or mixtures thereof. The reaction can also be carried out in the presence of one or more oxidizing agents, for example, manganese dioxide, potassium permanganate, Dess Martin periodinane (DMP), pyridinium dichromate (PDC), pyridinium chlorochromate (PCC), chromic anhydride or mixtures thereof. Other known oxidation methods can also be employed (see, for example, Advanced Organic Chemistry, 4^{th} Edn., Merck, John Wiley & Sons, 1992).

The reaction of a compound of Formula VI with a compound of Formula VII to form a compound of Formula VIII can be carried out in one or more organic solvents, for example, N-methylpyrrolidinone, dimethylformamide, tetrahydrofuran, diethylether, dioxane or mixtures thereof. The reaction can also be carried out in the presence of one or more bases, for example, potassium carbonate, sodium carbonate, lithium carbonate, potassium carbonate, lithium carbonate, sodium carbonate or mixtures thereof.

The oxidation of a compound of Formula VIII to form a compound of Formula IX can be carried out with one or more reagents selected from m-chloroperbenzoic acid, oxone (KHSO₅) or mixtures thereof. The reaction can also be carried out in one or more organic solvents, for example, chloroform, carbon tetrachloride, dichloromethane, ethanol, tetrahydrofuran or mixtures thereof.

The reaction of a compound of Formula VIII with a compound of Formula VIIIa to form a compound of Formula VIIIb can be carried out in one or more organic solvents, for example, tetrahydrofuran, diethylether, dioxane, toluene, benzene, dimethylformamide or mixtures thereof. The reaction can also be carried out in the presence of a redox couple. Suitable redox couple agents may be any known to a person skilled in the art. The oxidizing part of the redox couple can be selected from diisopropylazodicarboxylate (DIAD), diethylazodicarboxylate (DEAD), N,N,N′N′-tetramethylazodicarboxamide (TMAD), 1,1′-(azodicarbonyl)dipiperidine (ADDP), cyanomethylenetributylphosphorane (CMBP), 4,7-dimethyl-3,5,7-hexahydro-1,2,4,7-tetrazocin-3,8-dione (DHTD), N,N,N′N′-tetraisopropylazodicarboxamide (TIPA) or mixtures thereof. The reduction part of the redox couple can be a phosphine selected from trialkylphosphine (such as tributylphosphine), triarylphosphine (such as triphenylphosphine), tricycloalkylphosphine (such as tricyclohexylphosphine), triheteroarylphosphine or mixtures thereof. The phosphine reagent having one or more aryl, alkyl or heteroaryl substituents may also be used (such as diphenylpyridylphosphine).

The oxidation of a compound of Formula VIIIb to form a compound of Formula IX can be carried out with m-chloroperbenzoic acid, oxone (KHSO₅) or mixtures thereof. The reaction can also be carried out in one or more organic solvents, for example, chloroform, carbon tetrachloride, dichloromethane, ethanol, tetrahydrofuran or mixtures thereof.

The reaction of a compound of Formula IX with a compound of Formula X to form a compound of Formula XI can be carried in the presence of one or more bases, for example, pyridine, N-methylmorpholine, N-ethyldiisopropylamine, triethylamine or mixtures thereof. The reaction can also be carried out in one or more organic solvents, for example, ethyl acetate, dichloromethane, dichloroethane, carbon tetrachloride, chloroform or mixtures thereof.

Alternatively, rather than using a compound of Formula IX, a compound of Formula VIII can be reacted directly with a compound of Formula X to form a compound of Formula XI.

The deprotection of a compound of Formula XI (T is

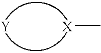

and Y is Yw (wherein Yw is —NC(═O)OC(CH₃)₃ or —NC(═O)OC(CH₃)₂CHBr₂)) to form a compound of Formula XII can be carried out in one or more alcoholic or ethereal solutions of one or more acids (for example, hydrochloric acid solution of methanol, ethanol, propanol, isopropyl alcohol or ether) or trifluoroacetic acid neat or in dichloromethane.

The deprotection of a compound of Formula XI (T is

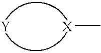

and Y is Yw (wherein Yw is —NC(═O)OC(CH₃)₂CCl₃)) to form a compound of Formula XII can be carried out by one or more supernucleophiles, such as, for example, lithium cobalt (I) phthalocyanine, zinc and acetic acid, cobalt phthalocyanine or mixtures thereof.

The deprotection of a compound of Formula XI (wherein T is

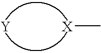

and Y is Yw (wherein Yw is —N-aralkyl)) to form a compound of Formula XII can be carried out in an organic solvent (for example, ethyl acetate, methanol, ethanol, propanol or isopropyl alcohol) in the presence of a deprotecting agent (for example, palladium on carbon in presence of hydrogen gas or palladium on carbon with a source of hydrogen gas (for example, ammonium formate, cyclohexene or formic acid)).

The deprotection of a compound of Formula XI (wherein T is

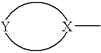

and Y is Yw (wherein Yw is —N-methylsulphonyl, —N-tolylsulphonyl)) to form a compound of Formula XII can be carried out in one or more organic solvents, for example, dimethylformamide, tetrahydrofuran or acetonitrile in the presence of one or more bases, for example, sodium hydride, potassium hydride, lithium hydride, potassium hydroxide, lithium hydroxide or sodium hydroxide (as described in T. W. Greene and P. G. M. Wuts, “Protective Groups in Organic Synthesis”, 2^{nd} Edn. John Wiley and Sons, New York, N.Y.)

The deprotection of a compound of Formula XI and a compound of Formula XII (wherein het is

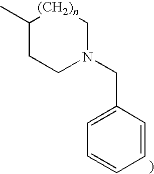

to form a compound of Formula XIa and XIIa respectively, can be carried out in an organic solvent (for example, ethyl acetate, methanol, ethanol, propanol or isopropyl alcohol) in the presence of a deprotecting agent (for example, palladium on carbon in presence of hydrogen gas or palladium on carbon with a source of hydrogen gas (for example, ammonium formate, cyclohexene or formic acid)).

The reaction of a compound of Formula XIa with a compound of Formula Rⱼⱼ-hal or with a compound of Formula XV to form a compound of Formula XIaa can be carried out in an organic solvent, for example dichloromethane, tetrahydrofuran, dimethylformamide, dioxane or diethyl ether in the presence of one or more bases, for example, triethylamine, N-ethyldiisopropylamine, N-methylmorpholine or pyridine.

The compound of Formula XIaa can be deprotected to form a compound of Formula XIaaa (wherein T is

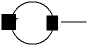

and Y is Yw (wherein Yw is —NC(═O)OC(CH₃)₃ or —NC(═O)OC(CH₃)₂CHBr₂)) in one or more alcoholic or ethereal solutions of one or more acids (for example, hydrochloric acid solution of methanol, ethanol, propanol, isopropyl alcohol, ether or mixtures thereof) or trifluoroacetic acid neat or in dichloromethane.

The compound of Formula XIaa can be deprotected to form a compound of Formula XIaaa (wherein T is

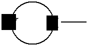

and Y is Yw (wherein Yw is —NC(═O)OC(CH₃)₂CCl₃)) by one or more supernucleophiles, such as, for example, lithium cobalt (I) phthalocyanine, zinc and acetic acid, cobalt phthalocyanine or mixtures thereof.

The compound of Formula XIaa can be deprotected to form a compound of Formula XIaaa (wherein T is

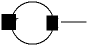

and Y is Yw (wherein Yw is -Naralkyl)) in an organic solvent (for example, ethyl acetate, methanol, ethanol, propanol or isopropyl alcohol) in the presence of a deprotecting agent (for example, palladium on carbon in presence of hydrogen gas or palladium on carbon with a source of hydrogen gas (for example, ammonium formate, cyclohexene or formic acid)).

The compound of Formula XIaa can be deprotected to form a compound of Formula XIaaa (wherein T is

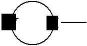

and Y is Yw (wherein Yw is —N-methylsulphonyl, —N-tolylsulphonyl)) in one or more organic solvents, for example, dimethylformamide, tetrahydrofuran or acetonitrile in the presence of one or more bases, for example, sodium hydride, potassium hydride, lithium hydride, potassium hydroxide, lithium hydroxide or sodium hydroxide (as described in T. W. Greene and P. G. M. Wuts, “Protective Groups in Organic Synthesis”, 2^{nd} Edn. John Wiley and Sons, New York, N.Y.)

The compound of Formula XIaaa can be reacted with a compound of Formula Ym (wherein Ym is R₆-hal, acetic anhydride or R₆—COhal (wherein R₆ and hal are the same as defined earlier) to form a compound of Formula XI4a₁ XI5a and XI6a, respectively in an organic solvent, for example dichloromethane, tetrahydrofuran, dimethylformamide, dioxane or diethyl ether in the presence of one or more bases, for example, triethylamine, N-ethyldiisopropylamine, N-methylmorpholine or pyridine.

The compound of Formula VIIIb can be deprotected (wherein het is

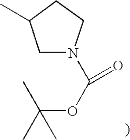

to form a compound of Formula VIII2b in one or more alcoholic or ethereal solutions of one or more acids (for example, hydrochloric acid solution of methanol, ethanol, propanol, isopropyl alcohol or ether) or trifluoroacetic acid neat or in dichloromethane.

The compound of Formula VIII2b can be reacted with a compound of Formula Ym to form a compound of Formula VIII3b in an organic solvent, for example tetrahydrofuran, dimethylformamide, dioxane or diethyl ether in the presence of one or more bases, for example, triethylamine, N-ethyldiisopropylamine, N-methylmorpholine or pyridine.

The compound of Formula VIII3b can be oxidized to form a compound of Formula VIII4b with one or more reagents selected from m-chloroperbenzoic acid or oxone (KHSO₅) in one or more organic solvents, for example, chloroform, carbon tetrachloride, dichloromethane, ethanol or tetrahydrofuran.

The compound of Formula VIII4b can be reacted with a compound of Formula X to form a compound of Formula VIII5b

The compound of Formula VIII5b can be deprotected to form a compound of Formula VIII6b (wherein T is

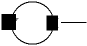

and Y is Yw (wherein Yw is —NC(═O)OC(CH₃)₃ or —NC(═O)OC(CH₃)₂CHBr₂)) in one or more alcoholic or ethereal solutions of one or more acids (for example, hydrochloric acid solution of methanol, ethanol, propanol, isopropyl alcohol or ether) or trifluoroacetic acid neat or in dichloromethane.

The compound of Formula VIII5b can be deprotected to form a compound of Formula VIII6b (wherein T is

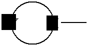

and Y is Yw (wherein Yw is —NC(═O)OC(CH₃)₂CCl₃)) in the presence of one or more supernucleophiles, such as, for example, lithium cobalt (I) phthalocyanine, zinc and acetic acid, cobalt phthalocyanine or mixtures thereof.

The compound of Formula VIII5b can be deprotected to form a compound of Formula VIII6b (wherein T is

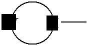

and Y is Yw (wherein Yw is —N-aralkyl)) in one or more organic solvents (for example, ethyl acetate, methanol, ethanol, propanol, isopropyl alcohol or mixtures thereof). The reaction can also be carried out in the presence of one or more deprotecting agents (for example, palladium on carbon in presence of hydrogen gas or palladium on carbon with a source of hydrogen gas (for example, ammonium formate, cyclohexene or formic acid)).

The compound of Formula VIII5b can be deprotected to form a compound of Formula VIII6b (wherein T is

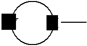

and Y is Yw (wherein Yw is —N-methylsulphonyl, —N-tolylsulphonyl)) in one or more organic solvents, for example, dimethylformamide, tetrahydrofuran, acetonitrile or mixtures thereof. The reaction can also be carried out in the presence of one or more bases, for example, sodium hydride, potassium hydride, lithium hydride, potassium hydroxide, lithium hydroxide, sodium hydroxide (as described in T. W. Greene and P. G. M. Wuts, “Protective Groups in Organic Synthesis”, 2^{nd} Edn. John Wiley and Sons, New York, N.Y.) or mixtures thereof.

The compound of Formula VIII6b can be reacted with a compound of Formula XV, R₆—SO₂hal or Ym to form a compound of Formula VIII7b, Formula VIII8b and Formula VIII9b, respectively in one or more organic solvents, for example tetrahydrofuran, dimethylformamide, dioxane, diethyl ether or mixtures thereof. The reaction can also be carried out in the presence of one or more bases, for example, triethylamine, N-ethyldiisopropylamine, N-methylmorpholine, pyridine or mixtures thereof.

Compounds prepared by the methods described herein include, for example:
tert-Butyl 4-{[6-(2-methylphenyl)-7-oxo-8-(tetrahydro-2H-pyran-4-yl)-7,8-dihydropyrido[2,3-d]pyrimidin-2-yl]amino}piperidine-1-carboxylate (Compound No. 1),
Hydrochloride salt of 6-(2-methylphenyl)-2-(piperidin-4-ylamino)-8-(tetrahydro-2H-pyran-4-yl)pyrido[2,3-d]pyrimidin-7(8H)-one (Compound No. 2),
2-[(1-Benzyl-piperidin-4-yl)amino]-6-(2-methyl-phenyl)-8-(tetrahydro-2H-pyran-4-yl)pyrido[2,3-d]pyrimidin-7(8H)-one (Compound No. 9),
6-(2-Methylphenyl)-2-[(1-methylpiperidin-4-yl)amino]-8-(tetrahydro-2H-pyran-4-yl)pyrido[2,3-d]pyrimidin-7(8H)-one (Compound No. 12),
6-(2-Methylphenyl)-2-[(4-methylpiperazin-1-yl)amino]-8-(tetrahydro-2H-pyran-4-yl)pyrido[2,3-d]pyrimidin-7(8H)-one (Compound No. 13),
tert-Butyl 4-{[6-(2-chlorophenyl)-7-oxo-8-(tetrahydro-2H-pyran-4-yl)-7,8-dihydropyrido[2,3-d]pyrimidin-2-yl]amino}piperidine-1-carboxylate (Compound No. 15),
6-(2-Methylphenyl)-2-(piperidin-1-ylamino)-8-(tetrahydro-2H-pyran-4-yl)pyrido[2,3-d]pyrimidin-7(8H)-one (Compound No. 16),
2-(Cyclobutylamino)-6-(2-methylphenyl)-8-(tetrahydro-2H-pyran-4-yl)pyrido[2,3-d]pyrimidin-7(8H)-one (Compound No. 17),
6-(2-Methylphenyl)-8-(tetrahydro-2H-pyran-4-yl)-2-(tetrahydro-2H-pyran-4-ylamino)pyrido[2,3-d]pyrimidin-7(8H)-one (Compound No. 34),
6-(2-Methylphenyl)-2-[(1,2,2,6,6-pentamethylpiperidin-4-yl)amino]-8-(tetrahydro-2H-pyran-4-yl)pyrido[2,3-d]pyrimidin-7(8H)-one (Compound No. 35),
tert-Butyl 4-{[6-(2-methylphenyl)-7-oxo-8-(tetrahydrofuran-3-yl)-7,8-dihydropyrido[2,3-d]pyrimidin-2-yl]amino}piperidine-1-carboxylate (Compound No. 36),
tert-Butyl 4-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate (Compound No. 43),
tert-Butyl 4-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 48),
Hydrochloride salt of 6-(2-methylphenyl)-2-(piperidin-4-ylamino)-8-[(3R)-tetrahydrofuran-3-yl]pyrido[2,3-d]pyrimidin-7(8H)-one (Compound No. 49),
Hydrochloride salt 6-(2-methylphenyl)-2-(piperidin-4-ylamino)-8-[(3S)-tetrahydrofuran-3-yl]pyrido[2,3-d]pyrimidin-7(8H)-one (Compound No. 50),
tert-Butyl (3S)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 70),
tert-Butyl (3S)-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate (Compound No. 72),
tert-Butyl (3R)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 73),
tert-Butyl 4-({6-(2-methylphenyl)-8-[(3S)-1-(methylsulfonyl)pyrrolidin-3-yl]-7-oxo-7,8-dihydropyrido[2,3-d]pyrimidin-2-yl}amino)piperidine-1-carboxylate (Compound No. 77),
6-(2-Methylphenyl)-2-{[1-(methylsulfonyl)piperidin-4-yl]amino}-8-[(3S)-1-(methylsulfonyl)pyrrolidin-3-yl]pyrido[2,3-d]pyrimidin-7(8H)-one (Compound No. 78),
tert-Butyl 4-({6-(2-methylphenyl)-7-oxo-8-[(3S)-pyrrolidin-3-yl]-7,8-dihydropyrido[2,3-d]pyrimidin-2-yl}amino)piperidine-1-carboxylate (Compound No. 79)
4-({8-[(3S)-1-Acetylpyrrolidin-3-yl]-6-(2-methylphenyl)-7-oxo-7,8-dihydropyrido[2,3-d]pyrimidin-2-yl}amino)-N-isopropylpiperidine-1-carboxamide (Compound No. 80),
Hydrochloride salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 81),
tert-Butyl (3S)-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxylate (Compound No. 82),
Hydrochloride salt of 6-(2-methylphenyl)-2-[(3S)-pyrrolidin-3-ylamino]-8-(tetrahydro-2H-pyran-4-yl)pyrido[2,3-d]pyrimidin-7(8H)-one (Compound No. 89),
tert-Butyl (3S)-3-{[6-(2-methylphenyl)-7-oxo-8-(tetrahydrofuran-3-yl)-7,8-dihydropyrido[2,3-d]pyrimidin-2-yl]amino}pyrrolidine-1-carboxylate (Compound No. 103),
tert-Butyl 4-({8-[(3S)-1-benzylpyrrolidin-3-yl]-6-(2-methylphenyl)-7-oxo-7,8-dihydropyrido[2,3-d]pyrimidin-2-yl}amino)piperidine-1-carboxylate (Compound No. 104),
tert-Butyl 4-{[8-(1-benzylpiperidin-4-yl)-6-(2-methylphenyl)-7-oxo-7,8-dihydropyrido[2,3-d]pyrimidin-2-yl]amino}piperidine-1-carboxylate (Compound No. 106),
tert -Butyl 4-{[6-(2-methylphenyl)-8-(1-methylpiperidin-4-yl)-7-oxo-7,8-dihydropyrido[2,3-d]pyrimidin-2-yl]amino}piperidine-1-carboxylate (Compound No. 107),
8-(1-Benzylpiperidin-4-yl)-2-(cyclobutylamino)-6-(2-methylphenyl)pyrido[2,3-d]pyrimidin-7(8H)-one (Compound No. 109),
tert-Butyl (3S)-3-{[8-(1-benzylpiperidin-4-yl)-6-(2-methylphenyl)-7-oxo-7,8-dihydropyrido[2,3-d]pyrimidin-2-yl]amino}pyrrolidine-1-carboxylate (Compound No. 110),
(3S)-3-{[8-(1-Benzylpiperidin-4-yl)-6-(2-methylphenyl)-7-oxo-7,8-dihydropyrido[2,3-d]pyrimidin-2-yl]amino}-N-isopropylpyrrolidine-1-carboxamide (Compound No. 111),
Hydrochloride salt of 8-(1-benzylpiperidin-4-yl)-6-(2-methylphenyl)-2-(piperidin-4-ylamino)pyrido[2,3-d]pyrimidin-7(8H)-one (Compound No. 112),
Hydrochloride salt of 6-(2-methylphenyl)-8-(1-methylpiperidin-4-yl)-2-(piperidin-4-ylamino)pyrido[2,3-d]pyrimidin-7(8H)-one (Compound No. 113),
tert-Butyl 4-{[6-(2-methylphenyl)-7-oxo-8-piperidin-4-yl-7,8-dihydropyrido[2,3-d]pyrimidin-2-yl]amino}piperidine-1-carboxylate (Compound No. 131),
6-(2-Methylphenyl)-8-piperidin-4-yl-2-(piperidin-4-ylamino)pyrido[2,3-d]pyrimidin-7(8H)-one (Compound No. 132),
tert-Butyl 4-{[8-{1-[(isopropylamino)carbonyl]piperidin-4-yl}-6-(2-methylphenyl)-7-oxo-7,8-dihydropyrido[2,3-d]pyrimidin-2-yl]amino}piperidine-1-carboxylate (Compound No. 134),
Hydrochloride salt of N-isopropyl-4-[6-(2-methylphenyl)-7-oxo-2-(piperidin-4-ylamino)pyrido[2,3-d]pyrimidin-8(7H)-yl]piperidine-1-carboxamide (Compound No. 135),
N-Isopropyl-4-[2-({1-[(isopropylamino)carbonyl]piperidin-4-yl}amino)-6-(2-methylphenyl)-7-oxopyrido[2,3-d]pyrimidin-8(7H)-yl]piperidine-1-carboxamide (Compound No. 136),
4-[2-({1-[(Cyclopropylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 137),
4-[2-({1-[(tert-Butylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 138),
4-[2-({1-[(Cyclohexylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 139),
N-Isopropyl-4-[6-(2-methylphenyl)-2-{[1-(morpholin-4-ylcarbonyl)piperidin-4-yl]amino}-7-oxopyrido[2,3-d]pyrimidin-8(7H)-yl]piperidine-1-carboxamide (Compound No. 140),
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 141),
N-Isopropyl-4-[6-(2-methylphenyl)-2-{[1-(methylsulfonyl)piperidin-4-yl]amino}-7-oxopyrido[2,3-d]pyrimidin-8(7H)-yl]piperidine-1-carboxamide (Compound No. 142),
4-[2-{[1-(Ethylsulfonyl)piperidin-4-yl]amino}-6-(2-methylphenyl)-7-oxopyrido[2,3-d]pyrimidin-8(7H)-yl]-N-isopropylpiperidine-1-carboxamide (Compound No. 143),
N-Isopropyl-4-[2-{[1-(isopropylsulfonyl)piperidin-4-yl]amino}-6-(2-methylphenyl)-7-oxopyrido[2,3-d]pyrimidin-8(7H)-yl]piperidine 1-carboxamide (Compound No. 144),
4-[2-{[1-(Cyclopropylcarbonyl)piperidin-4-yl]amino}-6-(2-methylphenyl)-7-oxopyrido[2,3-d]pyrimidin-8(7H)-yl]-N-isopropylpiperidine-1-carboxamide (Compound No. 145),
4-[2-[(1-Benzoylpiperidin-4-yl)amino]-6-(2-methylphenyl)-7-oxopyrido[2,3-d]pyrimidin-8(7H)-yl]-N-isopropylpiperidine-1-carboxamide (Compound No. 146),
4-[2-[(1-Acetylpiperidin-4-yl)amino]-6-(2-methylphenyl)-7-oxopyrido[2,3-d]pyrimidin-8(7H)-yl]-N-isopropylpiperidine-1-carboxamide (Compound No. 147),
N-Isopropyl-4-[6-(2-methylphenyl)-2-[(1-methylpiperidin-4-yl)amino]-7-oxopyrido[2,3-d]pyrimidin-8(7H)-yl]piperidine-1-carboxamide (Compound No. 148),
4-[2-[(1-Benzylpiperidin-4-yl)amino]-6-(2-methylphenyl)-7-oxopyrido[2,3-d]pyrimidin-8(7H)-yl]-N-isopropylpiperidine-1-carboxamide (Compound No. 149),
8-(1-Acetylpiperidin-4-yl)-2-[(1-acetylpiperidin-4-yl)amino]-6-(2-methylphenyl)pyrido[2,3-d]pyrimidin-7(8H)-one (Compound No. 150),
tert-Butyl 4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxylate (Compound No. 152),
Hydrochloride salt of 6-(2-methylphenyl)-8-[1-(methylsulfonyl)piperidin-4-yl]-2-(piperidin-4-ylamino)pyrido[2,3-d]pyrimidin-7(8H)-one (Compound No. 153),
N-Isopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 154),
N-(tert-Butyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 155),
N-Cyclohexyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 156),
N-(4-Fluorophenyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 157),
6-(2-Methylphenyl)-8-[1-(methylsulfonyl)piperidin-4-yl]-2-{[1-(morpholin-4-ylcarbonyl)piperidin-4-yl]amino}pyrido[2,3-d]pyrimidin-7(8H)-one (Compound No. 158),
6-(2-Methylphenyl)-8-[1-(methylsulfonyl)piperidin-4-yl]-2-{[1-(methylsulfonyl)piperidin-4-yl]amino}pyrido[2,3-d]pyrimidin-7(8H)-one (Compound No. 159),
2-{[1-(Ethylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 160),
2-{[1-(Isopropylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 161),
2-{[1-(Cyclopropylcarbonyl)piperidin-4-yl]amino}-6-(2-methylphenyl)-8-[1-(methylsulfonyl)piperidin-4-yl]pyrido[2,3-d]pyrimidin-7(8H)-one (Compound 162),
2-[(1-Acetylpiperidin-4-yl)amino]-6-(2-methylphenyl)-8-[1-(methylsulfonyl)piperidin-4-yl]pyrido[2,3-d]pyrimidin-7(8H)-one (Compound No. 163),
2-[(1-Benzoylpiperidin-4-yl)amino]-6-(2-methylphenyl)-8-[1-(methylsulfonyl)piperidin-4-yl]pyrido[2,3-d]pyrimidin-7(8H)-one (Compound No. 164),
2-{[1-(4-Fluorobenzoyl)piperidin-4-yl]amino}-6-(2-methylphenyl)-8-[1-(methylsulfonyl)piperidin-4-yl]pyrido[2,3-d]pyrimidin-7(8H)-one (Compound No. 165),
tert-Butyl 4-{[8-(1-acetylpiperidin-4-yl)-6-(2-methylphenyl)-7-oxo-7,8-dihydropyrido[2,3-d]pyrimidin-2-yl]amino}piperidine-1-carboxylate (Compound No. 166),
8-(1-Acetylpiperidin-4-yl)-6-(2-methylphenyl)-2-(piperidin-4-ylamino)pyrido[2,3-d]pyrimidin-7(8H)-one (Compound No. 167),
4-{[8-(1-Acetylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 168),
4-{[8-(1-Acetylpiperidin-4-yl)-6-(2-methylphenyl)-7-oxo-7,8-dihydropyrido[2,3-d]pyrimidin-2-yl]amino}-N-cyclopropylpiperidine-1-carboxamide (Compound No. 169),
4-{[8-(1-Acetylpiperidin-4-yl)-6-(2-methylphenyl)-7-oxo-7,8-dihydropyrido[2,3-d]pyrimidin-2-yl]amino}-N-(tert-butyl)piperidine-1-carboxamide (Compound No. 170),
4-{[8-(1-Acetylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 171),
4-{[8-(1-Acetylpiperidin-4-yl)-6-(2-methylphenyl)-7-oxo-7,8-dihydropyrido[2,3-d]pyrimidin-2-yl]amino}-N-(4-fluorophenyl)piperidine-1-carboxamide (Compound No. 172),
8-(1-Acetylpiperidin-4-yl)-6-(2-methylphenyl)-2-{[1-(morpholin-4-ylcarbonyl)piperidin-4-yl]amino}pyrido[2,3-d]pyrimidin-7(8H)-one (Compound No. 173),
8-(1-Acetylpiperidin-4-yl)-6-(2-methylphenyl)-2-{[1-(methylsulfonyl)piperidin-4-yl]amino}pyrido[2,3-d]pyrimidin-7(8H)-one (Compound No. 174),
8-(1-Acetylpiperidin-4-yl)-2-{[1-(ethylsulfonyl)piperidin-4-yl]amino}-6-(2-methylphenyl)pyrido[2,3-d]pyrimidin-7(8H)-one (Compound No. 175),
8-(1-Acetylpiperidin-4-yl)-2-{[1-(isopropylsulfonyl)piperidin-4-yl]amino}-6-(2-methylphenyl)pyrido[2,3-d]pyrimidin-7(8H)-one (Compound No. 176),
8-(1-Acetylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 177),
8-(1-Acetylpiperidin-4-yl)-2-{[1-(4-fluorobenzoyl)piperidin-4-yl]amino}-6-(2-methylphenyl)pyrido[2,3-d]pyrimidin-7(8H)-one (Compound No. 178),
tert-Butyl 4-{[8-[(3S)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 179),
Hydrochloride salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 180),
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 181),
2-[(1-Acetylpiperidin-4-yl)amino]-6-(2-methylphenyl)-8-(1-methylpiperidin-4-yl)pyrido[2,3-d]pyrimidin-7(8H)-one (Compound No. 182),
Hydrochloride salt of 6-(2-methylphenyl)-8-[(3S)-1-(methylsulfonyl)pyrrolidin-3-yl]-2-piperidin-4-ylamino)pyrido[2,3-d]pyrimidin-7(8H)-one (Compound No. 183),
tert-Butyl 4-{[8-[(3R)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 184),
Hydrochloride salt of 6-(2-methylphenyl)-2-[(3S)-pyrrolidin-3-ylamino]-8-(tetrahydrofuran-3-yl)pyrido[2,3-d]pyrimidin-7(8H)-one (Compound No. 185),
Hydrochloride salt of 6-(2-methylphenyl)-2-[(3S)-piperidin-3-ylamino]-8-[(3R)-tetrahydrofuran-3-yl]pyrido[2,3-d]pyrimidin-7(8H)-one (Compound No. 186),
Hydrochloride salt of 6-(2-methylphenyl)-2-[(3S)-piperidin-3-ylamino]-8-[(3S)-tetrahydrofuran-3-yl]pyrido[2,3-d]pyrimidin-7(8H)-one (Compound No. 187),
Hydrochloride salt of 6-(2-methylphenyl)-2-[(3R)-piperidin-3-ylamino]-8-[(3R)-tetrahydrofuran-3-yl]pyrido[2,3-d]pyrimidin-7(8H)-one (Compound No. 188),
4-[2-[(1-{[(4-Fluorophenyl)amino]carbonyl}piperidin-4-yl)amino]-6-(2-methylphenyl)-7-oxopyrido[2,3-d]pyrimidin-8(7H)-yl]-N-isopropylpiperidine-1-carboxamide (Compound No. 189),
4-{[8-{1-[(Isopropylamino)carbonyl]piperidin-4-yl}-6-(2-methylphenyl)-7-oxo-7,8-dihydropyrido[2,3-d]pyrimidin-2-yl]amino}-N-morpholin-4-ylpiperidine-1-carboxamide (Compound No. 190),
4-[2-{[1-(2,2-Dimethylpropanoyl)piperidin-4-yl]amino}-6-(2-methylphenyl)-7-oxopyrido[2,3-d]pyrimidin-8(7H)-yl]-N-isopropylpiperidine-1-carboxamide (Compound No. 191),
8-[(3S)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 192),
N-Cyclopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 193),
4-({6-(2-Methylphenyl)-8-[1-(methylsulfonyl)piperidin-4-yl]-7-oxo-7,8-dihydropyrido[2,3-d]pyrimidin-2-yl}amino)-N-morpholin-4-ylpiperidine-1-carboxamide (Compound No. 194),
2-{[1-(2,2-Dimethylpropanoyl)piperidin-4-yl]amino}-6-(2-methylphenyl)-8-[1-(methylsulfonyl)piperidin-4-yl]pyrido[2,3-d]pyrimidin-7(8H)-one (Compound No. 195),
6-(2-Methylphenyl)-2-[(1-methylpiperidin-4-yl)amino]-8-[1-(methylsulfonyl)piperidin-4-yl]pyrido[2,3-d]pyrimidin-7(8H)-one (Compound No. 196),
2-[(1-Benzylpiperidin-4-yl)amino]-6-(2-methylphenyl)-8-[1-(methylsulfonyl)piperidin-4-yl]pyrido[2,3-d]pyrimidin-7(8H)-one (Compound No. 197),
8-(1-Acetylpiperidin-4-yl)-2-[(1-benzoylpiperidin-4-yl)amino]-6-(2-methylphenyl)pyrido[2,3-d]pyrimidin-7(8H)-one (Compound No. 198),
8-(1-Acetylpiperidin-4-yl)-2-{[1-(2,2-dimethylpropanoyl)piperidin-4-yl]amino}-6-(2-methylphenyl)pyrido[2,3-d]pyrimidin-7(8H)-one (Compound No. 199),
8-(1-Acetylpiperidin-4-yl)-6-(2-methylphenyl)-2-[(1-methylpiperidin-4-yl)amino]pyrido[2,3-d]pyrimidin-7(8H)-one (Compound No. 200),
8-(1-Acetylpiperidin-4-yl)-2-[(1-benzylpiperidin-4-yl)amino]-6-(2-methylphenyl)pyrido[2,3-d]pyrimidin-7(8H)-one (Compound No. 201),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 202),
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 203),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 204),
4-{[8-[(3S)-1-Acetylpyrrolidin-3-yl]-6-(2-methylphenyl)-7-oxo-7,8-dihydropyrido[2,3-c]pyrimidin-2-yl]amino}-N-(4-fluorophenyl)piperidine-1-carboxamide (Compound No. 205),
4-{[8-[(3S)-1-Acetylpyrrolidin-3-yl]-6-(2-methylphenyl)-7-oxo-7,8-dihydropyrido[2,3-d]pyrimidin-2-yl]amino}1-N-morpholin-4-ylpiperidine-1-carboxamide (Compound No. 206),
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 207),
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one ((Compound No. 208),
8-[(3R)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 209),
2-[(1-Acetylpiperidin-4-yl)amino]-8-[(3R)-1-acetylpyrrolidin-3-yl]-6-(2-methylphenyl)pyrido[2,3-d]pyrimidin-7(8H)-one (Compound No. 210),
8-[(3R)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 211),
8-[(3R)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 212),
8-[(3R)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 213),
8-[(3R)-1-Acetylpyrrolidin-3-yl]-6-(2-methylphenyl)-2-[(1-methylpiperidin-4-yl)amino]pyrido[2,3-d]pyrimidin-7(8H)-one (Compound No. 214),
8-[(3R)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 215),
8-[(3S)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 216),
8-[(3S)-1-Acetylpyrrolidin-3-yl]-6-(2-methylphenyl)-2-[(1-methylpiperidin-4-yl)amino]pyrido[2,3-d]pyrimidin-7(8H)-one (Compound No. 217),
4-{[8-[(3S)-1-Acetylpyrrolidin-3-yl]-6-(2-methylphenyl)-7-oxo-7,8-dihydropyrido[2,3-d]pyrimidin-2-yl]amino}-N-isopropylpiperidine-1-carboxamide (Compound No. 218),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 219),
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 220),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 221)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(4-fluorophenyl)piperidine-1-carboxamide (Compound No. 222),
4-{[8-[(3S)-1-Acetylpyrrolidin-3-yl]-6-(2-methylphenyl)-7-oxo-7,8-dihydropyrido[2,3-d]pyrimidin-2-yl]amino}-N-morpholin-4-ylpiperidine-1-carboxamide (Compound No. 223),
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 224),
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 225),
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one (Compound No. 226),
8-[(3S)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 227),
2-[(1-Acetylpiperidin-4-yl)amino]-8-[(3S)-1-acetylpyrrolidin-3-yl]-6-(2-methylphenyl)pyrido[2,3-d]pyrimidin-7(8H)-one (Compound No. 228),
8-[(3S)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 229),
8-[(3S)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 230).

or a pharmaceutically acceptable salt, pharmaceutically acceptable solvate, ester, enantiomer, diastereomer, N-oxide, polymorph or metabolite thereof.

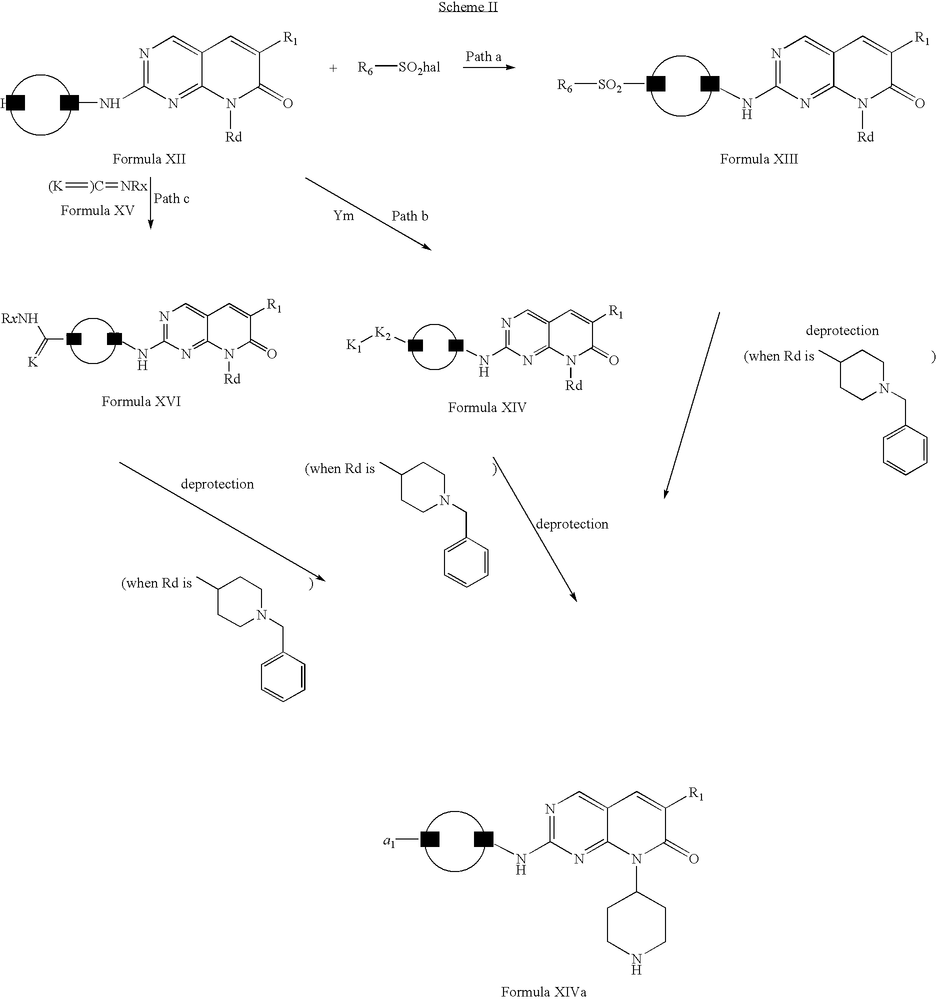

Compounds of Formulae XII, XIV or XVI may be prepared by, for example, the reaction sequence as depicted in Scheme II. In particular:

Path a: A compound of Formula XII (wherein Rd is cycloalkyl or heterocyclyl) can be reacted with R₆—SO₂-hal (wherein hal and R₆ is the same as defined earlier) to form a compound of Formula XIII. The compound of Formula XIII can be deprotected (wherein Rd is

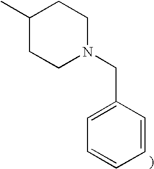

to form a compound of Formula XIVa (wherein a1 is —K₂—K₁, —SO₂R₆ or —C(═K)NHRₓ).

Path b: A compound of Formula XII can be reacted with a compound of Formula Ym (wherein Ym is R₆-hal, acetic anhydride or R₆—COhal (wherein R₆ and hal are the same as defined earlier) to form a compound of Formula XIV (wherein K₂ is —CO or a bond, and K₁ is —CH₃ or R₆). The compound of Formula XIV can be deprotected (wherein Rd is

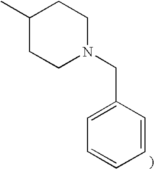

to form a compound of Formula XIVa (wherein a₁ is —K₂—K₁, —SO₂R₆ or —C(═K)NHRₓ).

Path c: A compound of Formula XII can be reacted with a compound of Formula XV (wherein K and Rₓ are the same as defined earlier) to form a compound of Formula XVI. The compound of Formula XVI can be deprotected (wherein Rd is

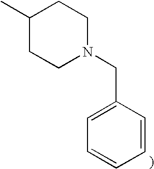

to form a compound of Formula XIVa (wherein a₁ is —K₂—K₁, —SO₂R₆ or —C(═K)NHRₓ).

The reaction of a compound of Formula XII with a compound of Formula R₆—SO₂-hal (Path a) to form a compound of Formula XIII can be carried out in the presence of one or more bases, for example, triethylamine, N-ethyldiisopropylamine, N-methylmorpholine, pyridine or mixtures thereof.

The reaction of a compound of Formula XII with a compound Y (path b) to form a compound of Formula XIV can be carried out in the presence of one or more bases, for example, triethylamine, N-ethyldiisopropylamine, N-methylmorpholine, pyridine or mixtures thereof.

The reaction of a compound of Formula XII with a compound of Formula XV (path c) to form a compound of Formula XVI can be carried out in the presence of one or more bases, for example, triethylamine, N-ethyldiisopropylamine, N-methylmorpholine, pyridine or mixtures thereof.

Compounds of Formulae XIII, XIV and XV each can be deprotected to form a compound of Formula XIVa in one or more organic solvents (for example, ethyl acetate, methanol, ethanol, propanol, isopropyl alcohol or mixtures thereof). These reactions can also be carried out in the presence of one or more deprotecting agents (for example, palladium on carbon in presence of hydrogen gas or palladium on carbon with a source of hydrogen gas (for example, ammonium formate, cyclohexene or formic acid)).

Compounds prepared by such methods include, for example:
2-[(1-Acetylpiperidin-4-yl)amino]-6-(2-methylphenyl)-8-(tetrahydro-2H-pyran-4-yl)pyrido[2,3-d]pyrimidin-7(8H)-one (Compound No. 3),
6-(2-Methylphenyl)-2-{[1-(methylsulfonyl)piperidin-4-yl]amino}-8-(tetrahydro-2H-pyran-4-yl)pyrido[2,3-d]pyrimidin-7(8H)-one (Compound No. 4),
2-[(1-Benzoylpiperidin-4-yl)amino]-6-(2-methylphenyl)-8-(tetrahydro-2H-pyran-4-yl)pyrido[2,3-d]pyrimidin-7(8H)-one (Compound No. 5),
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 6),
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 7),
2-{[1-(Ethylsulfonyl)piperidin-4-yl]amino}-6-(2-methylphenyl)-8-(tetrahydro-2H-pyran-4-yl)pyrido[2,3-d]pyrimidin-7(8H)-one (Compound No. 8),
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 10),
4-{[6-(2-Methylphenyl)-7-oxo-8-(tetrahydro-2H-pyran-4-yl)-7,8-dihydropyrido[2,3-d]pyrimidin-2-yl]amino}-N-[4-(trifluoromethyl)phenyl]piperidine-1-carboxamide (Compound No. 11),
2-{[1-(Isopropylsulfonyl)piperidin-4-yl]amino}-6-(2-methylphenyl)-8-(tetrahydro-2H-pyran-4-yl)pyrido[2,3-d]pyrimidin-7(8H)-one (Compound No. 14),
4-{[6-(2-Methylphenyl)-7-oxo-8-(tetrahydro-2H-pyran-4-yl)-7,8-dihydropyrido[2,3-d]pyrimidin-2-yl]amino}-N-propylpiperidine-1-carboxamide (Compound No. 18),
N-[(1S)-1,2-Dimethylpropyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 19),
N-Cyclohexyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 20),
2-{[1-(4-Fluorobenzoyl)piperidin-4-yl]amino}-6-(2-methylphenyl)-8-(tetrahydro-2H-pyran-4-yl)pyrido[2,3-d]pyrimidin-7(8H)-one (Compound No. 21),
N-(Cyclopentylmethyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 22),
4-{[6-(2-Methylphenyl)-7-oxo-8-(tetrahydro-2H-pyran-4-yl)-7,8-dihydropyrido[2,3-d]pyrimidin-2-yl]amino}-N-(1,1,3,3-tetramethylbutyl)piperidine 1-carboxamide (Compound No. 23),
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carboxamide (Compound No. 24),
N-Cyclopentyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 25),
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 26),
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carbothioamide (Compound No. 27),
N-tert-Butyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 28),
6-(2-Methylphenyl)-2-[(1-pyrimidin-2-ylpiperidin-4-yl)amino]-8-(tetrahydro-2H-pyran-4-yl)pyrido[2,3-d]pyrimidin-7(8H)-one (Compound No. 29),
N-Cyclopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 30),
N-[(1R)-1-Cyclohexylethyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 31),
2-{[1-(Cyclopentylcarbonyl)piperidin-4-yl]amino}-6-(2-methylphenyl)-8-(tetrahydro-2H-pyran-4-yl)pyrido[2,3-d]pyrimidin-7(8H)-one (Compound No. 32),
6-(2-Methylphenyl)-2-{[1-(pyrrolidin-1-ylcarbonyl)piperidin-4-yl]amino}-8-(tetrahydro-2H-pyran-4-yl)pyrido[2,3-d]pyrimidin-7(8H)-one (Compound No. 33),
N-Isopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 37),
6-(2-Methylphenyl)-7-oxo-2-{[1-(pyrrolidin-1-ylcarbonyl)piperidin-4-yl]amino}-1-(tetrahydrofuran-3-yl)-7,8-dihydropyrido[2,3-d]pyrimidin-1-ium (Compound No. 38),
2-{[1-(Ethylsulfonyl)piperidin-4-yl]amino}-6-(2-methylphenyl)-7-oxo-1-(tetrahydrofuran-3-yl)-7,8-dihydropyrido[2,3-d]pyrimidin-1-ium (Compound No. 39),
6-(2-Methylphenyl)-2-{[1-(methylsulfonyl)piperidin-4-yl]amino}-8-(tetrahydrofuran-3-yl)pyrido[2,3-d]pyrimidin-7(8H)-one (Compound No. 40),
N-Cyclopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 41),
6-(2-Methylphenyl)-2-{[(3S)-1-(methylsulfonyl)pyrrolidin-3-yl]amino}-8-(tetrahydrofuran-3-yl)pyrido[2,3-d]pyrimidin-7(8H)-one (Compound No. 42),
6-(2-Methylphenyl)-2-{[1-(methylsulfonyl)piperidin-4-yl]amino}-8-[(3S)-tetrahydrofuran-3-yl]pyrido[2,3-d]pyrimidin-7(8H)-one (Compound No. 44),
N-Isopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 45),
6-(2-Methylphenyl)-2-{[1-(methylsulfonyl)piperidin-4-yl]amino}-8-[(3R)-tetrahydrofuran-3-yl]pyrido[2,3-d]pyrimidin-7(8H)-one (Compound No. 46),
N-Isopropyl-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 47),
2-{[1-(Ethylsulfonyl)piperidin-4-yl]amino}-6-(2-methylphenyl)-8-[(3S)-tetrahydrofuran-3-yl]pyrido[2,3-d]pyrimidin-7(8H)-one (Compound No. 51),
N-Cyclopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 52),
N-Cyclopropyl-4-(f{6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 53),
2-[(1-Benzylpiperidin-4-yl)amino]-6-(2-methylphenyl)-8-[(3R)-tetrahydrofuran-3-yl]pyrido[2,3-d]pyrimidin-7(8H)-one (Compound No. 54),
2-(Cyclobutylamino)-6-(2-methylphenyl)-8-[(3R)-tetrahydrofuran-3-yl]pyrido[2,3-d]pyrimidin-7(8H)-one (Compound No. 55),
2-(Cyclopropylamino)-6-(2-methylphenyl)-8-[(3R)-tetrahydrofuran-3-yl]pyrido[2,3-d]pyrimidin-7(8H)-one (Compound No. 56),
2-{[1-(Ethylsulfonyl)piperidin-4-yl]amino}-6-(2-methylphenyl)-8-[(3R)-tetrahydrofuran-3-yl]pyrido[2,3-d]pyrimidin-7(8H)-one (Compound No. 57),
2-[(1-Acetylpiperidin-4-yl)amino]-6-(2-methylphenyl)-8-[(3R)-tetrahydrofuran-3-yl]pyrido[2,3-d]pyrimidin-7(8H)-one (Compound No. 58),
2-{[1-(4-Fluorobenzoyl)piperidin-4-yl]amino}-6-(2-methylphenyl)-8-[(3R)-tetrahydrofuran-3-yl]pyrido[2,3-d]pyrimidin-7(8H)-one (Compound No. 59),
2-{[1-(Cyclopropylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 60),
2-{[1-(Cyclopropylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 61),
2-{[1-(Cyclopentylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 62),
2-{[1-(Cyclopentylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 63),
2-[(1-Benzoylpiperidin-4-yl)amino]-6-(2-methylphenyl)-8-[(3S)-tetrahydrofuran-3-yl]pyrido[2,3-d]pyrimidin-7(8H)-one (Compound No. 64),
2-[(1-Benzoylpiperidin-4-yl)amino]-6-(2-methylphenyl)-8-[(3R)-tetrahydrofuran-3-yl]pyrido[2,3-d]pyrimidin-7(8H)-one (Compound No. 65),
2-{[1-(4-Fluorobenzoyl)piperidin-4-yl]amino}-6-(2-methylphenyl)-8-[(3S)-tetrahydrofuran-3-yl]pyrido[2,3-d]pyrimidin-7(8H)-one (Compound No. 66),
N-(4-Fluorophenyl)-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 67),
2-[(1-Acetylpiperidin-4-yl)amino]-6-(2-methylphenyl)-8-[(3S)-tetrahydrofuran-3-yl]pyrido[2,3-d]pyrimidin-7(8H)-one (Compound No. 68),
N-(4-Fluorophenyl)-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 69),
(3S)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 71)
(3R)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 74),
(3S)—N-Isopropyl-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 75),
6-(2-Methylphenyl)-2-{[(3S)-1-(methylsulfonyl)piperidin-3-yl]amino}-8-[(3S)-tetrahydrofuran-3-yl]pyrido[2,3-d]pyrimidin-7(8H)-one (Compound No. 76),
6-(2-Methylphenyl)-2-{[1-(methylsulfonyl)piperidin-4-yl]amino}-8-[(3S)-1-(methylsulfonyl)pyrrolidin-3-yl]pyrido[2,3-d]pyrimidin-7(8H)-one (Compound No. 77),
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 83),
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carbothioamide (Compound No. 84),
6-(2-Methylphenyl)-2-{[(3S)-1-(methylsulfonyl)pyrrolidin-3-yl]amino}-8-(tetrahydro-2H-pyran-4-yl)pyrido[2,3-d]pyrimidin-7(8H)-one (Compound No. 85),
2-{[(3S)-1-(Ethylsulfonyl)pyrrolidin-3-yl]amino}-6-(2-methylphenyl)-8-(tetrahydro-2H-pyran-4-yl)pyrido[2,3-d]pyrimidin-7(8H)-one (Compound No. 86),
2-{[(3S)-1-Acetylpyrrolidin-3-yl]amino}-6-(2-methylphenyl)-8-(tetrahydro-2H-pyran-4-yl)pyrido[2,3-d]pyrimidin-7(8H)-one (Compound No. 87),
(3S)—N-Cyclopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 88),
(3S)—N-Butyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 90),
(3S)—N-Cyclopentyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 91),
(3S)—N-[(1S)-1,2-Dimethylpropyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 92),
2-{[(3R)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 93),
2-{[(3S)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 94),
(3S)—N-Cyclohexyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 95),
(3S)-3-{[6-(2-Methylphenyl)-7-oxo-8-(tetrahydro-2H-pyran-4-yl)-7,8-dihydropyrido[2,3-d]pyrimidin-2-yl]amino}-N-octylpyrrolidine-1-carboxamide (Compound No. 96),
2-{[(3S)-1-(Cyclopropylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 97),
2-{[(3S)-1-(Cyclopentylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 98),
6-(2-Methylphenyl)-2-{[(3S)-1-pyrimidin-2-yl-pyrrolidin-3-yl]amino}-8-(tetrahydro-2H-pyran-4-yl)pyrido[2,3-d]pyrimidin-7(8H)-one (Compound No. 99)
(3S)—N-[(1R)-1-Cyclohexylethyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 100),
6-(2-Methylphenyl)-2-{[(3S)-1-(pyrrolidin-1-ylcarbonyl)pyrrolidin-3-yl]amino}-8-(tetrahydro-2H-pyran-4-yl)pyrido[2,3-d]pyrimidin-7(8H)-one (Compound No. 101),
2-{[(3S)-1-(Cyclopentylacetyl)pyrrolidin-3-yl]amino}-6-(2-methylphenyl)-8-(tetrahydro-2H-pyran-4-yl)pyrido[2,3-d]pyrimidin-7(8H)-one (Compound No. 102),
(3S)—N-Isopropyl-3-{[6-(2-methylphenyl)-7-oxo-8-(tetrahydrofuran-3-yl)-7,8-dihydropyrido[2,3-d]pyrimidin-2-yl]amino}pyrrolidine-1-carboxamide (Compound No. 105),
8-(1-Benzylpiperidin-4-yl)-6-(2-methylphenyl)-2-[(1-methylpiperidin-4-yl)amino]pyrido[2,3-d]pyrimidin-7(8H)-one (Compound No. 108),
4-{[8-(1-Benzylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 114),
4-{[8-(1-Benzylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 115),
4-{[8-(1-Benzylpiperidin-4-yl)-6-(2-methylphenyl)-7-oxo-7,8-dihydropyrido[2,3-d]pyrimidin-2-yl]amino}-N-cyclopentylpiperidine-1-carboxamide (Compound No. 116),
4-{[8-(1-Benzylpiperidin-4-yl)-6-(2-methylphenyl)-7-oxo-7,8-dihydropyrido[2,3-d]pyrimidin-2-yl]amino}-N-isobutylpiperidine-1-carboxamide (Compound No. 117),
4-{[8-(1-Benzylpiperidin-4-yl)-6-(2-methylphenyl)-7-oxo-7,8-dihydropyrido[2,3-d]pyrimidin-2-yl]amino}-N-[(1R)-1-cyclohexylethyl]piperidine-1-carboxamide (Compound No. 118),
N-Isopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 119),
N-Cyclopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 120),
8-(1-Benzylpiperidin-4-yl)-2-{[1-(cyclopentylacetyl)piperidin-4-yl]amino}-6-(2-methylphenyl)pyrido[2,3-d]pyrimidin-7(8H)-one (Compound No. 121),
8-(1-Benzylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 122),
8-(1-Benzylpiperidin-4-yl)-6-(2-methylphenyl)-2-{[1-(phenylsulfonyl)piperidin-4-yl]amino}pyrido[2,3-d]pyrimidin-7(8H)-one (Compound No. 123),
8-(1-Benzylpiperidin-4-yl)-6-(2-methylphenyl)-2-{[1-(methylsulfonyl)piperidin-4-yl]amino}pyrido[2,3-d]pyrimidin-7(8H)-one (Compound No. 124),
8-(1-Benzylpiperidin-4-yl)-2-{[1-(ethylsulfonyl)piperidin-4-yl]amino}-6-(2-methylphenyl)pyrido[2,3-d]pyrimidin-7(8H)-one (Compound no. 125),
6-(2-Methylphenyl)-8-(1-methylpiperidin-4-yl)-2-{[1-(methylsulfonyl)piperidin-4-yl]amino}pyrido[2,3-d]pyrimidin-7(8H)-one (Compound No. 126),
2-{[1-(Ethylsulfonyl)piperidin-4-yl]amino}-6-(2-methylphenyl)-8-(1-methylpiperidin-4-yl)pyrido[2,3-d]pyrimidin-7(8H)-one (Compound No. 127),
2-{[1-(Isopropylsulfonyl)piperidin-4-yl]amino}-6-(2-methylphenyl)-8-(1-methylpiperidin-4-yl)pyrido[2,3-d]pyrimidin-7(8H)-one (Compound No. 128),
6-(2-Methylphenyl)-8-(1-methylpiperidin-4-yl)-2-{[1-(pyrrolidin-1-ylcarbonyl)piperidin-4-yl]amino}pyrido[2,3-d]pyrimidin-7(8H)-one (Compound No. 129),
2-[(1-Benzoylpiperidin-4-yl)amino]-6-(2-methylphenyl)-8-(1-methylpiperidin-4-yl)pyrido[2,3-d]pyrimidin-7(8H)-one (Compound No. 130),
N-Isopropyl-4-{[6-(2-methylphenyl)-7-oxo-8-piperidin-4-yl-7,8-dihydropyrido[2,3-d]pyrimidin-2-yl]amino}piperidine-1-carboxamide (Compound No. 133),
N-Isopropyl-4-[6-(2-methylphenyl)-2-{[1-(morpholin-4-ylcarbonyl)piperidin-4-yl]amino}-7-oxopyrido[2,3-d]pyrimidin-8(7H)-yl]piperidine-1-carboxamide (Compound No. 140),
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 151).

or a pharmaceutically acceptable salt, pharmaceutically acceptable solvate, ester, enantiomer, diastereomer, N-oxide, polymorph or metabolite thereof.

Where desired, compounds described herein or their pharmaceutically acceptable salts, pharmaceutically acceptable solvates, stereoisomers, tautomers, racemates, prodrugs, metabolites, polymorphs or N-oxides may be used in combination with one or more other therapeutic agents. Examples of such therapeutic agents include one or more dopamine agonists, leukotriene antagonists, corticosteroids, PDE-IV inhibitors, caspase inhibitors, COX-2 inhibitors, MMP inhibitors, antihistamines, antitussives, TNF alpha antagonists, VLA-4 antagonists, β2-agonists or any combination thereof.

Pharmaceutical compositions for use in the methods described herein may be prepared by any of the methods of pharmacy, but all methods include the step of bringing into association the active ingredient with the carrier which constitutes one or more necessary ingredients. In general, the compositions are prepared by uniformly and intimately admixing the active ingredient with pharmaceutically acceptable liquid carriers or finely divided solid carriers or both, and then, if necessary, shaping the product into the desired presentation.

Solid form preparations include powders, tablets, pills, dispersible granules, dragees, capsules, cachets, suppositories, troches, patches, gel caps, magmas, lozenges, creams, pastes, plasters, lotions, discs, or ointments. Liquid form preparations include solutions, suspensions, emulsions, microemulsions, syrups, elixirs, aerosols, nasal spays or oral sprays.

Solid carriers can include one or more substances, which may also act as diluents, flavoring agents, solubilizers, lubricants, suspending agents, binders, or disintegrating agents. Solid carriers can also include finely divided solids, which can be in admixture with one or more finely divided compounds described herein.

In preparing tablets, one or more compounds described herein can be mixed with one or more carriers having the necessary binding properties in suitable proportions and compacted into the desired shape and size. In some embodiments, powders and tablets can contain from about 5 to about 70 percent of one or more compounds described herein. Suitable solid carriers include, for example, sucrose, glucose, lactose, pectin, mannitol, silicic acid, dextrin, starch, gelatin, tragacanth, low melting wax, cocoa butter sugars, sodium citrate, dicalcium phosphate, microcrystalline cellulose, granulating agents, lubricants, binders, disintegrating agents, absorption accelerators, wetting agents, adsorbents and the like. Binders include, for example, carboxymethylcellulose, alginates, gelatins, polyvinylpyrrolidinone, sucrose, acacia; disintegrating agents include, for example, agar-agar, calcium carbonate, potato starch, alginic acid, certain silicates and sodium carbonate; absorption accelerators include, for example, quaternary ammonium compounds; wetting agents include, for example, cetyl alcohol, glycerol mono stearate; adsorbents include, for example, Kaolin; lubricants include, for example, talc, calcium stearate, magnesium stearate, solid polyethyleneglycol, sodium lauryl sulphate and mixture thereof. In the case of capsules, tablets, pills, the dosage form may also comprise buffering agents. For example, a tablet may be prepared by compression or molding, optionally, with one or more accessory ingredients. Compressed tablets may be prepared by compressing in a suitable machine the active ingredient in a free-flowing form such as powder or granules, optionally mixed with one or more binders, lubricants, inert diluents, surface active or dispersing agents. Molded tablets may be made by molding, in a suitable machine, a mixture of a powdered form of one or more compounds moistened with one or more inert liquid diluents.

For liquid form preparations, active compounds can be mixed with water or other solvent, solubilizing agents and emulsifiers, for example, ethyl alcohol, isopropanol, ethyl carbonate, ethyl acetate, benzyl alcohol, benzyl benzoate, propylene glycol, 1,3-butylene glycol, dimethylformamide, oils (for example, cottonseed, groundnut, corn, germ, olive, castor and sesame oil), glycerol, fatty acid esters of sorbitan or mixtures thereof.

Aqueous solutions suitable for oral use can be prepared by dissolving the active component in water and adding suitable colorants, flavors, stabilizers, wetting agents, emulsifying agents, suspending agents, sweetening agents, flavoring agents, perfuming agents and thickening agents as desired. Aqueous suspension suitable for oral use can be made by dispersing the finely divided active component in water with viscous material, for example, natural or synthetic gums, resins, methyl cellulose, sodium carboxymethyl cellulose and other suspending agents. Other liquid form preparations include, for example, water or water-propylene glycol solutions for parenteral injection. Other injectable preparations, for example, sterile injections, injectable depot forms, aqueous suspensions may be formulated according to the art using suitable dispersing or wetting and suspending agent. Among the acceptable vehicles and solvents that may be employed are water, Ringer's solution and isotonic sodium chloride.

Such solutions are prepared so as to be acceptable to biological systems with respect to isotonicity, pH, and other parameters. Liquid preparations can also be formulated in solution in aqueous polyethylene glycol solution.

Ointment preparations can contain one or more compounds described herein or salts thereof with a physiologically acceptable carrier. Such salts can be heavy metal salts. The carrier can desirably be a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

Dosage forms for tropical or transdermal administration of one or more compounds described herein includes ointments, pastes, creams, lotions, gels, powders, solutions, sprays, inhalants or patches. Active compounds can be admixed under sterile condition with one or more pharmaceutically acceptable carriers and any desired preservatives or buffers as may be required. Ophthalmic formulations, eardrops, eye ointments, powders and solutions are also encompassed within the scope of this invention.

The pharmaceutical preparation can be in unit dosage form. In such forms, the preparation can be subdivided into unit doses containing appropriate quantities of the active component, i.e., one or more compounds described herein and optionally one or more other therapeutic agents. Dosage forms can be a packaged preparation containing one or more discrete unit dosages, for example, capsules; tablets; powders in vials, capsules or ampoules; ointments; cachets; gels or gel caps; cream itself; dispersible granules; suppositories; troches; patches; magmas; lozenges; pastes; plasters; lotions; discs; ointments; solutions; suspensions, emulsions, syrups, elixirs, aerosols, nasal spays or oral sprays.

The magnitude of a prophylactic or therapeutic dose of one or more compounds described herein in the acute or chronic prevention, treatment, or management of a disorder or condition will vary with the severity of the condition to be treated and the route of administration. The dose, and perhaps the dose frequency, will also vary according to the age, body weight, and response of the individual patient. Suitable total daily dose ranges can be readily determined by those skilled in the art. In general, the total daily dose range for one or more compounds described herein, for the conditions described herein, is from about 1 mg to about several grams administered in single or divided doses according to the particular application and the potency of the active ingredient. Suitable dosage amounts can be determined using small dosages that are less than the optimum dose. Such small dosages can be increased in small increments until the optimum effect is reached. Dosage amounts may be divided and administered as divided doses if desired.

Pharmaceutical compositions of the present invention may be administered by following routes for example, intravenous, intratracheal, intraarterial, intraperitoneal, intrathecal, intraventricular, intraurethral, intrasternal, intracranial, intramuscular, subcutaneous, intranasally, inhalation, rectally or vaginally.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example A

### Synthesis of tetrahydro-4H-pyran-4-amine Hydrochloride

Step a: Tetrahydro-4H-pyran-4-one Oxime

To a solution of tetrahydro-4H-pyran-4-one (commercially available) (20 g, 200 mmol) in ethanol (300 mL) was added hydroxylamine hydrochloride (41.7 g, 600 mmol). An aqueous solution of sodium acetate (57.4 g, 700 mmol) was added into the reaction mixture at 40-50° C. and refluxed overnight. The reaction mixture was concentrated under reduced pressure and the residue thus obtained was then extracted with ethyl acetate. The combined organic layer was then washed with water, dried over anhydrous sodium sulphate, filtered and evaporated under reduced pressure to yield the title compound. Yield=17 g.

Step b: Tetrahydro-4H-pyran-4-amine Hydrochloride

To a solution of the compound obtained from step a above was added dropwise into a suspension of lithium aluminum hydride (6.6 g, 173 mmol) in tetrahydrofuran (200 mL) at −20° C. The reaction mixture was allowed to warm to room temperature and refluxed for 2-4 hours. Excess lithium aluminum hydride was decomposed using ethyl acetate, aqueous sodium hydroxide and finally with water. The organic layer was concentrated under reduced pressure and the residue thus obtained was purified by addition of etheral hydrochloride at 0° C. to yield the title compound. Yield=2 g.

Scheme I, Route I:

### Example 1

### Synthesis of tert-butyl 4-{[6-(2-methylphenyl)-7-oxo-8-(tetrahydro-2H-pyran-4-yl)-7,8-dihydropyrido-[2,3-d]pyrimidin-2-yl]amino}piperidine-1-carboxylate (Compound No. 1)

Step a: 2-Methylsulfanyl-4-(tetrahydro-2H-pyran-4-ylamino)pyrimidine-5-carboxylic Acid Ethyl Ester

To a suspension of ethyl-4-chloro-2-methylthio-5-pyrimidine carboxylate (commercially available) (3.0 g, 12 mmol) in dry tetrahydrofuran (50 mL) was added triethylamine (2.6 g, 25 mmol) and tetrahydro-2H-pyran-4-ylamine hydrochloride (1.94 g, 14 mmol) at room temperature and the mixture was stirred for 4 hours. The organic solvent was evaporated under reduced pressure followed by addition of cold water. The residue thus obtained was filtered, washed with water and dried under vacuum to yield the title compound. Yield=2.7 g.

Step b: 2-Methylsulfanyl-4-(tetrahydro-2H-pyran-4-ylamino)pyrimidine-5-yl]methanol

To a solution of the compound obtained from step a above (2.7 g, 9.09 mmol) in tetrahydrofuran (20 mL) was added dropwise a suspension of lithium aluminum hydride (0.69 g, 18.1 mmol) in dry tetrahydrofuran (50 mL) at −70° C. The reaction mixture was stirred between −70° C. and −60° C. for 1 hour followed by stirring at room temperature until completion. The reaction mixture was cooled to 0° C. and diluted with ethyl acetate, followed by dropwise addition of 30% aqueous solution of sodium hydroxide. The reaction mixture was then filtered through a celite pad and washed with ethyl acetate and dichloromethane. The filtrate was evaporated under reduced pressure followed by addition of water. The residue thus obtained was filtered and dried under vacuum to yield the title compound. Yield=2.4 g.

Step c: 2-Methylsulfanyl-4-(tetrahydro-2H-pyran-4-ylamino)pyrimidine-5-carbaldehyde

To a suspension of compound obtained from step b above (2.4 g, 9.4 mmol) in dichloromethane (70 mL) was added manganese dioxide (4.85 g, 56 mmol) at room temperature and the mixture was stirred for 24-36 hours. The reaction mixture was filtered over a celite pad and evaporated under reduced pressure. The residue thus obtained was purified by column chromatography using ethyl acetate in hexane (1:4) solvent mixture as eluent to yield the title compound. Yield=2 g.

Step d: 2-Methylsulfanyl-8-(tetrahydro-2H-pyran-4-yl)-6-(2-methylphenyl)-8H-pyrido[2,3-d]pyrimidine-7-one

To a solution of the compound obtained from step c above (2 g, 7.9 mmol) in N-methylpyrrolidinone (40 mL) was added methyl (2-methyl phenyl)acetate (1.55 g, 9.48 mmol) and cesium carbonate (7.72 g, 23.7 mmol) and the mixture was heated at 110° C. for 2 hours. The reaction mixture was diluted with ethyl acetate and poured into water. The mixture was then extracted with ethyl acetate, washed with water, dried over anhydrous sodium sulphate, filtered and evaporated under reduced pressure. The residue thus obtained was purified by column chromatography using ethyl acetate in hexane (1:3) as eluent. Yield=1.8 g.

Step e: 2-Methylsulfonyl-8-(tetrahydro-2H-pyran-4-yl)-6-(2-methylphenyl)-8H-pyrido[2,3-d]pyrimidine-7-one

To a solution of the compound obtained from step d above (1.8 g, 4.9 mmol) in chloroform (20 mL) was added m-chloroperbenzoic acid (77%) (3.29 g, 14.7 mmol) at 0° C. and the mixture was stirred for 1 hour. A saturated solution of aqueous sodium bisulphite was added to the reaction followed by addition of aqueous sodium bicarbonate solution at 0° C. The reaction mixture was then extracted with dichloromethane and the organic layer was washed with water, dried over anhydrous sodium sulphate, filtered and evaporated under reduced pressure. The residue thus obtained was washed thoroughly with hexane to yield the title compound. Yield=1.5 g.

Step f:_tert-Butyl 4-{[6-(2-methyl-phenyl)-7-oxo-8-(tetrahydro-2H-pyran-4-yl)-7,8-dihydropyrido[2,3-d]pyrimidin-2-yl]amino}piperidine-1-carboxylate

To the solution of the compound obtained from step e above (0.25 g, 0.626 mmol) in pyridine was added 4-amino-piperidine-1-carboxylic acid tert-butyl ester (commercially available) (0.25 g, 1.2 mmol) and the mixture was heated to 80° C. for 1 hour. The reaction mixture was diluted with dichloromethane and the compound was purified by column chromatography using (1:1) ethyl acetate in hexane as eluent to yield the title compound. Yield=180 mg.

¹H NMR (400 MHz, CDCl₃) δ: 8.42 (s, 1H, Ar—H), 7.40 (s, 1H, Ar—H), 7.30-7.17 (m, 4H, Ar—H), 5.65 (m, 1H, N—CH), 5.57 (m, 1H, N—H exchangeable), 4.13-4.10 (m, 5H, —OCH₂, N—CH₂, N—CH), 3.56-3.50 (m, 2 Hz, —CH₂), 3.18-3.16 (m, 2H), 3.00 (m, 2H, N—CH₂), 2.22 (s, 3H, Ar—CH₃), 2.11-2.08 (m, 2H), 1.60 (m, 2H), 1.48 (bs, 11H, t-butyl+-CH₂).

Mass (+ve ion mode m/z): 520 (M⁺+1), 464 (M⁺-t-butyl), 420 (M⁺-Boc).

m.p: 197.8-199.1° C.

Analogs of tert-butyl 4-{[6-(2-methylphenyl)-7-oxo-8-(tetrahydro-2H-pyran-4-yl)-7,8-dihydropyrido[2,3-d]pyrimidin-2-yl]amino}piperidine-1-carboxylate (Compound No. 1) described below can be prepared by using the appropriate corresponding amine in place of 4-amino-piperidine-1-carboxylic acid tert-butyl ester, respectively, as applicable in each case.
2-[(1-Benzyl-piperidin-4-yl)amino]-6-(2-methyl-phenyl)-8-(tetrahydro-2H-pyran-4-yl)pyrido[2,3-d]pyrimidin-7(8H)-one (Compound No. 9)

Mass: (+ve ion mode m/z): 510 (M⁺+1)

m.p: 201.8-203.2° C.
6-(2-Methylphenyl)-2-[(1-methylpiperidin-4-yl)amino]-8-(tetrahydro-2H-pyran-4-yl)pyrido[2,3-d]pyrimidin-7(8H)-one (Compound No. 12)
6-(2-Methylphenyl)-2-[(4-methylpiperazin-1-yl)amino]-8-(tetrahydro-2H-pyran-4-yl)pyrido[2,3-d]pyrimidin-7(8H)-one (Compound No. 13)
tert-Butyl 4-{[6-(2-chlorophenyl)-7-oxo-8-(tetrahydro-2H-pyran-4-yl)-7,8-dihydropyrido[2,3-d]pyrimidin-2-yl]amino}piperidine-1-carboxylate (Compound No. 15)
6-(2-Methylphenyl)-2-(piperidin-1-ylamino)-8-(tetrahydro-2H-pyran-4-yl)pyrido[2,3-d]pyrimidin-7(8H)-one (Compound No. 16)
2-(Cyclobutylamino)-6-(2-methylphenyl)-8-(tetrahydro-2H-pyran-4-yl)pyrido[2,3-d]pyrimidin-7(8H)-one (Compound No. 17)
6-(2-Methylphenyl)-8-(tetrahydro-2H-pyran-4-yl)-2-(tetrahydro-2H-pyran-4-ylamino)pyrido[2,3-d]pyrimidin-7(8H)-one (Compound No. 34)
6-(2-Methylphenyl)-2-[(1,2,2,6,6-pentamethylpiperidin-4-yl)amino]-8-(tetrahydro-2H-pyran-4-yl)pyrido[2,3-d]pyrimidin-7(8H)-one (Compound No. 35)
tert-Butyl (3S)-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxylate (Compound No. 82)
tert-Butyl 4-({8-[(3S)-1-benzylpyrrolidin-3-yl]-6-(2-methylphenyl)-7-oxo-7,8-dihydropyrido[2,3-d]pyrimidin-2-yl}amino)piperidine-1-carboxylate (Compound No. 104),
tert-Butyl 4-{[8-(1-benzylpiperidin-4-yl)-6-(2-methylphenyl)-7-oxo-7,8-dihydropyrido[2,3-d]pyrimidin-2-yl]amino}piperidine-1-carboxylate (Compound No. 106)
tert -Butyl 4-{[6-(2-methylphenyl)-8-(1-methylpiperidin-4-yl)-7-oxo-7,8-dihydropyrido[2,3-d]pyrimidin-2-yl]amino}piperidine-1-carboxylate (Compound No. 107)
8-(1-Benzylpiperidin-4-yl)-2-(cyclobutylamino)-6-(2-methylphenyl)pyrido[2,3-d]pyrimidin-7(8H)-one (Compound No. 109)
(3S)-3-{[8-(1-Benzylpiperidin-4-yl)-6-(2-methylphenyl)-7-oxo-7,8-dihydropyrido[2,3-d]pyrimidin-2-yl]amino}-N-isopropylpyrrolidine-1-carboxamide (Compound No. 111)

### Example 2

### Synthesis of Hydrochloride Salt of 6-(2-methylphenyl)-2-(piperidin-4-ylamino)-8-(tetrahydro-2H-pyran-4-yl)pyrido[2,3-d]pyrimidin-7(8H)-one (Compound No. 2)

To a solution of the Compound No. 1 (0.15 g, 34 mmol) in dichloromethane (2 mL) was added ethereal hydrochloric acid (10 mL) at 0° C. and the mixture was stirred for 4 hours. The organic solvent was evaporated under reduced pressure followed by the addition of diethyl ether. A white solid thus obtained was filtered, washed with ether and dried under vacuum to yield the title compound. Yield=0.13 g.

¹H NMR (400 MHz, D₂O) δ: 8.54 (s, 1H, Ar—H), 7.64 (s, 1H, Ar—H), 7.33-7.23 (m, 3H, Ar—H), 7.16-7.14 (m, 1H, Ar—H), 5.6 (m, 1H, N—CH), 4.2 (m, 1H, N—CH), 4.09-4.07 (m, 2H, —OCH₂), 3.58-3.48 (m, 4H, N—CH₂+—OCH₂), 3.18-3.12 (m, 2H, N—CH₂), 2.96 (m, 2H), 2.23-2.29 (m, 2H), 2.08 (s, 3H, Ar—CH₃), 1.85-1.82 (m, 2H), 1.69-1.66 (m, 2H).

Mass (+ve ion mode m/z): 420 (M⁺+1)

m.p: 249.8-251.9° C.

The following analogs can be prepared similarly using the appropriate corresponding starting materials:
Hydrochloride salt of 6-(2-methylphenyl)-2-[(3S)-pyrrolidin-3-ylamino]-8-(tetrahydro-2H-pyran-4-yl)pyrido[2,3-d]pyrimidin-7(8H)-one (Compound No. 89)
Hydrochloride salt of 8-(1-benzylpiperidin-4-yl)-6-(2-methylphenyl)-2-(piperidin-4-ylamino)pyrido[2,3-d]pyrimidin-7(8H)-one (Compound No. 112)
Hydrochloride salt of 6-(2-methylphenyl)-8-(1-methylpiperidin-4-yl)-2-(piperidin-4-ylamino)pyrido[2,3-d]pyrimidin-7(8H)-one (Compound No. 113)

### Example 2a

### Synthesis of tert-butyl 4-{[6-(2-methylphenyl)-7-oxo-8-piperidin-4-yl-7,8-dihydropyrido[2,3-]pyrimidin-2-yl]-amino}piperidine-1-carboxylate Compound No. 131)

Step a: Ethyl 4-(4-(N-benzylpiperidine))amino-2-(methylthio)pyrimidine-5-carboxylate

To a suspension of ethyl-4-chloro-2-methylthio-5-pyrimidine-carboxylate (commercially available) (20.0 g, 85.9 mmol) in dry tetrahydrofuran (150 mL) was added triethylamine (13.01 g, 128.9 mmol) and 4-amino-N-benzylpiperidine (17.5 g, 258.2 mmol) at 0° C. and the mixture was stirred for 3 hours at room temperature. The organic solvent was evaporated under reduced pressure. The residue was dissolved in ethyl acetate and washed with water, brine, dried with Na₂SO₄, and evaporated to yield product. Yield 21.1 g.

Step b: [4-(N-Benzyl-4-piperidine)amino-2-(methylthio)pyrimidin-5-yl]methanol

To a suspension of lithium aluminium hydride (3.10 g, 81.6 mmol) in dry tetrahydrofuran (100 mL) at −70° C. was added dropwise a solution of the compound obtained from step a above (21.1 g, 68.0 mmol) in tetrahydrofuran (70 mL). The reaction mixture was stirred between −70° C.−60° C. for 1 hour and then at room temperature until completion. The reaction mixture was cooled to 0° C. and diluted with ethyl acetate, followed by addition of 30% aqueous solution of sodium sulphate. The reaction mixture was then filtered through a celite pad and washed with ethyl acetate. The filtrate was extracted with ethyl acetate and the organic layer was washed with water, dried over anhydrous sodium sulphate, filtered and evaporated under reduced pressure. Solid compound was obtained. Yield=17.9 g.

Step c: 4-(N-Benzyl-4-piperidine)amino-2-(methylthio)pyrimidine-5-carbaldehyde

To a suspension of compound obtained from step b above (17.9 g, 52.0 mmol) in dichloromethane (300 mL) was added manganese dioxide (31.7 g, 364.2 mmol) at room temperature and the mixture was stirred for 24-36 hours. The reaction mixture was filtered over a celite pad and evaporated under reduced pressure to yield the title compound as a solid.

Yield=12.4 g.

Step d: 4-(N-Benzyl-4-piperidine)-6-(2-Methylphenyl)-2-(methylthio)pyrido[2,3-d]pyrimidin-7(8H)-one

To a solution of the compound obtained from step c above (12.4 g, 36.25 mmol) in N-methylpyrrolidinone (70 mL) was added 2-methyl phenyl acetic acid methyl ester (7.1 g, 43.5 mmol) and cesium carbonate (35.4 g, 108.7 mmol) and the mixture was heated at 110° C. for 3 hours. The reaction mixture was diluted with ethyl acetate and poured into water. It was then extracted with ethyl acetate, washed with water, dried over anhydrous sodium sulphate, filtered and evaporated under reduced pressure. The residue thus obtained was purified by column chromatography using ethyl acetate in hexane (1:1) as eluent.

Yield=8.2 g.

Step e: 8-(1-Benzylpiperidin-4-yl)-6-(2-methylphenyl)-2-(methylsulfonyl)pyrido[2,3-d]pyrimidin-7(8H)-one

To a solution of the compound obtained from step d above (8.2 g, 18.0 mmol) in chloroform (100 mL) was added m-chloroperbenzoic acid (77%) (12.1 g, 54.0 mmol) at 0° C. and the mixture was stirred at room temperature for 30 minutes. A saturated solution of aqueous sodium bisulphite was added to the mixture followed by aqueous sodium bicarbonate solution at 0° C. The reaction mixture was then extracted with dichloromethane and the organic layer was washed with water, dried over anhydrous sodium sulphate, filtered and evaporated under reduced pressure. The residue thus obtained was purified by column chromatography. Yield=6.0 g

Step f: tert-Butyl 4-{[8-(1-benzylpiperidin-4-yl)-6-(2-methylphenyl)-7-oxo-7,8-dihydropyrido[2,3-d]pyrimidin-2-yl]amino}piperidine-1-carboxylate

To the compound obtained from step e above (6.0 g, 12.3 mmol) was added tert-butyl 4-aminopiperidine-1-carboxylate (2.70 g, 13.5 mmol) and the mixture was heated to 65° C. for 2 hours. The reaction mixture was diluted with ethyl acetate and the compound was purified by column chromatography using ethyl acetate in hexane (9:1) solvent mixture as eluent to yield the title compound. Yield=3.4 g.

Step g: tert-Butyl 4-{[6-(2-methylphenyl)-7-oxo-8-piperidin-4-yl-7,8-dihydropyrido[2,3-d]pyrimidin-2-yl]amino}piperidine-1-carboxylate

To the compound obtained from step f above (3.4 g, 5.6 mmol) in methanol was added 10% Pd/C (0.34 g, 10% w/w) and treated with hydrogen gas (50 psi pressure) for 30 min in a parr pressure apparatus. The reaction mixture was filtered through a celite bed and the filtrate was evaporated to yield the title compound.

Yield: 2.6 g

¹H-NMR (400 MHz, CDCl₃) δ: 8.52 (s, 1H), 7.71-7.16- (m, 4H), 5.43-5.35 (m, 1H), 5.43-5.35 (m, 1H), 4.09-4.06 (m, 4H), 3.36-3.31 (m, 1H), 3.06-2.97 (m, 6H), 2.21 (s, 3H), 2.12-2.01 (m, 2H), 1.68-1.60 (m, 2H), 1.46 (s, 13H)

Mass (+ve ion mode m/z): 519 (M⁺+1)

The following analogs can be prepared similarly using the appropriate corresponding starting materials:
tert-Butyl 4-({6-(2-methylphenyl)-7-oxo-8-[(3S)-pyrrolidin-3-yl]-7,8-dihydropyrido[2,3-d]pyrimidin-2-yl}amino)piperidine-1-carboxylate (Compound No. 79)

### Example 2b

### Synthesis of 6-(2-methylphenyl-8-piperidin-4-yl-2-(piperidin-4-ylamino)pyrido[2,3-d]pyrimidin-7(8H)-one (Compound No. 132)

The title compound was prepared following the procedure as described in Example 2 by using the Compound No. 131 in place of Compound No. 1 and using ethanolic HCl in place of etheral HCl. The crude product thus obtained was treated with aqueous sodium bicarbonate solution and extracted with ethyl acetate. The organic layer was collected, dried over anhydrous sodium sulphate and concentrated under reduced pressure to yield the title compound.

¹H-NMR (CDCl₃): δ 8.42 (s, 1H), 7.52-7.17 (m, 4H), 5.54-5.43 (m, 3H), 4.03-3.90 (m, 2H), 3.33-3.18 (m, 4H), 2.93-2.76 (m, 4H), 2.21-2.13 (m, 7H), 1.69-1.52 (m, 4H).

Mass (+ve ion mode m/z): 419 (M⁺+1)

The following analog can be prepared similarly using the appropriate corresponding starting materials:
8-(1-Acetylpiperidin-4-yl)-6-(2-methylphenyl)-2-(piperidin-4-ylamino)pyrido[2,3-d]pyrimidin-7(8H)-one (Compound No. 167)

### Example 2c

### Synthesis of tert-butyl 4-{[8-{1-[(isopropylamino)carbonyl]piperidin-4-yl}-6-(2-methylphenyl)-7-oxo-7,8-dihydropyrido[2,3-d]pyrimidin-2-yl]amino}piperidine-1-carboxylate (Compound No. 134)

To a solution of the Compound No. 131 (2.0 g, 3.86 mmol) in dichloromethane was added triethylamine (0.34 g, 11.6 mmol) and isopropyl isocyanate (0.36 g, 4.25 mmol) and the mixture was stirred at room temperature for 2 hours. The reaction mixture was diluted with dichloromethane and washed with water, brine and dried with sodium sulphate and evaporated to yield the title compound. Yield: 2.1 g.

¹H NMR (CDCl₃+CD₃OD): δ 8.40 (s, 1H), 7.41-7.14 (m, 4H), 5.52-5.45 (m, 1H), 4.23-3.93 (m, 6H), 3.11-3.05 (m, 2H), 2.98-2.85 (m, 4H), 2.19 (s, 3H), 2.03-1.94 (m, 2H), 1.73-1.62 (m, 4H), 1.46 (s, 9H), 1.19-1.14 (m, 6H).

Mass (+ve ion mode m/z): 604 (M⁺+1)

The analog compounds described below can be prepared by using the appropriate corresponding reactants, for example, acyl halide, alkyl halide, sulphonyl halide, isocyanate or isothiocyanate in place of acetic anhydride, respectively, as applicable in each case:
tert-Butyl 4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxylate (Compound No. 152)
tert-Butyl 4-{[8-(1-acetylpiperidin-4-yl)-6-(2-methylphenyl)-7-oxo-7,8-dihydropyrido[2,3-d]pyrimidin-2-yl]amino}piperidine-1-carboxylate (Compound No. 166)

### Example 2d

### Synthesis of hydrochloride salt of N-isopropyl-4-[6-(2-methylphenyl)-7-oxo-2-(piperidin-4-ylamino)pyrido[2,3-d]pyrimidin-8(7H)-yl]piperidine-1-carboxamide (Compound No. 135)

The title compound was prepared following the procedure as described in Example 2 by using the Compound No. 134 in place of Compound No. 1.

¹H NMR (CDCl₃+CD₃OD): δ 8.66 (s, 1H), 7.62-7.17 (m, 4H), 5.58-5.52 (m, 1H), 4.25-4.22 (m, 1H), 3.92-3.88 (m, 1H), 3.52-3.49 (m, 2H), 3.26-3.23 (m, 2H), 2.96-2.81 (m, 4H), 2.31-2.26 (m, 2H), 2.21 (s, 1H), 2.11-2.08 (m, 2H), 1.81-1.76 (m, 2H), 1.20-1.18 (m, 6H).

Mass (+ve ion mode m/z): 504 (M⁺+1).

The following analog can be prepared similarly using the appropriate corresponding starting materials:
Hydrochloride salt of 6-(2-methylphenyl)-8-[1-(methylsulfonyl)piperidin-4-yl]-2-(piperidin-4-ylamino)pyrido[2,3-d]pyrimidin-7(8H)-one (Compound No. 153)

### Example 2e

### Synthesis of N-isopropyl-4-[2-({1-[(isopropylamino)carbonyl]piperidin-4-yl}amino)-6-(2-methylphenyl)-7-oxopyrido[2,3-d]pyrimidin-8(7H)-yl]piperidine-1-carboxamide (Compound No. 136)

Step a: N-Isopropyl-4-[6-(2-methylphenyl)-7-oxo-2-(piperidin-4-ylamino)pyrido[2,3-d]pyrimidin-8(7H)-yl]piperidine-1-carboxamide

To a solution of the Compound No. 135 (1.7 g, 3.15 mmol) in dichloromethane was added triethylamine (0.96 g, 9.45 mmol) and isopropyl isocyanate (0.30 g, 3.46 mmol) and the mixture was stirred at room temperature for 2 hours. The reaction mixture was diluted with dichloromethane, washed with water and brine, dried over anhydrous sodium sulphate and concentrated under reduced pressure to yield the product. Yield: 1.6 g

¹H NMR (CDCl₃+CD₃OD): δ 8.58 (s, 1H), 7.31-7.17 (m, 4H), 5.58-5.54 (m, 1H), 4.28-4.24 (m, 1H), 4.04-4.02 (m, 2H), 3.91-3.89 (m, 2H), 3.94-3.77 (m, 2H), 2.94-2.87 (m, 8H), 2.20 (s, 3H), 1.78-1.61 (m, 4H), 1.16-1.11 (m, 12H).

Mass (+ve ion mode m/z): 589 (M⁺+1).

The compounds described below can be prepared by using appropriate corresponding reactants, for example, acyl halide, alkyl halide, sulphonyl halide, isocyanate or isothiocyanate in place of acetic anhydride, respectively, as applicable in each case:
4-[2-({1-[(Cyclopropylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 137)
4-[2-({1-[(tert-Butylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 138)
4-[2-({1-[(Cyclohexylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 139)
N-Isopropyl-4-[6-(2-methylphenyl)-2-{[1-(morpholin-4-ylcarbonyl)piperidin-4-yl]amino}-7-oxopyrido[2,3-d]pyrimidin-8(7H)-yl]piperidine-1-carboxamide (Compound No. 140),
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 141)
N-Isopropyl-4-[6-(2-methylphenyl)-2-{[1-(methylsulfonyl)piperidin-4-yl]amino}-7-oxopyrido[2,3-d]pyrimidin-8(7H)-yl]piperidine-1-carboxamide (Compound No. 142)
4-[2-{[1-(Ethylsulfonyl)piperidin-4-yl]amino}6-(2-methylphenyl)-7-oxopyrido[2,3-d]pyrimidin-8(7H)-yl]-N-isopropylpiperidine-1-carboxamide (Compound No. 143)
N-Isopropyl-4-[2-{[1-(isopropylsulfonyl)piperidin-4-yl]amino}-6-(2-methylphenyl)-7-oxopyrido[2,3-d]pyrimidin-8(7H)-yl]piperidine-1-carboxamide (Compound No. 144)
4-[2-{[1-(Cyclopropylcarbonyl)piperidin-4-yl]amino}-6-(2-methylphenyl)-7-oxopyrido[2,3-d]pyrimidin-8(7H)-yl]-N-isopropylpiperidine-1-carboxamide (Compound No. 145)
4-[2-[(1-Benzoylpiperidin-4-yl)amino]-6-(2-methylphenyl)-7-oxopyrido[2,3-d]pyrimidin-8(7H)-yl]-N-isopropylpiperidine-1-carboxamide (Compound No. 146)
4-[2-[(1-Acetylpiperidin-4-yl)amino]-6-(2-methylphenyl)-7-oxopyrido[2,3-d]pyrimidin-8(7H)-yl]-N-isopropylpiperidine-1-carboxamide (Compound No. 147)
N-Isopropyl-4-[6-(2-methylphenyl)-2-[(1-methylpiperidin-4-yl)amino]-7-oxopyrido[2,3-d]pyrimidin-8(7H)-yl]piperidine-1-carboxamide (Compound No. 148)
4-[2-[(1-Benzylpiperidin-4-yl)amino]-6-(2-methylphenyl)-7-oxopyrido[2,3-d]pyrimidin-8(7H)-yl]-N-isopropylpiperidine-1-carboxamide (Compound No. 149)
8-(1-Acetylpiperidin-4-yl)-2-[(1-acetylpiperidin-4-yl)amino]-6-(2-methylphenyl)pyrido[2,3-d]pyrimidin-7(8H)-one (Compound No. 150)
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 151)
N-Isopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 154)
N-(tert-Butyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 155)
N-Cyclohexyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 156)
N-(4-Fluorophenyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 157)
6-(2-Methylphenyl)-8-[1-(methylsulfonyl)piperidin-4-yl]-2-{[1-(morpholin-4-ylcarbonyl)piperidin-4-yl]amino}pyrido[2,3-d]pyrimidin-7(8H)-one (Compound No. 158)
6-(2-Methylphenyl)-8-[1-(methylsulfonyl)piperidin-4-yl]-2-{[1-(methylsulfonyl)piperidin-4-yl]amino}pyrido[2,3-d]pyrimidin-7(8H)-one (Compound No. 159),
2-{[1-(Ethylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 160),
2-{[1-(Isopropylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 161),
2-{[1-(Cyclopropylcarbonyl)piperidin-4-yl]amino}-6-(2-methylphenyl)-8-[1-(methylsulfonyl)piperidin-4-yl]pyrido[2,3-d]pyrimidin-7(8H)-one (Compound 162),
2-[(1-Acetylpiperidin-4-yl)amino]-6-(2-methylphenyl)-8-[1-(methylsulfonyl)piperidin-4-yl]pyrido[2,3-d]pyrimidin-7(8H)-one (Compound No. 163),
2-[(1-Benzoylpiperidin-4-yl)amino]-6-(2-methylphenyl)-8-[1-(methylsulfonyl)piperidin-4-yl]pyrido[2,3-d]pyrimidin-7(8H)-one (Compound No. 164),
2-{[1-(4-Fluorobenzoyl)piperidin-4-yl]amino}-6-(2-methylphenyl)-8-[1-(methylsulfonyl)piperidin-4-yl]pyrido[2,3-d]pyrimidin-7(8H)-one (Compound No. 165),
4-{[8-(1-Acetylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 168)
4-{[8-(1-Acetylpiperidin-4-yl)-6-(2-methylphenyl)-7-oxo-7,8-dihydropyrido[2,3-d]pyrimidin-2-yl]amino}-N-cyclopropylpiperidine-1-carboxamide (Compound No. 169)
4-{[8-(1-Acetylpiperidin-4-yl)-6-(2-methylphenyl)-7-oxo-7,8-dihydropyrido[2,3-d]pyrimidin-2-yl]amino}-N-(tert-butyl)piperidine-1-carboxamide (Compound No. 170)
4-{[8-(1-Acetylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 171)
4-{[8-(1-Acetylpiperidin-4-yl)-6-(2-methylphenyl)-7-oxo-7,8-dihydropyrido[2,3-d]pyrimidin-2-yl]amino}-N-(4-fluorophenyl)piperidine-1-carboxamide (Compound No. 172)
8-(1-Acetylpiperidin-4-yl)-6-(2-methylphenyl)-2-{[1-(morpholin-4-ylcarbonyl)piperidin-4-yl]amino}pyrido[2,3-d]pyrimidin-7(8H)-one (Compound No. 173)
8-(1-Acetylpiperidin-4-yl)-6-(2-methylphenyl)-2-{[1-(methylsulfonyl)piperidin-4-yl]amino}pyrido[2,3-d]pyrimidin-7(8H)-one (Compound No. 174)
8-(1-Acetylpiperidin-4-yl)-2-{[1-(ethylsulfonyl)piperidin-4-yl]amino}-6-(2-methylphenyl)pyrido[2,3-d]pyrimidin-7(8H)-one (Compound No. 175)
8-(1-Acetylpiperidin-4-yl)-2-{[1-(isopropylsulfonyl)piperidin-4-yl]amino}-6-(2-methylphenyl)pyrido[2,3-d]pyrimidin-7(8H)-one (Compound No. 176)
8-(1-Acetylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 177)
8-(1-Acetylpiperidin-4-yl)-2-{[1-(4-fluorobenzoyl)piperidin-4-yl]amino}-6-(2-methylphenyl)pyrido[2,3-d]pyrimidin-7(8H)-one (Compound No. 178)
4-[2-[(1-{[(4-Fluorophenyl)amino]carbonyl}piperidin-4-yl)amino]-6-(2-methylphenyl)-7-oxopyrido[2,3-d]pyrimidin-8(7H)-yl]-N-isopropylpiperidine-1-carboxamide (Compound No. 189)
4-{[8-{1-[(Isopropylamino)carbonyl]piperidin-4-yl}-6-(2-methylphenyl)-7-oxo-7,8-dihydropyrido[2,3-d]pyrimidin-2-yl]amino}-N-morpholin-4-ylpiperidine-1-carboxamide (Compound No. 190)
4-[2-{[1-(2,2-Dimethylpropanoyl)piperidin-4-yl]amino}-6-(2-methylphenyl)-7-oxopyrido[2,3-d]pyrimidin-8(7H)-yl]-N-isopropylpiperidine-1-carboxamide (Compound No. 191)
N-Cyclopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 193)
4-({6-(2-Methylphenyl)-8-[1-(methylsulfonyl)piperidin-4-yl]-7-oxo-7,8-dihydropyrido[2,3-d]pyrimidin-2-yl}amino)-N-morpholin-4-ylpiperidine-1-carboxamide (Compound No. 194)
2-{[1-(2,2-Dimethylpropanoyl)piperidin-4-yl]amino}-6-(2-methylphenyl)-8-[1-(methylsulfonyl)piperidin-4-yl]pyrido[2,3-d]pyrimidin-7(8H)-one (Compound No. 195)
6-(2-Methylphenyl)-2-[(1-methylpiperidin-4-yl)amino]-8-[1-(methylsulfonyl)piperidin-4-yl]pyrido[2,3-d]pyrimidin-7(8H)-one (Compound No. 196)
2-[(1-Benzylpiperidin-4-yl)amino]-6-(2-methylphenyl)-8-[1-(methylsulfonyl)piperidin-4-yl]pyrido[2,3-d]pyrimidin-7(8H)-one (Compound No. 197)
8-(1-Acetylpiperidin-4-yl)-2-[(1-benzoylpiperidin-4-yl)amino]-6-(2-methylphenyl)pyrido[2,3-d]pyrimidin-7(8H)-one (Compound No. 198)
8-(1-Acetylpiperidin-4-yl)-2-{[1-(2,2-dimethylpropanoyl)piperidin-4-yl]amino}-6-(2-methylphenyl)pyrido[2,3-d]pyrimidin-7(8H)-one (Compound No. 199)
8-(1-Acetylpiperidin-4-yl)-6-(2-methylphenyl)-2-[(1-methylpiperidin-4-yl)amino]pyrido[2,3-d]pyrimidin-7(8H)-one (Compound No. 200)
8-(1-Acetylpiperidin-4-yl)-2-[(1-benzylpiperidin-4-yl)amino]-6-(2-methylphenyl)pyrido[2,3-d]pyrimidin-7(8H)-one (Compound No. 201)

### Example 2f

### Synthesis of 2-[(1-acetylpiperidin-4-yl)amino]-6-(2-methylphenyl)-8-(1-methylpiperidin-4-yl)pyrido[2,3-d]pyrimidin-7(8H)-one (Compound No. 182)

To a solution of the Compound No. 113 (0.1 g, 0.23 mmol) in pyridine (2 mL) was added acetyl chloride (0.018 g, 0.23 mmol) and the mixture was stirred the reaction mixture at room temperature for 2 hours. The mixture was diluted with ethyl acetate, washed with water, brine, dried over anhydrous sodium sulphate and concentrated under reduced pressure to yield the title compound. Yield: 0.048 g.

¹H NMR (400 MHz, CD₃OD): δ 8.45 (s, 1H), 7.52-7.15 (m, 4H), 5.60-5.54 (m, 1H), 4.32-4.28 (m, 2H), 4.17-4.14 (m, 2H), 3.64-3.57 (m, 2H), 3.18-3.14 (m, 2H), 2.93-2.89 (m, 2H), 2.83 (s, 3H), 2.20 (s, 3H), 1.77-1.67 (m, 9H), 1.43-1.39 (m, 4H).

Mass (+ve ion mode m/z): 475 (M⁺+1).

Scheme I, Route II:

### Example 3

### Synthesis of tert-butyl 4-({6-(2-methylphenyl)-8-[(3S)-1-(methylsulfonyl)pyrrolidin-3-yl]-7-oxo-7,8-dihydropyrido[2,3-d]pyrimidin-2-yl}amino)piperidine-1-carboxylate (Compound No. 77)

Step a: Ethyl 4-amino-2-(methylthio)pyrimidine-5-carboxylate

To a suspension of ethyl-4-chloro-2-methylthio-5-pyrimidine-carboxylate (commercially available) (20.0 g, 85.9 mmol) in dry tetrahydrofuran (150 mL) was added triethylamine (13.01 g, 128.9 mmol) and aqueous ammonia (25%, 17.5 g, 258.2 mmol) at 0° C. and the mixture was stirred for 2 hours at room temperature. The organic solvent was evaporated under reduced pressure followed by addition of water. The residue thus separated was filtered and dried under reduced pressure to yield the title compound. Yield=17.0 g.

Step b: [4-Amino-2-(methylthio)pyrimidin-5-yl]methanol

To a suspension of lithium aluminium hydride (5.6 g, 150.2 mmol) in dry tetrahydrofuran (150 mL) at −70° C. was added dropwise a solution of the compound obtained from step a above (16.0 g, 75.1 mmol) in tetrahydrofuran (70 mL). The reaction mixture was stirred between −70° C. to −60° C. for 1 hour and then at room temperature until completion. The reaction mixture was cooled to 0° C. and diluted with ethyl acetate, followed by addition of 30% aqueous solution of sodium sulphate. The reaction mixture was then filtered through a celite pad and washed with ethyl acetate. The filtrate was extracted with ethyl acetate and the organic layer was washed with water, dried over anhydrous sodium sulphate, filtered and evaporated under reduced pressure to yield the title compound as a solid. Yield=14.0 g.

Step c: 4-Amino-2-(methylthio)pyrimidine-5-carbaldehyde

To a suspension of compound obtained from step b above (13.0 g, 76.02 mmol) in dichloromethane (200 mL) was added manganese dioxide (32.7 g, 380.1 mmol) at room temperature and the mixture was stirred for 24-36 hours. The reaction mixture was filtered over a celite pad and evaporated under reduced pressure to yield the title compound. Yield=11.0 g.

Step d: 6-(2-Methylphenyl)-2-(methylthio)pyrido[2,3-d]pyrimidin-7(8H)-one

To a solution of the compound obtained from step c above (10.0 g, 59.2 mmol) in N-methylpyrrolidinone (70 mL) was added 2-methylphenyl acetic acid methyl ester (10.6 g, 65.08 mmol) and potassium carbonate (24.5 g, 177.5 mmol) and the mixture was heated at 110° C. for 6-8 hours. The reaction mixture was diluted with ethyl acetate and poured into water. It was then extracted with ethyl acetate, washed with water, dried over anhydrous sodium sulphate, filtered and evaporated under reduced pressure. The residue thus obtained was purified by column chromatography using ethyl acetate in hexane (1:1) as eluent to yield the title compound. Yield=7.0 g.

Step e: tert-Butyl (3S)-3-[6-(2-methylphenyl)-2-(methylthio)-7-oxopyrido[2,3-d]pyrimidin-8(7H)-yl]pyrrolidine-1-carboxylate

To a solution of compound obtained from step d above (0.500 g, 1.76 mmol) in dry dimethylformamide (5 mL) was added triphenyl phosphine (0.92 g, 3.5 mmol) and R(-)-3-hydroxy-N—BOC pyrrolidine (0.36 g, 1.94 mmol). The reaction mixture was cooled to 0° C. and added dropwise diisopropyl azadicarboxylate (0.72 g, 3.5 mmol). The reaction mixture was stirred at room temperature for 3 hours. The reaction mixture was diluted with ethyl acetate and poured into water. The mixture was extracted with ethyl acetate, washed with water, dried over anhydrous sodium sulphate, filtered and evaporated under reduced pressure. The residue thus obtained was purified by column chromatography using ethyl acetate in hexane (25:75) as eluent to yield the title compound. Yield=0.450 g.

Step f: 6-(2-Methylphenyl)-2-(methylthio)-8-[(3S)-pyrrolidin-3-yl]pyrido[2,3-d]pyrimidin-7(8H)-one Hydrochloride

To a solution of compound obtained from step e above (0.400 g, 0.88 mmol) in dichloromethane (5 mL) was added methanolic HCl (4 N, 0.5 mL) at 0° C. and stirred at room temperature for 2 hours. The solvent was evaporated under reduced pressure to yield the title compound. Yield=0.350 g

Step g: 6-(2-Methylphenyl)-8-[(3S)-1-(methylsulfonyl)pyrrolidin-3-yl]-2-(methylthio)pyrido[2,3-d]pyrimidin-7(8H)-one

To a solution of compound obtained from step f above (0.350 g, 0.90 mmol) in pyridine (0.5 mL) was added methane sulphonyl chloride (0.123 g, 1.08 mmol) at 0° C. The reaction mixture was stirred at room temperature for 1 hour. Aqueous sodium bicarbonate solution was added to reaction mixture and then diluted with ethyl acetate. The mixture was extracted with ethyl acetate. The organic layer was collected, dried over anhydrous sodium sulphate and evaporated under reduced pressure. The crude product thus obtained was purified by column chromatography using ethyl acetate in hexane (8:2) as eluent to yield the title compound. Yield=0.250 g.

Step h: 6-(2-Methylphenyl)-2-(methylsulfonyl)-8-[(3S)-1-(methylsulfonyl)pyrrolidin-3-yl]pyrido[2,3-d]pyrimidin-7(8H)-one

To a solution of the compound obtained from step g above (0.250 g, 0.58 mmol) in chloroform (10 mL) was added m-chloroperbenzoic acid (77%, 0.390 g, 1.74 mmol) at 0° C. and the mixture was stirred at room temperature for 30 minutes. To the resulting reaction mixture was added a saturated solution of aqueous sodium bisulphite followed by aqueous sodium bicarbonate solution at 0° C. The reaction mixture was extracted with dichloromethane and the organic layer was washed with water, dried over anhydrous sodium sulphate, filtered and evaporated under reduced pressure. The residue thus obtained was washed thoroughly with hexane to yield the title compound. Yield=0.200 g

Step i: tert-Butyl 4-({6-(2-methylphenyl)-8-[(3S)-1-(methylsulfonyl)pyrrolidin-3-yl]-7-oxo-7,8-dihydropyrido[2,3-d]pyrimidin-2-yl}amino)piperidine-1-carboxylate

To the compound obtained from step h above (0.250 g, 0.54 mmol) was added tert-butyl 4-aminopiperidine-1-carboxylate (0.54 g, 2.7 mmol) and the mixture was stirred the reaction mixture at 65° C. for 2 hours. The reaction mixture was diluted with ethyl acetate. The reaction mixture was poured into water and extracted with ethyl acetate. The organic layer was separated, dried over anhydrous sodium sulphate and filtered. The filtrate was concentrated under reduced pressure and the residue thus obtained was purified by column chromatography using ethyl acetate in hexane (9:1) solvent mixture as eluent to yield the title compound. Yield=0.220 g.

¹H NMR (400 MHz, CD₃OD): δ 8.97 (s, 1H, Ar—H), 7.95 (s, 1H, ArH), 7.70-7.68 (m, 2H, Ar—H), 7.30-7.18 (m, 3H, Ar—H), 5.81 (s, 1H), 3.92-3.90 (m, 2H), 3.62-3.29 (m, 3H), 3.02-3.00 (m, 4H), 2.38 (s, 3H), 2.24-2.06 (m, 7H), 1.90-1.87 (m, 2H), 1.42 (s, 9H).

Mass (+ve ion mode m/z): 583 (M⁺+1).

The following analog can be prepared similarly by using by using acetic anhydride in place of using methane sulphonyl halide:
tert-Butyl 4-{[8-[(3R)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 184)

### Example 3a

### Synthesis of Hydrochloride Salt of 6-(2-methylphenyl)-8-[(3S)-1-(methylsulfonyl)pyrrolidin-3-yl]-2-(piperidin-4-ylamino)pyrido[2,3-d]pyrimidin-7(8H) one (Compound No. 183)

The title compound was prepared following the procedure as described in Example 2 by using the Compound No. 77 in place of Compound No. 1.

¹H NMR (400 MHz, CD₃OD): δ 9.43 (s, 1H, Ar—H), 8.27 (s, 1H, Ar—H), 7.35-7.20 (m, 4H, Ar—H), 5.91 (s, 1H), 4.62 (s, 1H), 3.62-3.31 (m, 4H), 3.30-3.02 (m, 4H), 2.34 (s, 3H), 2.33-2.28 (m, 4H), 2.17 (s, 3H), 2.15-1.98 (m, 2H)

Mass (+ve ion mode m/z): 483 (M⁺+1).

The following analog can be prepared similarly by using the appropriate corresponding starting materials:
Hydrochloride salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 81)

### Example 3b

### Synthesis of tert-butyl 4-({6-(2-methylphenyl)-7-oxo-8-[(3S -tetrahydrofuran-3-yl]-7,8-dihydropyrido[2,3-d]pyrimidin-2-yl}amino)piperidine-1-carboxylate (Compound No. 43)

Step a: 6-(2-Methylphenyl)-2-(methylthio)-8-[(3S)-tetrahydrofuran-3-yl]pyrido[2,3-d]pyrimidin-7(8H)-one

To a solution of compound 6-(2-methylphenyl)-2-(methylthio)pyrido[2,3-d]pyrimidin-7(8H)-one (Example 3, step d) (2.0 g, 7.06 mmol) in dry dimethyl formamide (10 mL) was added triphenyl phosphine (3.7 g, 14.1 mmol) and R(-)-3-hydroxy tetrahydrofuran (0.68 g, 7.7 mmol). The mixture was cooled to 0° C. and diisopropyl azadicarboxylate (2.85 g, 14.1 mmol) was added dropwise. The reaction mixture was stirred at room temperature for 3 hours, diluted with ethyl acetate and poured into water. The mixture was then extracted with ethyl acetate, washed with water, dried over anhydrous sodium sulphate, filtered and evaporated under reduced pressure. The residue thus obtained was purified by column chromatography using ethyl acetate in hexane (3:7) as eluent to yield the title compound. Yield=2.0 g.

Step b: 6-(2-Methylphenyl)-2-(methylsulfonyl)-8-[(3S)-tetrahydrofuran-3-yl]pyrido[2,3-d]pyrimidin-7(8H)-one

To a solution of the compound obtained from step a above (2.0 g, 5.6 mmol) in chloroform (20 mL) was added m-chloroperbenzoic acid (77%) (3.8 g, 16.9 mmol) at 0° C. and the mixture was stirred at room temperature for 30 minutes. To the mixture was added a saturated solution of aqueous sodium bisulphite followed by aqueous sodium bicarbonate solution at 0° C. The reaction mixture was then extracted with dichloromethane and the organic layer was washed with water, dried over anhydrous sodium sulphite, filtered and evaporated under reduced pressure. The residue thus obtained was washed thoroughly with hexane to yield the title compound. Yield=2.0 g

Step c: tert-Butyl 4-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate

To the compound obtained from step b above (0.7 g, 1.8 mmol) was added 4-amino-N—BOC piperidine (1.09 g, 5.45 mmol) and the mixture was heated to 65° C. for 2 hours. The reaction mixture was diluted with ethyl acetate and the compound was purified by column chromatography using ethyl acetate in hexane (9:1) solvent mixture as eluent to yield the title compound. Yield=260 mg.

¹H NMR (400 MHz, CDCl₃+CD₃OD): δ 8.84 (s, 1H (ArH), 7.77 (s, 1H (ArH), 7.30-7.24 (m, 3H, Ar—H), 7.17-7.15 (m, 1H, Ar—H), 5.88 (s, 1H), 4.24 (s, 1H), 4.12-4.09 (m, 4H), 3.93-3.80 (m, 4H), 2.16 (s, 3H), 2.12-2.05 (m, 3H), 1.49 (s, 9H).

Mass (+ve ion mode m/z): 506 (M⁺+1).

The following analogs can be prepared similarly using the appropriate corresponding starting materials:
tert-Butyl 4-{[6-(2-methylphenyl)-7-oxo-8-(tetrahydrofuran-3-yl)-7,8-dihydropyrido[2,3-d]pyrimidin-2-yl]amino}piperidine-1-carboxylate (Compound No. 36)
tert-Butyl 4-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 48)
tert-Butyl (3S)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 70)
tert-Butyl (3S)-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate (Compound No. 72)
tert-Butyl (3R)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 73)
tert-Butyl (3S)-3-{[6-(2-methylphenyl)-7-oxo-8-(tetrahydrofuran-3-yl)-7,8-dihydropyrido[2,3-d]pyrimidin-2-yl]amino}pyrrolidine-1-carboxylate (Compound No. 103)

### Example 3c

### Synthesis of Hydrochloride Salt 6-(2-methylphenyl)-2-(piperidin-4-ylamino)-8-[(3S)-tetrahydrofuran-3-yl]pyrido[2,3-d]pyrimidin-7(8H)-one (Compound No. 50)

The title compound was prepared following the procedure as described in Example 2 by using the Compound No. 43 in place of Compound No. 1.

¹H NMR (400 MHz, CD₃OD): δ 8.16 (s, 1H, Ar—H), 7.62-7.19 (m, 5H, Ar—H), 5.83 (s, 1H), 4.09-4.06 (m, 1H), 4.04-4.00 (m, 2H), 3.90-3.77 (m, 4H), 3.54-3.51 (m, 2H), 3.23-3.20 (m, 2H), 2.34-2.31 (m, 2H), 2.16 (s, 3H), 2.06-2.01 (m, 2H).

Mass (+ve ion mode m/z): 406 (M⁺+1).

The following analogs were prepared similarly using the appropriate corresponding starting materials:
Hydrochloride salt of 6-(2-methylphenyl)-2-(piperidin-4-ylamino)-8-[(3R)-tetrahydrofuran-3-yl]pyrido[2,3-d]pyrimidin-7(8H)-one (Compound No. 49)
Hydrochloride salt of 6-(2-methylphenyl)-2-[(3S)-pyrrolidin-3-ylamino]-8-(tetrahydrofuran-3-yl)pyrido[2,3-d]pyrimidin-7(8H)-one (Compound No. 185)
Hydrochloride salt of 6-(2-methylphenyl)-2-[(3S)-piperidin-3-ylamino]-8-[(3R)-tetrahydrofuran-3-yl]pyrido[2,3-d]pyrimidin-7(8H)-one (Compound No. 186)
Hydrochloride salt of 6-(2-methylphenyl)-2-[(3S)-piperidin-3-ylamino]-8-[(3S)-tetrahydrofuran-3-yl]pyrido[2,3-d]pyrimidin-7(8H)-one (Compound No. 187)

Hydrochloride salt of 6-(2-methylphenyl)-2-[(3R)-piperidin-3-ylamino]-8-[(3R)-tetrahydrofuran-3-yl]pyrido[2,3-d]pyrimidin-7(8H)-one (Compound No. 188)

### Example 4

### Synthesis of tert-butyl 4-{[8-[(3S)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 179)

Step a: 8-[(3S)-1-Acetylpyrrolidin-3-yl]-6-(2-methylphenyl)-2-(methylthio)pyrido[2,3-d]pyrimidin-7(8H)-one

To a solution of compound hydrochloride salt of 6-(2-methylphenyl)-2-(methylthio)-8-[(3S)-pyrrolidin-3-yl]pyrido[2,3-d]pyrimidin-7(8H)-one hydrochloride obtained from Example 3, step f (0.400 g, 1.03 mmol) in pyridine (5.0 mL) was added acetic anhydride (5 mL) at 0° C. The reaction mixture was stirred at room temperature for 1 hour. The reaction mixture was diluted with ethyl acetate and added water. The compound was extracted with ethyl acetate. The organic layer was collected, dried over anhydrous sodium sulphate and evaporated under reduced pressure to yield the crude product. The crude product obtained was purified by column chromatography using ethyl acetate in hexane (8:2) as eluent to yield the title compound. Yield=0.500 g

Step b: 8-[(3S)-1-Acetylpyrrolidin-3-yl]-6-(2-methylphenyl)-2-(methylsulfonyl)pyrido[2,3-d]pyrimidin-7(8H)-one

To a solution of the compound obtained from step a above (0.380 g, 0.96 mmol) in chloroform (10 mL) was added m-chloroperbenzoic acid (77%) (0.650 g, 2.89 mmol) at 0° C. and the mixture was stirred at room temperature for 30 minutes. To the mixture was added a saturated solution of aqueous sodium bisulphate followed by aqueous sodium bicarbonate solution at 0° C. The reaction mixture was then extracted with dichloromethane and the organic layer was washed with water, dried over anhydrous sodium sulphate, filtered and evaporated under reduced pressure. The residue thus obtained was washed thoroughly with hexane to yield the title compound. Yield=0.420 g

Step c: tert-Butyl 4-({8-[(3S)-1-acetylpyrrolidin-3-yl]-6-(2-methylphenyl)-7-oxo-7,8-dihydropyrido[2,3-d]pyrimidin-2-yl}-amino)piperidine-1-carboxylate

To the compound obtained from step b above (0.400 g, 0.93 mmol) was added tert-butyl 4-aminopiperidine-1-carboxylate (0.56 g, 2.8 mmol), pyridine (1 mL) and the mixture was heated to 65° C. for 2 hours. The reaction mixture was poured in water and extracted with ethyl acetate. The organic layer was collected, dried over anhydrous sodium sulphate and evaporated under reduced pressure to obtain the crude product. The crude compound was purified by column chromatography using ethyl acetate in hexane (9:1) solvent mixture as eluent to yield the title compound. Yield=0.100 g.

¹H NMR (400 MHz, CD₃Cl₃): δ 8.45 (s, 1H, Ar—H), 7.43 (1H, Ar—H), 7.29-7.16 (m, 4H, Ar—H), 6.29 (s, 1H), 4.05-3.94 (m, 6H), 3.02-2.97 (m, 2H), 2.22 (s, 3H), 2.20 (s, 1H), 2.17 (s, 3H), 2.09-2.04 (m, 3H), 1.47 (s, 9H), 1.45 (s, 2H)

Mass (+ve ion mode m/z): 547 (M⁺+1).

### Example 4a

### Synthesis of Hydrochloride Salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 180)

The title compound was prepared following the procedure as described in Example 2 by using the Compound No. 179 in place of Compound No. 1.

¹H NMR (400 MHz, CD₃Cl₃): δ 8.68 (s, 1H, Ar—H), 7.71 (s, 1H, Ar—H), 7.29-7.16 (m, 4H, Ar—H), 5.75 (s, 1H), 3.62-3.02 (m, 7H), 2.58-2.48 (m, 2H), 2.18 (s, 3H), 2.15 (s, 3H), 2.06-1.83 (m, 4H), 1.16-1.10 (m, 2H).

Mass (+ve ion mode m/z): 447 (M⁺+1).

### Example 4b

### Synthesis of 4-({8-[(3S)-1-acetylpyrrolidin-3-yl]-6-(2-methylphenyl)-7-oxo-7,8-dihydropyrido[2,3-d]pyrimidin-2-yl}amino)-N-isopropylpiperidine-1-carboxamide (Compound No. 80)

The title compound was prepared following the procedure as described in Example 5 by using the Compound No. 180 in place of Compound No. 2.

¹H NMR (400 MHz, CD₃OD): δ 8.91 (s, 1H, Ar—H), 7.77 (s, 1H, Ar—H), 7.43-6.99 (m, 4H, Ar—H), 5.94 (s, 1H), 4.27-4.25 (m, 1H), 4.00-3.88 (m, 4H), 3.78-3.73 (m, 2H), 3.60-3.58 (m, 1H), 3.09-3.06 (m, 2H), 2.24-2.23 (m, 1H), 2.21 (s, 3H), 2.20 (s, 3H), 2.19-2.18 (m, 1H), 1.67-1.65 (m, 2H), 1.25 (s, 2H), 1.17-1.13 (m, 6H).

Mass (+ve ion mode m/z): 532 (M⁺+1).

The following analogs can be prepared by using appropriate corresponding compound, for example, acyl halide, alkyl halide, sulphonyl halide, isocyanate or isothiocyanate in place of acetic anhydride, respectively, as applicable in each case.
6-(2-Methylphenyl)-2-{[1-(methylsulfonyl)piperidin-4-yl]amino}-8-[(3S)-1-(methylsulfonyl)pyrrolidin-3-yl]pyrido[2,3-d]pyrimidin-7(8H)-one (Compound No. 78)
8-[(3S)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 192)
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 202)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 203)
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 204)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(4-fluorophenyl)piperidine-1-carboxamide (Compound No. 205)
4-{[8-[(3S)-1-Acetylpyrrolidin-3-yl]-6-(2-methylphenyl)-7-oxo-7,8-dihydropyrido[2,3-d]pyrimidin-2-yl]amino}-N-morpholin-4-ylpiperidine-1-carboxamide (Compound No. 206)
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 181)
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 207)
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one ((Compound No. 208)
8-[(3R)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 209)
2-[(1-Acetylpiperidin-4-yl)amino]-8-[(3R)-1-acetylpyrrolidin-3-yl]-6-(2-methylphenyl)pyrido[2,3-d]pyrimidin-7(8H)-one (Compound No. 210)
8-[(3R)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 211)
8-[(3R)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 212)
8-[(3R)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 213)
8-[(3R)-1-Acetylpyrrolidin-3-yl]-6-(2-methylphenyl)-2-[(1-methylpiperidin-4-yl)amino]pyrido[2,3-d]pyrimidin-7(8H)-one (Compound No. 214)
8-[(3R)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 215)
8-[(3S)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 216)
8-[(3S)-1-Acetylpyrrolidin-3-yl]-6-(2-methylphenyl)-2-[(1-methylpiperidin-4-yl)amino]pyrido[2,3-d]pyrimidin-7(8H)-one (Compound No. 217)
4-{[8-[(3S)-1-Acetylpyrrolidin-3-yl]-6-(2-methylphenyl)-7-oxo-7,8-dihydropyrido[2,3-d]pyrimidin-2-yl]amino}-N-isopropylpiperidine-1-carboxamide (Compound No. 218)
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 219)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 220)
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 221)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(4-fluorophenyl)piperidine-1-carboxamide (Compound No. 222)
4-{[8-[(3S)-1-Acetylpyrrolidin-3-yl]-6-(2-methylphenyl)-7-oxo-7,8-dihydropyrido[2,3-d]pyrimidin-2-yl]amino}-N-morpholin-4-ylpiperidine-1-carboxamide (Compound No. 223)
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 224)
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 225)
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one (Compound No. 226)
8-[(3S)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 227)
2-[(1-Acetylpiperidin-4-yl)amino]-8-[(3S)-1-acetylpyrrolidin-3-yl]-6-(2-methylphenyl)pyrido[2,3-d]pyrimidin-7(8H)-one (Compound No. 228)
8-[(3S)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 229)
8-[(3S)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 230)

Scheme II:

### Example 5

### Synthesis of 2-[(1-acetylpiperidin-4-yl)amino]-6-(2-methylphenyl)-8-(tetrahydro-2H-pyran-4-yl)pyrido[2,3-d]pyrimidin-7(8H)-one (Compound No. 3)

To a solution of the Compound No. 2 (50 mg) in pyridine (2 mL) was added acetic anhydride (4 mL) at 0° C. and the mixture was stirred for 4 hours. To the reaction mixture was added water, which was then extracted with ethyl acetate. The organic layer was washed with water, dried over anhydrous sodium sulphate, filtered and evaporated under reduced pressure. The residue thus obtained was purified by preparative TLC and dried under vacuum to yield the title compound. Yield=28 mg.

¹H NMR (400 MHz, CDCl₃) δ: 8.43 (s, 1H, Ar—H), 7.41 (s, 1H, Ar—H), 7.31-7.17 (m, 4H, Ar—H), 5.65-5.56 (m, 2H, 1NH, 1N—CH), 4.61 (m, 1H, N—CH), 4.13-4.10 (m, 2H, —OCH₂), 3.90-3.86 (m, 2H, —OCH₂), 3.57-3.48 (m, 2H, N—CH₂), 3.25-3.13 (m, 2H, N—CH₂), 2.22 (s, 3H, Ar—CH₃+2H, —CH₂), 2.19-2.12 (m, 5H, —COCH₃+—CH₂), 1.73-1.57 (m, 4H).

Mass (+ve ion mode m/z): 462 (M⁺+1)

m.p: 235.1-236.6° C.

The analogs of 2-[(1-acetylpiperidin-4-yl)amino]-6-(2-methylphenyl)-8-(tetrahydro-2H-pyran-4-yl)pyrido[2,3-d]pyrimidin-7(8H)-one (Compound No. 3) described below can be prepared by using appropriate corresponding compound, for example, acyl halide, alkyl halide, sulphonyl halide, isocyanate or isothiocyanate in place of acetic anhydride, respectively, as applicable in each case.
6-(2-Methylphenyl)-2-{[1-(methylsulfonyl)piperidin-4-yl]amino}-8-(tetrahydro-2H-pyran-4-yl)pyrido[2,3-d]pyrimidin-7(8H)-one (Compound No. 4)
2-[(1-Benzoylpiperidin-4-yl)amino]-6-(2-methylphenyl)-8-(tetrahydro-2H-pyran-4-yl)pyrido[2,3-d]pyrimidin-7(8H)-one (Compound No. 5)
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 6)
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 7)
2-{[1-(Ethylsulfonyl)piperidin-4-yl]amino}-6-(2-methylphenyl)-8-(tetrahydro-2H-pyran-4-yl)pyrido[2,3-d]pyrimidin-7(8H)-one (Compound No. 8)
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 10)
4-{[6-(2-Methylphenyl)-7-oxo-8-(tetrahydro-2H-pyran-4-yl)-7,8-dihydropyrido[2,3-d]pyrimidin-2-yl]amino}-N-[4-(trifluoromethyl)phenyl]piperidine-1-carboxamide (Compound No. 11)
2-{[1-(Isopropylsulfonyl)piperidin-4-yl]amino}-6-(2-methylphenyl)-8-(tetrahydro-2H-pyran-4-yl)pyrido[2,3-d]pyrimidin-7(8H)-one (Compound No. 14)
4-{[6-(2-Methylphenyl)-7-oxo-8-(tetrahydro-2H-pyran-4-yl)-7,8-dihydropyrido[2,3-d]pyrimidin-2-yl]amino}-N-propylpiperidine-1-carboxamide (Compound No. 18)
N-[(1S)-1,2-Dimethylpropyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 19)
N-Cyclohexyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 20)
2-{[1-(4-Fluorobenzoyl)piperidin-4-yl]amino}-6-(2-methylphenyl)-8-(tetrahydro-2H-pyran-4-yl)pyrido[2,3-d]pyrimidin-7(8H)-one (Compound No. 21)
N-(Cyclopentylmethyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 22)
4-{[6-(2-Methylphenyl)-7-oxo-8-(tetrahydro-2H-pyran-4-yl)-7,8-dihydropyrido[2,3-d]pyrimidin-2-yl]amino}-N-(1,1,3,3-tetramethylbutyl)piperidine-1-carboxamide (Compound No. 23)
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carboxamide (Compound No. 24)
N-Cyclopentyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 25)
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 26)
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carbothioamide (Compound No. 27)
N-tert-Butyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 28)
6-(2-Methylphenyl)-2-[(1-pyrimidin-2-ylpiperidin-4-yl)amino]-8-(tetrahydro-2H-pyran-4-yl)pyrido[2,3-d]pyrimidin-7(8H)-one (Compound No. 29)
N-Cyclopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 30)
N-[(1R)-1-Cyclohexylethyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 31)
2-{[1-(Cyclopentylcarbonyl)piperidin-4-yl]amino}-6-(2-methylphenyl)-8-(tetrahydro-2H-pyran-4-yl)pyrido[2,3-d]pyrimidin-7(8H)-one (Compound No. 32)
6-(2-Methylphenyl)-2-{[1-(pyrrolidin-1-ylcarbonyl)piperidin-4-yl]amino}-8-(tetrahydro-2H-pyran-4-yl)pyrido[2,3-d]pyrimidin-7(8H)-one (Compound No. 33)
N-Isopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 37)
6-(2-Methylphenyl)-7-oxo-2-{[1-(pyrrolidin-1-ylcarbonyl)piperidin-4-yl]amino}-1-(tetrahydrofuran-3-yl)-7,8-dihydropyrido[2,3-d]pyrimidin-1-ium (Compound No. 38)
2-{[1-(Ethylsulfonyl)piperidin-4-yl]amino}-6-(2-methylphenyl)-7-oxo-1-(tetrahydrofuran-3-yl)-7,8-dihydropyrido[2,3-d]pyrimidin-1-ium (Compound No. 39)
6-(2-Methylphenyl)-2-{[1-(methylsulfonyl)piperidin-4-yl]amino}-8-(tetrahydrofuran-3-yl)pyrido[2,3-d]pyrimidin-7(8H)-one (Compound No. 40)
N-Cyclopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 41)
6-(2-Methylphenyl)-2-{[(3S)-1-(methylsulfonyl)pyrrolidin-3-yl]amino}-8-(tetrahydrofuran-3-yl)pyrido[2,3-d]pyrimidin-7(8H)-one (Compound No. 42)
6-(2-Methylphenyl)-2-{[1-(methylsulfonyl)piperidin-4-yl]amino}-8-[(3S)-tetrahydrofuran-3-yl]pyrido[2,3-d]pyrimidin-7(8H)-one (Compound No. 44)
N-Isopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 45)
6-(2-Methylphenyl)-2-{[1-(methylsulfonyl)piperidin-4-yl]amino}-8-[(3R)-tetrahydrofuran-3-yl]pyrido[2,3-d]pyrimidin-7(8H)-one (Compound No. 46)
N-Isopropyl-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 47)
2-{[1-(Ethylsulfonyl)piperidin-4-yl]amino}-6-(2-methylphenyl)-8-[(3S)-tetrahydrofuran-3-yl]pyrido[2,3-d]pyrimidin-7(8H)-one (Compound No. 51)
N-Cyclopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 52)
N-Cyclopropyl-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 53)
2-[(1-Benzylpiperidin-4-yl)amino]-6-(2-methylphenyl)-8-[(3R)-tetrahydrofuran-3-yl]pyrido[2,3-d]pyrimidin-7(8H)-one (Compound No. 54)
2-(Cyclobutylamino)-6-(2-methylphenyl)-8-[(3R)-tetrahydrofuran-3-yl]pyrido[2,3-d]pyrimidin-7(8H)-one (Compound No. 55)
2-(Cyclopropylamino)-6-(2-methylphenyl)-8-[(3R)-tetrahydrofuran-3-yl]pyrido[2,3-d]pyrimidin-7(8H)-one (Compound No. 56)
2-{[1-(Ethylsulfonyl)piperidin-4-yl]amino}-6-(2-methylphenyl)-8-[(3R)-tetrahydrofuran-3-yl]pyrido[2,3-d]pyrimidin-7(8H)-one (Compound No. 57)
2-[(1-Acetylpiperidin-4-yl)amino]-6-(2-methylphenyl)-8-[(3R)-tetrahydrofuran-3-yl]pyrido[2,3-d]pyrimidin-7(8H)-one (Compound No. 58)
2-{[1-(4-Fluorobenzoyl)piperidin-4-yl]amino}-6-(2-methylphenyl)-8-[(3R)-tetrahydrofuran-3-yl]pyrido[2,3-d]pyrimidin-7(8H)-one (Compound No. 59)
2-{[1-(Cyclopropylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 60)
2-{[1-(Cyclopropylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 61)
2-{[1-(Cyclopentylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 62),
2-{[1-(Cyclopentylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 63)
2-[(1-Benzoylpiperidin-4-yl)amino]-6-(2-methylphenyl)-8-[(3S)-tetrahydrofuran-3-yl]pyrido[2,3-d]pyrimidin-7(8H)-one (Compound No. 64)
2-[(1-Benzoylpiperidin-4-yl)amino]-6-(2-methylphenyl)-8-[(3R)-tetrahydrofuran-3-yl]pyrido[2,3-d]pyrimidin-7(8H)-one (Compound No. 65)
2-{[1-(4-Fluorobenzoyl)piperidin-4-yl]amino}-6-(2-methylphenyl)-8-[(3S)-tetrahydrofuran-3-yl]pyrido[2,3-d]pyrimidin-7(8H)-one (Compound No. 66)
N-(4-Fluorophenyl)-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 67)
2-[(1-Acetylpiperidin-4-yl)amino]-6-(2-methylphenyl)-8-[(3S)-tetrahydrofuran-3-yl]pyrido[2,3-d]pyrimidin-7(8H)-one (Compound No. 68)
N-(4-Fluorophenyl)-4-({6-(2-methylphenyl)-7-oxo-8-[(3S)-tetrahydro furan-3-yl]-7,8-dihydropyrido[2,3-d]pyrimidin-2-yl}amino)piperidine-1-carboxamide (Compound No. 69)
(3S)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 71)
(3R)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 74)
(3S)—N-Isopropyl-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 75)
6-(2-Methylphenyl)-2-{[(3S)-1-(methylsulfonyl)piperidin-3-yl]amino}-8-[(3S)-tetrahydrofuran-3-yl]pyrido[2,3-d]pyrimidin-7(8H)-one (Compound No. 76)
6-(2-Methylphenyl)-2-{[1-(methylsulfonyl)piperidin-4-yl]amino}-8-[(3S)-1-(methylsulfonyl)pyrrolidin-3-yl]pyrido[2,3-d]pyrimidin-7(8H)-one (Compound No. 77)
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 83)
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carbothioamide (Compound No. 84)
6-(2-Methylphenyl)-2-{[(3S)-1-(methylsulfonyl)pyrrolidin-3-yl]amino}-8-(tetrahydro-2H-pyran-4-yl)pyrido[2,3-d]pyrimidin-7(8H)-one (Compound No. 85)
2-{[(3S)-1-(Ethylsulfonyl)pyrrolidin-3-yl]amino}-6-(2-methylphenyl)-8-(tetrahydro-2H-pyran-4-yl)pyrido[2,3-d]pyrimidin-7(8H)-one (Compound No. 86)
2-{[(3S)-1-Acetylpyrrolidin-3-yl]amino}-6-(2-methylphenyl)-8-(tetrahydro-2H-pyran-4-yl)pyrido[2,3-d]pyrimidin-7(8H)-one (Compound No. 87)
(3S)—N-Cyclopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 88)
(3S)—N-Butyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 90)
(3S)—N-Cyclopentyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 91)
(3S)—N-[(1S)-1,2-Dimethylpropyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 92)
2-{[(3R)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 93)
2-{[(3S)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 94)
(3S)—N-Cyclohexyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 95)
(3S)-3-{[6-(2-Methylphenyl)-7-oxo-8-(tetrahydro-2H-pyran-4-yl)-7,8-dihydropyrido[2,3-d]pyrimidin-2-yl]amino}-N-octylpyrrolidine-1-carboxamide (Compound No. 96)
2-{[(3S)-1-(Cyclopropylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 97)
2-{[(3S)-1-(Cyclopentylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 98)
6-(2-Methylphenyl)-2-{[(3S)-1-pyrimidin-2-yl-pyrrolidin-3-yl]amino}-8-(tetrahydro-2H-pyran-4-yl)pyrido[2,3-d]pyrimidin-7(8H)-one (Compound No. 99)
(3S)—N-[(1R)-1-Cyclohexylethyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 100)
6-(2-Methylphenyl)-2-{[(3S)-1-(pyrrolidin-1-ylcarbonyl)pyrrolidin-3-yl]amino}-8-(tetrahydro-2H-pyran-4-yl)pyrido[2,3-d]pyrimidin-7(8H)-one (Compound No. 101)
2-{[(3S)-1-(Cyclopentylacetyl)pyrrolidin-3-yl]amino}-6-(2-methylphenyl)-8-(tetrahydro-2H-pyran-4-yl)pyrido[2,3-d]pyrimidin-7(8H)-one (Compound No. 102)
(3S)—N-Isopropyl-3-{[6-(2-methylphenyl)-7-oxo-8-(tetrahydrofuran-3-yl)-7,8-dihydropyrido[2,3-d]pyrimidin-2-yl]amino}pyrrolidine-1-carboxamide (Compound No. 105)
8-(1-Benzylpiperidin-4-yl)-6-(2-methylphenyl)-2-[(1-methylpiperidin-4-yl)amino]pyrido[2,3-d]pyrimidin-7(8H)-one (Compound No. 108)
4-{[8-(1-Benzylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 114)
4-{[8-(1-Benzylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 115)
4-{[8-(1-Benzylpiperidin-4-yl)-6-(2-methylphenyl)-7-oxo-7,8-dihydropyrido[2,3-d]pyrimidin-2-yl]amino}-N-cyclopentylpiperidine-1-carboxamide (Compound No. 116)
4-{[8-(1-Benzylpiperidin-4-yl)-6-(2-methylphenyl)-7-oxo-7,8-dihydropyrido[2,3-d]pyrimidin-2-yl]amino}-N-isobutylpiperidine-1-carboxamide (Compound No. 117)
4-{[8-(1-Benzylpiperidin-4-yl)-6-(2-methylphenyl)-7-oxo-7,8-dihydropyrido[2,3-d]pyrimidin-2-yl]amino}-N-[(1R)-1-cyclohexylethyl]piperidine-1-carboxamide (Compound No. 118)
N-Isopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 119),
N-Cyclopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 120)
8-(1-Benzylpiperidin-4-yl)-2-{[1-(cyclopentylacetyl)piperidin-4-yl]amino}-6-(2-methylphenyl)pyrido[2,3-d]pyrimidin-7(8H)-one (Compound No. 121)
8-(1-Benzylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 122)
8-(1-Benzylpiperidin-4-yl)-6-(2-methylphenyl)-2-{[1-(phenylsulfonyl)piperidin-4-yl]amino}pyrido[2,3-d]pyrimidin-7(8H)-one (Compound No. 123)
8-(1-Benzylpiperidin-4-yl)-6-(2-methylphenyl)-2-{[1-(methylsulfonyl)piperidin-4-yl]amino}pyrido[2,3-d]pyrimidin-7(8H)-one (Compound No. 124)
8-(1-Benzylpiperidin-4-yl)-2-{[1-(ethylsulfonyl)piperidin-4-yl]amino}-6-(2-methylphenyl)pyrido[2,3-d]pyrimidin-7(8H)-one (Compound no. 125)
6-(2-Methylphenyl)-8-(1-methylpiperidin-4-yl)-2-{[1-(methylsulfonyl)piperidin-4-yl]amino}pyrido[2,3-d]pyrimidin-7(8H)-one (Compound No. 126)
2-{[1-(Ethylsulfonyl)piperidin-4-yl]amino}-6-(2-methylphenyl)-8-(1-methylpiperidin-4-yl)pyrido[2,3-d]pyrimidin-7(8H)-one (Compound No. 127)
2-{[1-(Isopropylsulfonyl)piperidin-4-yl]amino}-6-(2-methylphenyl)-8-(1-methylpiperidin-4-yl)pyrido[2,3-d]pyrimidin-7(8H)-one (Compound No. 128)
6-(2-Methylphenyl)-8-(1-methylpiperidin-4-yl)-2-{[1-(pyrrolidin-1-ylcarbonyl)piperidin-4-yl]amino}pyrido[2,3-d]pyrimidin-7(8H)-one (Compound No. 129)
2-[(1-Benzoylpiperidin-4-yl)amino]-6-(2-methylphenyl)-8-(1-methylpiperidin-4-yl)pyrido[2,3-d]pyrimidin-7(8H)-one (Compound No. 130)

### Example 5a

### Synthesis of N-isopropyl-4-{[6-(2-methylphenyl)-7-oxo-8-piperidin-4-yl-7,8-dihydropyrido[2,3-d]pyrimidin-2-yl]amino}piperidine-1-carboxamide (Compound No. 133)

The title compound was prepared following the procedure as described in Example 2a by using the Compound No. 114 in place of Compound No. 106.

¹H NMR (400 MHz, CDCl₃) δ: 8.41 (s, 1H), 7.39 (s, 1H), 7.16-7.28, 5.38-5.44 (m, 2H),

(m, 3H), 4.06-4.28 (m, 4H), 3.93-3.96 (m, 4H), 2.95-3.01 (m, 4H), 2.21 (s, 3H), 2.09-2.17 (m, 2H), 1.47-1.63 (m, 6H), 1.19-1.25 (m, 6H).

Mass (+ve ion mode m/z): 504 (M⁺+1)

### Example 6

### p38 Inhibition Assays

Inhibition of Phosphorylation of EGF Receptor Peptide

This assay was carried out in the presence of 10 mM MgCl2, 25 mM -glycerophosphate, 10% glycerol and 100 mM HEPES buffer at pH 7.6. For a typical IC₅₀ determination, a stock solution was prepared containing all of the above components and activated p38 (5 nM). The stock solution was aliquoted into vials. A fixed volume of DMSO or inhibitor in DMSO (final concentration of DMSO in the reaction was 5%) was introduced to each vial, mixed and incubated for 15 minutes at room temperature.

EGF receptor peptide, KRELVEPLTPSGEAPNQALLR, a phosphoryl acceptor in p38-catalysed kinase reaction (1) was added to each vial to a final concentration of 200 μM. The kinase reaction was initiated with ATP (100 μm) and the vials were incubated at 30° C. After 30 minutes, the reactions were quenched with equal volume of 10% trifluoroacetic acid (TFA).

The phosphorylated peptide was quantified by HPLC analysis. Separation of the phosphorylated peptide from the unphosphorylated peptide was achieved on a reverse phase column (Deltapak, 5 μM, C18 100D, part no. 011795) with a binary gradient of water and acetonitrile, each containing 0.1% TFA. IC₅₀ (concentration of inhibitor yielding 50% inhibition) was determined by plotting the % activity remaining against inhibitor concentration.

Cell Based Assay for TNF-α Release

Method of Isolation of Human Peripheral Blood Mononuclear Cells:

Human whole blood was collected in vacutainer tubes containing EDTA as an anti coagulant. A blood sample (7 mL) was carefully layered over 5 mL PMN Cell Isolation Medium (Robbins Scientific) in a 15 mL round bottom centrifuge tubes. The sample was centrifuged at 450-500×g for 30-35 minutes in a swing out rotor at room temperature. After centrifugation the top band of cells were removed and washed 3 times with PBS w/o calcium or magnesium. The cells were centrifuged at 400×g for 10 minutes at room temperature. The cells were resuspended in Macrophage Serum Free Medium (Gibco BRL) at concentration of 2 million cells/mL.

LPS Stimulation of Human PBMNC's:

PBM cells (0.1 mL; 2 million/mL) were co-incubated with 0.1 mL of compound (10-0.41 μM, final concentration) for 1 hour in flat bottom 96 well microtiter plate. Compounds were dissolved in DMSO initially and diluted in TCM for a final concentration of 0.1% DMSO. LPS (Cal biochem, 20 ng/mL, final concentration) was then added at volume of 0.010 mL. Cultures were incubated overnight at 37° C.). Supernatant was then removed and tested by ELISA for TNF-α release. Viability was analyzed using MTT. After 0.1 mL supernatant was collected, 0.1 mL of 0.25 mg/mL of MTT was added to remaining 0.1 mL of cells. The cells were incubated at 37° C. for 2-4 hours, then the O.D was measured at 490-650 nm.

The TNF-α levels released in the culture medium was quantified by ELISA. Tested compounds exhibited inhibitory activity of TNF-α levels (expressed as IC₅₀) Of from about 2.3 nM to about 1000 nM, from about 2.3 nM to about 300 nM, and from about 2.3 nM to about 55 nM.

Tested compounds exhibited p38 kinase inhibitory activity (expressed as IC₅₀) of from about 4.5 nM to about 32000 nM, from about 4.5 nM to about 300 nM and from about 4.5 nM to about 20 nM.

1. A compound having the structure of Formula I: 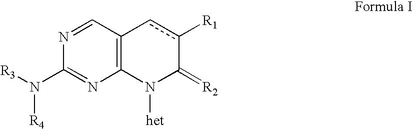
or a pharmaceutically acceptable salt, pharmaceutically acceptable solvate, ester, enantiomer, diastereomer, N-oxide, polymorph or metabolite thereof, wherein
R₁ is alkyl, alkenyl, alkynyl, cycloalkyl, aryl, aralkyl, heteroaryl, heterocyclyl, heteroarylalkyl or heterocyclylalkyl;
R₂ is oxygen, sulphur —NH, —N-acyl, —N(CN), —N(NO₂), —C(R₃)₂ or —CH(NO₂); — [symbol]
represents a single bond or a double bond;
R₃ is hydrogen, alkyl, cycloalkyl, aryl, heterocyclyl, heteroaryl, aralkyl, heteroarylalkyl or heterocyclylalkyl;
R₄ cycloalkyl or heterocyclyl; and
Het is heterocyclyl.

2. A compound selected from:
tert-Butyl 4-{[6-(2-methylphenyl)-7-oxo-8-(tetrahydro-2H-pyran-4-yl)-7,8-dihydropyrido[2,3-d]pyrimidin-2-yl]amino}piperidine-1-carboxylate (Compound No. 1),
Hydrochloride salt of 6-(2-methylphenyl)-2-(piperidin-4-ylamino)-8-(tetrahydro-2H-pyran-4-yl)pyrido[2,3-d]pyrimidin-7(8H)-one (Compound No. 2),
2-[(1-Acetylpiperidin-4-yl)amino]-6-(2-methylphenyl)-8-(tetrahydro-2H-pyran-4-yl)pyrido[2,3-d]pyrimidin-7(8H)-one (Compound No. 3),
6-(2-Methylphenyl)-2-{[1-(methylsulfonyl)piperidin-4-yl]amino}-8-(tetrahydro-2H-pyran-4-yl)pyrido[2,3-d]pyrimidin-7(8H)-one (Compound No. 4),
2-[(1-Benzoylpiperidin-4-yl)amino]-6-(2-methylphenyl)-8-(tetrahydro-2H-pyran-4-yl)pyrido[2,3-d]pyrimidin-7(8H)-one (Compound No. 5),
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 6),
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 7),
2-{[1-(Ethylsulfonyl)piperidin-4-yl]amino}-6-(2-methylphenyl)-8-(tetrahydro-2H-pyran-4-yl)pyrido[2,3-d]pyrimidin-7(8H)-one (Compound No. 8),
2-[(1-Benzyl-piperidin-4-yl)amino]-6-(2-methyl-phenyl)-8-(tetrahydro-2H-pyran-4-yl)pyrido[2,3-d]pyrimidin-7(8H)-one (Compound No. 9),
N-(4-Fluorophenyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 10),
Methylphenyl)-7-oxo-8-(tetrahydro-2H-pyran-4-yl)-7,8-dihydropyrido[2,3-d]pyrimidin-2-yl]amino}-N-[4-(trifluoromethyl)phenyl]piperidine-1-carboxamide (Compound No. 11),
6-(2-Methylphenyl)-2-[(1-methylpiperidin-4-yl)amino]-8-(tetrahydro-2H-pyran-4-yl)pyrido[2,3-d]pyrimidin-7(8H)-one (Compound No. 12),
6-(2-Methylphenyl)-2-[(4-methylpiperazin-1-yl)amino]-8-(tetrahydro-2H-pyran-4-yl)pyrido[2,3-d]pyrimidin-7(8H)-one (Compound No. 13),
2-{[1-(Isopropylsulfonyl)piperidin-4-yl]amino}-6-(2-methylphenyl)-8-(tetrahydro-2H-pyran-4-yl)pyrido[2,3-d]pyrimidin-7(8H)-one (Compound No. 14),
tert-Butyl 4-{[6-(2-chlorophenyl)-7-oxo-8-(tetrahydro-2H-pyran-4-yl)-7,8-dihydropyrido[2,3-d]pyrimidin-2-yl]amino}piperidine-1-carboxylate (Compound No. 15),
6-(2-Methylphenyl)-2-(piperidin-1-ylamino)-8-(tetrahydro-2H-pyran-4-yl)pyrido[2,3-d]pyrimidin-7(8H)-one (Compound No. 16),
2-(Cyclobutylamino)-6-(2-methylphenyl)-8-(tetrahydro-2H-pyran-4-yl)pyrido[2,3-d]pyrimidin-7(8H)-one (Compound No. 17),
4-{[6-(2-Methylphenyl)-7-oxo-8-(tetrahydro-2H-pyran-4-yl)-7,8-dihydropyrido[2,3-d]pyrimidin-2-yl]amino}-N-propylpiperidine-1-carboxamide (Compound No. 18),
N-[(1S)-1,2-Dimethylpropyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide-(Compound No. 19),
N-Cyclohexyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 20),
2-{[1-(4-Fluorobenzoyl)piperidin-4-yl]amino}-6-(2-methylphenyl)-8-(tetrahydro-2H-pyran-4-yl)pyrido[2,3-d]pyrimidin-7(8H)-one (Compound No. 21),
N-(Cyclopentylmethyl)-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 22),
4-{[6-(2-Methylphenyl)-7-oxo-8-(tetrahydro-2H-pyran-4-yl)-7,8-dihydropyrido[2,3-d]pyrimidin-2-yl]amino}-N-(1,1,3,3-tetramethylbutyl)piperidine-1-carboxamide (Compound No. 23),
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carboxamide (Compound No. 24),
N-Cyclopentyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 25),
N-Isopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 26),
4-{[6-(2-Methylphenyl)-7-oxo-8-(tetrahydro-2H-pyran-4-yl)-7,8-dihydropyrido[2,3-d]pyrimidin-2-yl]amino}-N-octylpiperidine-1-carbothioamide (Compound No. 27),
N-tert-Butyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carbothioamide (Compound No. 28),
6-(2-Methylphenyl)-2-[(1-pyrimidin-2-ylpiperidin-4-yl)amino]-8-(tetrahydro-2H-pyran-4-yl)pyrido[2,3-d]pyrimidin-7(8H)-one (Compound No. 29),
N-Cyclopropyl-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 30),
N-[(1R)-1-Cyclohexylethyl]-4-{[6-(2-methylphenyl)-7-oxo-8-(tetrahydro-2H-pyran-4-yl)-7,8-dihydropyrido[2,3-d]pyrimidin-2-yl]amino}piperidine-1-carboxamide (Compound No. 31),
2-{[1-(Cyclopentylcarbonyl)piperidin-4-yl]amino}-6-(2-methylphenyl)-8-(tetrahydro-2H-pyran-4-yl)pyrido[2,3-d]pyrimidin-7(8H)-one (Compound No. 32),
6-(2-Methylphenyl)-2-{[1-(pyrrolidin-1-ylcarbonyl)piperidin-4-yl]amino}-8-(tetrahydro-2H-pyran-4-yl)pyrido[2,3-d]pyrimidin-7(8H)-one (Compound No. 33),
6-(2-Methylphenyl)-8-(tetrahydro-2H-pyran-4-yl)-2-(tetrahydro-2H-pyran-4-ylamino)pyrido[2,3-d]pyrimidin-7(8H)-one (Compound No. 34),
6-(2-Methylphenyl)-2-[(1,2,2,6,6-pentamethylpiperidin-4-yl)amino]-8-(tetrahydro-2H-pyran-4-yl)pyrido[2,3-d]pyrimidin-7(8H)-one (Compound No. 35),
tert-Butyl 4-{[6-(2-methylphenyl)-7-oxo-8-(tetrahydrofuran-3-yl)-7,8-dihydropyrido[2,3-d]pyrimidin-2-yl]amino}piperidine-1-carboxylate (Compound No. 36),
N-Isopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 37),
6-(2-Methylphenyl)-7-oxo-2-{[1-(pyrrolidin-1-ylcarbonyl)piperidin-4-yl]amino}-1-(tetrahydrofuran-3-yl)-7,8-dihydropyrido[2,3-d]pyrimidin-1-ium (Compound No. 38),
2-{[1-(Ethylsulfonyl)piperidin-4-yl]amino}-6-(2-methylphenyl)-7-oxo-1-(tetrahydrofuran-3-yl)-7,8-dihydropyrido[2,3-d]pyrimidin-1-ium (Compound No. 39),
6-(2-Methylphenyl)-2-{[1-(methylsulfonyl)piperidin-4-yl]amino}-8-(tetrahydrofuran-3-yl)pyrido[2,3-d]pyrimidin-7(8H)-one (Compound No. 40),
N-Cyclopropyl-4-{[6-(2-methylphenyl)-7-oxo-8-(tetrahydrofuran-3-yl)-7,8-dihydropyrido[2,3-d]pyrimidin-2-yl]amino}piperidine-1-carboxamide (Compound No. 41),
6-(2-Methylphenyl)-2-{[(3S)-1-(methylsulfonyl)pyrrolidin-3-yl]amino}-8-(tetrahydrofuran-3-yl)pyrido[2,3-d]pyrimidin-7(8H)-one (Compound No. 42),
tert-Butyl 4-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate (Compound No. 43),
6-(2-Methylphenyl)-2-{[1-(methylsulfonyl)piperidin-4-yl]amino}-8-[(3S)-tetrahydrofuran-3-yl]pyrido[2,3-d]pyrimidin-7(8H)-one (Compound No. 44),
N-Isopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 45),
6-(2-Methylphenyl)-2-{[1-(methylsulfonyl)piperidin-4-yl]amino}-8-[(3R)-tetrahydrofuran-3-yl]pyrido[2,3-d]pyrimidin-7(8H)-one (Compound No. 46),
N-Isopropyl-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 47),
tert-Butyl 4-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 48),
Hydrochloride salt of 6-(2-methylphenyl)-2-(piperidin-4-ylamino)-8-[(3R)-tetrahydrofuran-3-yl]pyrido[2,3-d]pyrimidin-7(8H)-one (Compound No. 49),
Hydrochloride salt 6-(2-methylphenyl)-2-(piperidin-4-ylamino)-8-[(3S)-tetrahydrofuran-3-yl]pyrido[2,3-d]pyrimidin-7(8H)-one (Compound No. 50),
2-{([1-(Ethylsulfonyl)piperidin-4-yl]amino}-6-(2-methylphenyl)-8-[(3S)-tetrahydrofuran-3-yl]pyrido[2,3-d]pyrimidin-7(8H)-one (Compound No. 51),
N-Cyclopropyl-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 52),
N-Cyclopropyl-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 53),
2-[(1-Benzylpiperidin-4-yl)amino]-6-(2-methylphenyl)-8-[(3R)-tetrahydrofuran-3-yl]pyrido[2,3-d]pyrimidin-7(8H)-one (Compound No. 54),
2-(Cyclobutylamino)-6-(2-methylphenyl)-8-[(3R)-tetrahydrofuran-3-yl]pyrido[2,3-d]pyrimidin-7(8H)-one (Compound No. 55),
2-(Cyclopropylamino)-6-(2-methylphenyl)-8-[(3R)-tetrahydrofuran-3-yl]pyrido[2,3-d]pyrimidin-7(8H)-one (Compound No. 56),
2-{[1-(Ethylsulfonyl)piperidin-4-yl]amino}-6-(2-methylphenyl)-8-[(3R)-tetrahydrofuran-3-yl]pyrido[2,3-d]pyrimidin-7(8H)-one (Compound No. 57),
2-[(1-Acetylpiperidin-4-yl)amino]-6-(2-methylphenyl)-8-[(3R)-tetrahydrofuran-3-yl]pyrido[2,3-d]pyrimidin-7(8H)-one (Compound No. 58),
2-{[1-(4-Fluorobenzoyl)piperidin-4-yl]amino}-6-(2-methylphenyl)-8-[(3R)-tetrahydrofuran-3-yl]pyrido[2,3-d]pyrimidin-7(8H)-one (Compound No. 59),
2-{[1-(Cyclopropylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 60),
2-{[1-(Cyclopropylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 61),
2-{[1-(Cyclopentylcarbonyl)piperidin-4-yl]amino}-6-(2-methylphenyl)-8-[(3S)-tetrahydrofuran-3-yl]pyrido[2,3-d]pyrimidin-7(8H)-one (Compound No. 62),
2-{[1-(Cyclopentylcarbonyl)piperidin-4-yl]amino}-6-(2-methylphenyl)-8-[(3R)-tetrahydrofuran-3-yl]pyrido[2,3-d]pyrimidin-7(8H)-one (Compound No. 63),
2-[(1-Benzoylpiperidin-4-yl)amino]-6-(2-methylphenyl)-8-[(3S)-tetrahydrofuran-3-yl]pyrido[2,3-d]pyrimidin-7(8H)-one (Compound No. 64),
2-[(1-Benzoylpiperidin-4-yl)amino]-6-(2-methylphenyl)-8-[(3R)-tetrahydrofuran-3-yl]pyrido[2,3-d]pyrimidin-7(8H)-one (Compound No. 65),
2-{[1-(4-Fluorobenzoyl)piperidin-4-yl]amino}-6-(2-methylphenyl)-8-[(3S)-tetrahydrofuran-3-yl]pyrido[2,3-d]pyrimidin-7(8H)-one (Compound No. 66),
N-(4-Fluorophenyl)-4-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 67),
2-[(1-Acetylpiperidin-4-yl)amino]-6-(2-methylphenyl)-8-[(3S)-tetrahydrofuran-3-yl]pyrido[2,3-d]pyrimidin-7(8H)-one (Compound No. 68),
N-(4-Fluorophenyl)-4-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 69),
tert-Butyl (3S)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 70),
(3S)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 71)
tert-Butyl (3S)-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxylate (Compound No. 72),
tert-Butyl (3R)-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxylate (Compound No. 73),
(3R)—N-Isopropyl-3-({6-(2-methylphenyl)-7-oxo-8-[(3R)-tetrahydrofuran-3-yl]-7,8-dihydropyrido[2,3-d]pyrimidin-2-yl}amino)piperidine-1-carboxamide (Compound No. 74),
(3S)—N-Isopropyl-3-({6-(2-methylphenyl)-7-oxo-8-[(3S)-tetrahydrofuran-3-yl]-7,8-dihydropyrido[2,3-d]pyrimidin-2-yl}amino)piperidine-1-carboxamide (Compound No. 75),
6-(2-Methylphenyl)-2-{[(3S)-1-(methylsulfonyl)piperidin-3-yl]amino}-8-[(3S)-tetrahydrofuran-3-yl]pyrido[2,3-d]pyrimidin-7(8H)-one (Compound No. 76),
6-(2-Methylphenyl)-2-{[1-(methylsulfonyl)piperidin-4-yl]amino}-8-[(3S)-1-(methylsulfonyl)pyrrolidin-3-yl]pyrido[2,3-d]pyrimidin-7(8H)-one (Compound No. 77),
6-(2-Methylphenyl)-2-{[1-(methylsulfonylpiperidin-4-yl]amino}-8-[(3S)-1-(methylsulfonyl)pyrrolidin-3-yl]pyrido[2,3-d]pyrimidin-7(8H)-one (Compound No. 78),
tert-Butyl 4-({6-(2-methylphenyl)-7-oxo-8-[(3S)-pyrrolidin-3-yl]-7,8-dihydropyrido[2,3-d]pyrimidin-2-yl}amino)piperidine-1-carboxylate (Compound No. 79)
4-({8-[(3S)-1-Acetylpyrrolidin-3-yl]-6-(2-methylphenyl)-7-oxo-7,8-dihydropyrido[2,3-d]pyrimidin-2-yl}amino)-N-isopropylpiperidine-1-carboxamide (Compound No. 80),
Hydrochloride salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 81),
tert-Butyl (3S)-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxylate (Compound No. 82),
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 83),
(3S)—N-Isopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carbothioamide (Compound No. 84),
6-(2-Methylphenyl)-2-{[(3S)-1-(methylsulfonyl)pyrrolidin-3-yl]amino}-8-(tetrahydro-2H-pyran-4-yl)pyrido[2,3-d]pyrimidin-7(8H)-one (Compound No. 85),
2-{[(3S)-1-(Ethylsulfonyl)pyrrolidin-3-yl]amino}-6-(2-methylphenyl)-8-(tetrahydro-2H-pyran-4-yl)pyrido[2,3-d]pyrimidin-7(8H)-one (Compound No. 86),
2-{[(3S)-1-Acetylpyrrolidin-3-yl]amino}-6-(2-methylphenyl)-8-(tetrahydro-2H-pyran-4-yl)pyrido[2,3-d]pyrimidin-7(8H)-one (Compound No. 87),
(3S)—N-Cyclopropyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 88),
(3S)—N-Butyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 90),
(3S)—N-Cyclopentyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 91),
(3S)—N-[(1S)-1,2-Dimethylpropyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 92),
2-{[(3R)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 93),
2-{[(3S)-1-Benzylpyrrolidin-3-yl]amino}-6-(2-methylphenyl)-8-(tetrahydro-2H-pyran-4-yl)pyrido[2,3-d]pyrimidin-7(8H)-one (Compound No. 94),
(3S)—N-Cyclohexyl-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 95),
(3S)-3-{[6-(2-Methylphenyl)-7-oxo-8-(tetrahydro-2H-pyran-4-yl)-7,8-dihydropyrido[2,3-d]pyrimidin-2-yl]amino}-N-octylpyrrolidine-1-carboxamide (Compound No. 96),
2-{[(3S)-1-(Cyclopropylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 97),
2-{[(3S)-1-(Cyclopentylcarbonyl)pyrrolidin-3-yl]amino}-6-(2-methylphenyl)-8-(tetrahydro-2H-pyran-4-yl)pyrido[2,3-d]pyrimidin-7(8H)-one (Compound No. 98),
6-(2-Methylphenyl)-2-{[(3S)-1-pyrimidin-2-yl-pyrrolidin-3-yl]amino}-8-(tetrahydro-2H-pyran-4-yl)pyrido[2,3-d]pyrimidin-7(8H)-one (Compound No. 99)
(3S)—N-[(1R)-1-Cyclohexylethyl]-3-{[6-(2-methylphenyl)-7-oxo-8-(tetrahydro-2H-pyran-4-yl)-7,8-dihydropyrido[2,3-d]pyrimidin-2-yl]amino}pyrrolidine-1-carboxamide (Compound No. 100),
6-(2-Methylphenyl)-2-{[(3S)-1-(pyrrolidin-1-ylcarbonyl)pyrrolidin-3-yl]amino}-8-(tetrahydro-2H-pyran-4-yl)pyrido[2,3-d]pyrimidin-7(8H)-one (Compound No. 101),
2-{[(3S)-1-(Cyclopentylacetyl)pyrrolidin-3-yl]amino}-6-(2-methylphenyl)-8-(tetrahydro-2H-pyran-4-yl)pyrido[2,3-d]pyrimidin-7(8H)-one (Compound No. 102),
tert-Butyl (3S)-3-{[6-(2-methylphenyl)-7-oxo-8-(tetrahydrofuran-3-yl)-7,8-dihydropyrido[2,3-d]pyrimidin-2-yl]amino}pyrrolidine-1-carboxylate (Compound No. 103),
tert-Butyl 4-({8-[(3S)-1-benzylpyrrolidin-3-yl]-6-(2-methylphenyl)-7-oxo-7,8-dihydropyrido[2,3-d]pyrimidin-2-yl}amino)piperidine-1-carboxylate (Compound No. 104),
(3S)—N-Isopropyl-3-{[6-(2-methylphenyl)-7-oxo-8-(tetrahydrofuran-3-yl)-7,8-dihydropyrido[2,3-d]pyrimidin-2-yl]amino}pyrrolidine-1-carboxamide (Compound No. 105),
tert-Butyl 4-{[8-(1-benzylpiperidin-4-yl)-6-(2-methylphenyl)-7-oxo-7,8-dihydropyrido[2,3-d]pyrimidin-2-yl]amino}piperidine-1-carboxylate (Compound No. 106),
tert -Butyl 4-{[6-(2-methylphenyl)-8-(1-methylpiperidin-4-yl)-7-oxo-7,8-dihydropyrido[2,3-d]pyrimidin-2-yl]amino}piperidine-1-carboxylate (Compound No. 107)
8-(1-Benzylpiperidin-4-yl)-6-(2-methylphenyl)-2-[(1-methylpiperidin-4-yl)amino]pyrido[2,3-d]pyrimidin-7(8H)-one (Compound No. 108),
8-(1-Benzylpiperidin-4-yl)-2-(cyclobutylamino)-6-(2-methylphenyl)pyrido[2,3-d]pyrimidin-7(8H)-one (Compound No. 109),
tert-Butyl (3S)-3-{[8-(1-benzylpiperidin-4-yl)-6-(2-methylphenyl)-7-oxo-7,8-dihydropyrido[2,3-d]pyrimidin-2-yl]amino}pyrrolidine-1-carboxylate (Compound No. 110),
(3S)-3-{[8-(1-Benzylpiperidin-4-yl)-6-(2-methylphenyl)-7-oxo-7,8-dihydropyrido[2,3-d]pyrimidin-2-yl]amino}-N-isopropylpyrrolidine-1-carboxamide (Compound No. 111),
Hydrochloride salt of S-(1-benzylpiperidin-4-yl)-6-(2-methylphenyl)-2-(piperidin-4-ylamino)pyrido[2,3-d]pyrimidin-7(8H)-one (Compound No. 112),
Hydrochloride salt of 6-(2-methylphenyl)-8-(1-methylpiperidin-4-yl)-2-(piperidin-4-ylamino)pyrido[2,3-d]pyrimidin-7(8H)-one (Compound No. 113),
4-{[8-(1-Benzylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 114),
4-{[8-(1-Benzylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 115),
4-{[8-(1-Benzylpiperidin-4-yl)-6-(2-methylphenyl)-7-oxo-7,8-dihydropyrido[2,3-d]pyrimidin-2-yl]amino}-N-cyclopentylpiperidine-1-carboxamide (Compound No. 116),
4-{[8-(1-Benzylpiperidin-4-yl)-6-(2-methylphenyl)-7-oxo-7,8-dihydropyrido[2,3-d]pyrimidin-2-yl]amino}-N-isobutylpiperidine-1-carboxamide (Compound No. 117),
4-{[8-(1-Benzylpiperidin-4-yl)-6-(2-methylphenyl)-7-oxo-7,8-dihydropyrido[2,3-d]pyrimidin-2-yl]amino}-N-[(1R)-1-cyclohexylethyl]piperidine-1-carboxamide (Compound No. 118),
N-Isopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 119),
N-Cyclopropyl-4-{[6-(2-methylphenyl)-8-(1-methylpiperidin-4-yl)-7-oxo-7,8-dihydropyrido[2,3-d]pyrimidin-2-yl]amino}piperidine-1-carboxamide (Compound No. 120),
8-(1-Benzylpiperidin-4-yl)-2-{[1-(cyclopentylacetyl)piperidin-4-yl]amino}-6-(2-methylphenyl)pyrido[2,3-d]pyrimidin-7(8H)-one (Compound No. 121),
8-(1-Benzylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 122),
8-(1-Benzylpiperidin-4-yl)-6-(2-methylphenyl)-2-{[1-(phenylsulfonyl)piperidin-4-yl]amino}pyrido[2,3-d]pyrimidin-7(8H)-one (Compound No. 123),
8-(1-Benzylpiperidin-4-yl)-6-(2-methylphenyl)-2-{[1-(methylsulfonyl)piperidin-4-yl]amino}pyrido[2,3-d]pyrimidin-7(8H)-one (Compound No. 124),
8-(1-Benzylpiperidin-4-yl)-2-{[1-(ethylsulfonyl)piperidin-4-yl]amino}-6-(2-methylphenyl)pyrido[2,3-d]pyrimidin-7(8H)-one (Compound no. 125),
6-(2-Methylphenyl)-8-(1-methylpiperidin-4-yl)-2-{[1-(methylsulfonyl)piperidin-4-yl]amino}pyrido[2,3-d]pyrimidin-7(8H)-one (Compound No. 126),
2-{[1-(Ethylsulfonyl)piperidin-4-yl]amino}-6-(2-methylphenyl)-8-(1-methylpiperidin-4-yl)pyrido[2,3-d]pyrimidin-7(8H)-one (Compound No. 127),
2-{[1-(Isopropylsulfonyl)piperidin-4-yl]amino}-6-(2-methylphenyl)-8-(1-methylpiperidin-4-yl)pyrido[2,3-d]pyrimidin-7(8H)-one (Compound No. 128),
6-(2-Methylphenyl)-8-(1-methylpiperidin-4-yl)-2-{[1-(pyrrolidin-1-ylcarbonyl)piperidin-4-yl]amino}pyrido[2,3-d]pyrimidin-7(8H)-one (Compound No. 129),
2-[(1-Benzoylpiperidin-4-yl)amino]-6-(2-methylphenyl)-8-(1-methylpiperidin-4-yl)pyrido[2,3-d]pyrimidin-7(8H)-one (Compound No. 130),
2-[(1-Benzoylpiperidin-4-ylamino]-6-(2-methylphenyl)-8-(1-methylpiperidin-4-yl)pyrido[2,3-d]pyrimidin-7(8H)-one (Compound No. 130),
tert-Butyl 4-{[6-(2-methylphenyl)-7-oxo-8-piperidin-4-yl-7,8-dihydropyrido[2,3-d]pyrimidin-2-yl]amino}piperidine-1-carboxylate (Compound No. 131),
6-(2-Methylphenyl)-8-piperidin-4-yl-2-(piperidin-4-ylamino)pyrido[2,3-d]pyrimidin-7(8H)-one (Compound No. 132),
N-Isopropyl-4-{[6-(2-methylphenyl)-7-oxo-8-piperidin-4-yl-7,8-dihydropyrido[2,3-d]pyrimidin-2-yl]amino}piperidine-1-carboxamide (Compound No. 133),
tert-Butyl 4-{[8-{1-[(isopropylamino)carbonyl]piperidin-4-yl}-6-(2-methylphenyl)-7-oxo-7,8-dihydropyrido[2,3-d]pyrimidin-2-yl]amino}piperidine-1-carboxylate (Compound No. 134),
Hydrochloride salt of N-isopropyl-4-[6-(2-methylphenyl)-7-oxo-2-(piperidin-4-ylamino)pyrido[2,3-d]pyrimidin-8(7H)-yl]piperidine-1-carboxamide (Compound No. 135),
N-Isopropyl-4-[2-({1-[(isopropylamino)carbonyl]piperidin-4-yl}amino)-6-(2-methylphenyl)-7-oxopyrido[2,3-d]pyrimidin-8(7H)-yl]piperidine-1-carboxamide (Compound No. 136),
4-[2-({1-[(Cyclopropylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 137),
4-[2-({1-[(tert-Butylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 138),
4-[2-({1-[(Cyclohexylamino)carbonyl]piperidin-4-yl}amino)-6-(2-methylphenyl)-7-oxopyrido[2,3-d]pyrimidin-8(7H)-yl]-N-isopropylpiperidine-1-carboxamide (Compound No. 139),
N-Isopropyl-4-[6-(2-methylphenyl)-2-{[1-(morpholin-4-ylcarbonyl)piperidin-4-yl]amino}-7-oxopyrido[2,3-d]pyrimidin-8(7H)-yl]piperidine-1-carboxamide (Compound No. 140),
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 141),
N-Isopropyl-4-[6-(2-methylphenyl)-2-{[1-(methylsulfonyl)piperidin-4-yl]amino}-7-oxopyrido[2,3-d]pyrimidin-8(7H)-yl]piperidine-1-carboxamide (Compound No. 142),
4-[2-{[1-(Ethylsulfonyl)piperidin-4-yl]amino}-6-(2-methylphenyl)-7-oxopyrido[2,3-d]pyrimidin-8(7H)-yl]-N-isopropylpiperidine-1-carboxamide (Compound No. 143),
N-Isopropyl-4-[2-{[1-(isopropylsulfonyl)piperidin-4-yl]amino}-6-(2-methylphenyl)-7-oxopyrido[2,3-d]pyrimidin-8(7H)-yl]piperidine-1-carboxamide (Compound No. 144),
4-[2-{[1-(Cyclopropylcarbonyl)piperidin-4-yl]amino}-6-(2-methylphenyl)-7-oxopyrido[2,3-d]pyrimidin-8(7H)-yl]-N-isopropylpiperidine-1-carboxamide (Compound No. 145),
4-[2-[(1-Benzoylpiperidin-4-yl)amino]-6-(2-methylphenyl)-7-oxopyrido[2,3-d]pyrimidin-8(7H)-yl]-N-isopropylpiperidine-1-carboxamide (Compound No. 146),
4-[2-[(1-Acetylpiperidin-4-yl)amino]-6-(2-methylphenyl)-7-oxopyrido[2,3-d]pyrimidin-8(7H)-yl]-N-isopropylpiperidine-1-carboxamide (Compound No. 147),
N-Isopropyl-4-[6-(2-methylphenyl)-2-[(1-methylpiperidin-4-yl)amino]-7-oxopyrido[2,3-d]pyrimidin-8(7H)-yl]piperidine-1-carboxamide (Compound No. 148),
4-[2-[(1-Benzylpiperidin-4-yl)amino]-6-(2-methylphenyl)-7-oxopyrido[2,3-d]pyrimidin-8(7H)-yl]-N-isopropylpiperidine-1-carboxamide (Compound No. 149),
8-(1-Acetylpiperidin-4-yl)-2-[(1-acetylpiperidin-4-yl)amino]-6-(2-methylphenyl)pyrido[2,3-d]pyrimidin-7(8H)-one (Compound No. 150),
4-[2-{[1-(4-Fluorobenzoyl)piperidin-4-yl]amino}-6-(2-methylphenyl)-7-oxopyrido[2,3-d]pyrimidin-8(7H)-yl]-N-isopropylpiperidine-1-carboxamide (Compound No. 151),
tert-Butyl 4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxylate (Compound No. 152),
Hydrochloride salt of 6-(2-methylphenyl)-8-[1-(methylsulfonylpiperidin-4-yl]-2-(piperidin-4-ylamino)pyrido[2,3-d]pyrimidin-7(8H)-one (Compound No. 153),
N-Isopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 154),
N-(tert-Butyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 155),
N-Cyclohexyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 156),
N-(4-Fluorophenyl)-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 157),
6-(2-Methylphenyl)-8-[1-(methylsulfonyl)piperidin-4-yl]-2-{[1-(morpholin-4-ylcarbonyl)piperidin-4-yl]amino}pyrido[2,3-d]pyrimidin-7(8H)-one (Compound No. 158),
6-(2-Methylphenyl)-8-[1-(methylsulfonyl)piperidin-4-yl]-2-{[1-(methylsulfonyl)piperidin-4-yl]amino}pyrido[2,3-d]pyrimidin-7(8H)-one (Compound No. 159),
2-{[1-(Ethylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 160),
2-{[1-(Isopropylsulfonyl)piperidin-4-yl]amino}-6-(2-methylphenyl)-8-[1-(methylsulfonyl)piperidin-4-yl]pyrido[2,3-d]pyrimidin-7(8H)-one (Compound No. 161),
2-{[1-(Cyclopropylcarbonyl)piperidin-4-yl]amino}-6-(2-methylphenyl)-8-[1-(methylsulfonyl)piperidin-4-yl]pyrido[2,3-d]pyrimidin-7(8H)-one (Compound 162),
2-[(1-Acetylpiperidin-4-yl)amino]-6-(2-methylphenyl)-8-[1-(methylsulfonyl)piperidin-4-yl]pyrido[2,3-d]pyrimidin-7(8H)-one (Compound No. 163),
2-[(1-Benzoylpiperidin-4-yl)amino]-6-(2-methylphenyl)-8-[1-(methylsulfonyl)piperidin-4-yl]pyrido[2,3-d]pyrimidin-7(8H)-one (Compound No. 164),
2-{[1-(4-Fluorobenzoyl)piperidin-4-yl]amino}-6-(2-methylphenyl)-8-[1-(methylsulfonyl)piperidin-4-yl]pyrido[2,3-d]pyrimidin-7(8H)-one (Compound No. 165),
tert-Butyl 4-{[8-(1-acetylpiperidin-4-yl)-6-(2-methylphenyl)-7-oxo-7,8-dihydropyrido[2,3-d]pyrimidin-2-yl]amino}piperidine-1-carboxylate (Compound No. 166),
8-(1-Acetylpiperidin-4-yl)-6-(2-methylphenyl)-2-(piperidin-4-ylamino)pyrido[2,3-d]pyrimidin-7(8H)-one (Compound No. 167),
4-{[8-(1-Acetylpiperidin-4-yl)-6-(2-methylphenyl)-7-oxo-7,8-dihydropyrido[2,3-d]pyrimidin-2-yl]amino}-N-isopropylpiperidine-1-carboxamide (Compound No. 168),
4-{[8-(1-Acetylpiperidin-4-yl)-6-(2-methylphenyl)-7-oxo-7,8-dihydropyrido[2,3-d]pyrimidin-2-yl]amino}-N-cyclopropylpiperidine-1-carboxamide (Compound No. 169),
4-{[8-(1-Acetylpiperidin-4-yl)-6-(2-methylphenyl)-7-oxo-7,8-dihydropyrido[2,3-d]pyrimidin-2-yl]amino}-N-(tert-butylpiperidine-1-carboxamide (Compound No. 170),
4-{[8-(1-Acetylpiperidin-4-yl)-6-(2-methylphenyl)-7-oxo-7,8-dihydropyrido[2,3-d]pyrimidin-2-yl]amino}-N-cyclohexylpiperidine-1-carboxamide (Compound No. 171),
4-{[8-(1-Acetylpiperidin-4-yl)-6-(2-methylphenyl)-7-oxo-7,8-dihydropyrido[2,3-d]pyrimidin-2-yl]amino}-N-(4-fluorophenyl)piperidine-1-carboxamide (Compound No. 172),
8-(1-Acetylpiperidin-4-yl)-6-(2-methylphenyl)-2-{[1-(morpholin-4-ylcarbonyl)piperidin-4-yl]amino}pyrido[2,3-d]pyrimidin-7(8H)-one (Compound No. 173),
8-(1-Acetylpiperidin-4-yl)-6-(2-methylphenyl)-2-{[1-(methylsulfonyl)piperidin-4-yl]amino}pyrido[2,3-d]pyrimidin-7(8H)-one (Compound No. 174),
8-(1-Acetylpiperidin-4-yl)-2-{[1-(ethylsulfonyl)piperidin-4-yl]amino}-6-(2-methylphenyl)pyrido[2,3-d]pyrimidin-7(8H)-one (Compound No. 175),
8-(1-Acetylpiperidin-4-yl)-2-{[1-(isopropylsulfonyl)piperidin-4-yl]amino}-6-(2-methylphenyl)pyrido[2,3-d]pyrimidin-7(8H)-one (Compound No. 176),
8-(1-Acetylpiperidin-4-yl)-2-{[1-(cyclopropylcarbonyl)piperidin-4-yl]amino}-6-(2-methylphenyl)pyrido[2,3-d]pyrimidin-7(8H)-one (Compound No. 177),
8-(1-Acetylpiperidin-4-yl)-2-{[1-(4-fluorobenzoyl)piperidin-4-yl]amino}-6-(2-methylphenyl)pyrido[2,3-d]pyrimidin-7(8H)-one (Compound No. 178),
tert-Butyl 4-{[8-[(3S)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 179),
Hydrochloride salt of 8-[(3S)-1-acetylpyrrolidin-3-yl]-6-(2-methylphenyl)-2-(piperidin-4-ylamino)pyrido[2,3-d]pyrimidin-7(8H)-one (Compound No. 180),
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 181),
2-[(1-Acetylpiperidin-4-yl)amino]-6-(2-methylphenyl)-8-(1-methylpiperidin-4-yl)pyrido[2,3-d]pyrimidin-7(8H)-one (Compound No. 182),
Hydrochloride salt of 6-(2-methylphenyl)-8-[(3S)-1-(methylsulfonyl)pyrrolidin-3-yl]-2-(piperidin-4-ylamino)pyrido[2,3-d]pyrimidin-7(8H)-one (Compound No. 183),
tert-Butyl 4-{[8-[(3R)-1-acetylpyrrolidin-3-yl]-6-(2-methylphenyl)-7-oxo-7,8-dihydropyrido[2,3-d]pyrimidin-2-yl]amino}piperidine-1-carboxylate (Compound No. 184),
Hydrochloride salt of 6-(2-methylphenyl)-2-[(3S)-pyrrolidin-3-ylamino]-8-(tetrahydrofuran-3-yl)pyrido[2,3-d]pyrimidin-7(8H)-one (Compound No. 185),
Hydrochloride salt of 6-(2-methylphenyl)-2-[(3S)-piperidin-3-ylamino]-8-[(3R)-tetrahydrofuran-3-yl]pyrido[2,3-d]pyrimidin-7(8H)-one (Compound No. 186),
Hydrochloride salt of 6-(2-methylphenyl)-2-[(3S)-piperidin-3-ylamino]-8-[(3S)-tetrahydrofuran-3-yl]pyrido[2,3-d]pyrimidin-7(8H)-one (Compound No. 187),
Hydrochloride salt of 6-(2-methylphenyl)-2-[(3R)-piperidin-3-ylamino]-8-[(3R)-tetrahydrofuran-3-yl]pyrido[2,3-d]pyrimidin-7(8H)-one (Compound No. 188),
4-[2-[(1-{[(4-Fluorophenyl)amino]carbonyl}piperidin-4-yl)amino]-6-(2-methylphenyl)-7-oxopyrido[2,3-d]pyrimidin-8(7H)-yl]-N-isopropylpiperidine-1-carboxamide (Compound No. 189),
4-{[8-{1-[(Isopropylamino)carbonyl]piperidin-4-yl}-6-(2-methylphenyl)-7-oxo-7,8-dihydropyrido[2,3-d]pyrimidin-2-yl]amino}-N-morpholin-4-ylpiperidine-1-carboxamide (Compound No. 190),
4-[2-{[1-(2,2-Dimethylpropanoyl)piperidin-4-yl]amino}-6-(2-methylphenyl)-7-oxopyrido[2,3-d]pyrimidin-8(7H)-yl]-N-isopropylpiperidine-1-carboxamide (Compound No. 191),
8-[(3S)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 192),
N-Cyclopropyl-4-({6-(2-methylphenyl)-8-[1-(methylsulfonyl)piperidin-4-yl]-7-oxo-7,8-dihydropyrido[2,3-d]pyrimidin-2-yl}amino)piperidine-1-carboxamide (Compound No. 193),
4-({6-(2-Methylphenyl)-8-[1-(methylsulfonyl)piperidin-4-yl]-7-oxo-7,8-dihydropyrido[2,3-d]pyrimidin-2-yl}amino)-N-morpholin-4-ylpiperidine-1-carboxamide (Compound No. 194),
2-{[1-(2,2-Dimethylpropanoyl)piperidin-4-yl]amino}-6-(2-methylphenyl)-8-[1-(methylsulfonyl)piperidin-4-yl]pyrido[2,3-d]pyrimidin-7(8H)-one (Compound No. 195),
6-(2-Methylphenyl)-2-[(1-methylpiperidin-4-yl)amino]-8-[1-(methylsulfonyl)piperidin-4-yl]pyrido[2,3-d]pyrimidin-7(8H)-one (Compound No. 196),
2-[(1-Benzylpiperidin-4-yl)amino]-6-(2-methylphenyl)-8-[1-(methylsulfonyl)piperidin-4-yl]pyrido[2,3-d]pyrimidin-7(8H)-one (Compound No. 197),
8-(1-Acetylpiperidin-4-yl)-2-[(1-benzoylpiperidin-4-yl)amino]-6-(2-methylphenyl)pyrido[2,3-d]pyrimidin-7(8H)-one (Compound No. 198),
8-(1-Acetylpiperidin-4-yl)-2-{[1-(2,2-dimethylpropanoyl)piperidin-4-yl]amino}-6-(2-methylphenyl)pyrido[2,3-d]pyrimidin-7(8H)-one (Compound No. 199),
8-(1-Acetylpiperidin-4-yl)-6-(2-methylphenyl)-2-[(1-methylpiperidin-4-yl)amino]pyrido[2,3-d]pyrimidin-7(8H)-one (Compound No. 200),
8-(1-Acetylpiperidin-4-yl)-2-[(1-benzylpiperidin-4-yl)amino]-6-(2-methylphenyl)pyrido[2,3-d]pyrimidin-7(8H)-one (Compound No. 201),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 202),
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 203),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 204),
4-{[8-[(3S)-1-Acetylpyrrolidin-3-yl]-6-(2-methylphenyl)-7-oxo-7,8-dihydropyrido[2,3-d]pyrimidin-2-yl]amino}-N-(4-fluorophenyl)piperidine-1-carboxamide (Compound No. 205),
4-{[8-[(3S)-1-Acetylpyrrolidin-3-yl]-6-(2-methylphenyl)-7-oxo-7,8-dihydropyrido[2,3-d]pyrimidin-2-yl]amino}-N-morpholin-4-ylpiperidine-1-carboxamide (Compound No. 206),
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 207),
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one ((Compound No. 208),
8-[(3R)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 209),
2-[(1-Acetylpiperidin-4-yl)amino]-8-[(3R)-1-acetylpyrrolidin-3-yl]-6-(2-methylphenyl)pyrido[2,3-d]pyrimidin-7(8H)-one (Compound No. 210),
8-[(3R)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 211),
8-[(3R)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 212),
8-[(3R)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 213),
8-[(3R)-1-Acetylpyrrolidin-3-yl]-6-(2-methylphenyl)-2-[(1-methylpiperidin-4-yl)amino]pyrido[2,3-d]pyrimidin-7(8H)-one (Compound No. 214),
8-[(3R)-1-Acetylpyrrolidin-3-yl]-2-[(1-benzylpiperidin-4-yl)amino]-6-(2-methylphenyl)pyrido[2,3-d]pyrimidin-7(8H)-one (Compound No. 215),
8-[(3S)-1-Acetylpyrrolidin-3-yl]-2-{[1-(2,2-dimethylpropanoyl)piperidin-4-yl]amino}-6-(2-methylphenyl)pyrido[2,3-d]pyrimidin-7(8H)-one (Compound No. 216),
8-[(3S)-1-Acetylpyrrolidin-3-yl]-6-(2-methylphenyl)-2-[(1-methylpiperidin-4-yl)amino]pyrido[2,3-d]pyrimidin-7(8H)-one (Compound No. 217),
4-{[8-[(3S)-1-Acetylpyrrolidin-3-yl]-6-(2-methylphenyl)-7-oxo-7,8-dihydropyrido[2,3-d]pyrimidin-2-yl]amino}-N-isopropylpiperidine-1-carboxamide (Compound No. 218),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclopropylpiperidine-1-carboxamide (Compound No. 219),
4-{[8-[(3S)-1-Acetylpyrrolidin-3-yl]-6-(2-methylphenyl)-7-oxo-7,8-dihydropyrido[2,3-d]pyrimidin-2-yl]amino}-N-(tert-butyl)piperidine-1-carboxamide (Compound No. 220),
4-{[8-[(3S)-1-Acetylpyrrolidin-3-yl]-6-(2-methylphenyl)-7-oxo-7,8-dihydropyrido[2,3-d]pyrimidin-2-yl]amino}-N-cyclohexylpiperidine-1-carboxamide (Compound No. 221)
4-{[8-[(3S)-1-Acetylpyrrolidin-3-yl]-6-(2-methylphenyl)-7-oxo-7,8-dihydropyrido[2,3-d]pyrimidin-2-yl]amino}-N-(4-fluorophenyl)piperidine-1-carboxamide (Compound No. 222),
4-{[8-[(3S)-1-Acetylpyrrolidin-3-yl]-6-(2-methylphenyl)-7-oxo-7,8-dihydropyrido[2,3-d]pyrimidin-2-yl]amino}-N-morpholin-4-ylpiperidine-1-carboxamide (Compound No. 223),
8-[(3S)-1-Acetylpyrrolidin-3-yl]-6-(2-methylphenyl)-2-{[1-(methylsulfonyl)piperidin-4-yl]amino}pyrido[2,3-d]pyrimidin-7(8H)-one (Compound No. 224),
8-[(3S)-1-Acetylpyrrolidin-3-yl]-2-{[1-(ethylsulfonyl)piperidin-4-yl]amino}-6-(2-methylphenyl)pyrido[2,3-d]pyrimidin-7(8H)-one (Compound No. 225),
8-[(3S)-1-Acetylpyrrolidin-3-yl]-6-(2-methylphenyl)-2-{[1-(propylsulfonyl)piperidin-4-yl]amino}pyrido[2,3-d]pyrimidin-7(8H)-one (Compound No. 226),
8-[(3S)-1-Acetylpyrrolidin-3-yl]-2-{[1-(cyclopropylcarbonyl)piperidin-4-yl]amino}-6-(2-methylphenyl)pyrido[2,3-d]pyrimidin-7(8H)-one (Compound No. 227),
2-[(1-Acetylpiperidin-4-yl)amino]-8-[(3S)-1-acetylpyrrolidin-3-yl]-6-(2-methylphenyl)pyrido[2,3-d]pyrimidin-7(8H)-one (Compound No. 228),
8-[(3S)-1-Acetylpyrrolidin-3-yl]-2-[(1-benzoylpiperidin-4-yl)amino]-6-(2-methylphenyl)pyrido[2,3-d]pyrimidin-7(8H)-one (Compound No. 229),
8-[(3S)-1-Acetylpyrrolidin-3-yl]-2-{[1-(4-fluorobenzoyl)piperidin-4-yl]amino}-6-(2-methylphenyl)pyrido[2,3-d]pyrimidin-7(8H)-one (Compound No. 230).

3. A method of treating an autoimmune disease, inflammation or associated pathology comprising administering to a mammal in need thereof a therapeutically effective amount of one or more compounds of Formula I or a pharmaceutically acceptable salt thereof, wherein Formula I is represented by: 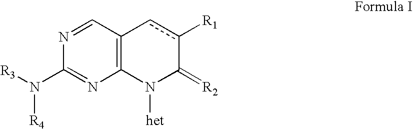
wherein
R₁ is alkyl, alkenyl, alkynyl, cycloalkyl, aryl, aralkyl, heteroaryl, heterocyclyl, heteroarylalkyl or heterocyclylalkyl;
R₂ is oxygen, sulphur —NH, —N-acyl, —N(CN), —N(NO₂), —C(R₃)₂ or —CH(NO₂); [symbol]
represents a single bond or a double bond;
R₃ is hydrogen, alkyl, cycloalkyl, aryl, heterocyclyl, heteroaryl, aralkyl, heteroarylalkyl or heterocyclylalkyl;
R₄ cycloalkyl or heterocyclyl; and
Het is heterocyclyl.

4. The method of claim 3 further comprising administering one or more pharmaceutically acceptable carriers, excipients or diluents.

5. A method of claim 3, wherein the autoimmune disease, inflammation or associated pathology is selected from sepsis, rheumatoid arthritis, inflammatory bowel disease, type-1 diabetes, asthma, chronic obstructive pulmonary disorder, organ transplant rejection or psoriasis.

6-18. (canceled)
